(19) United States
(12) Patent Application Publication
(10) Pub. No.: US 2009/0175906 A1
(43) Pub. Date: Jul. 9, 2009

(54) Genetically Modified Attenuated Vesicular Stomatitis Virus, Compositions and Methods of use Thereof

(75) Inventors: Narender Kumar Kalyan, Ridgewood, NJ (US); Irina Yurgelonis, Ridgewood, NJ (US); Roger Michael Hendry, Atlanta, GA (US); Mark William Cutler, Bloomingdale, NJ (US); Kristen Elissa Syvertsen, Tarrytown, NY (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/338,367

(22) Filed: Dec. 18, 2008

Related U.S. Application Data
(60) Provisional application No. 61/015,868, filed on Dec. 21, 2007.

(51) Int. Cl. A61K 39/12; C12N 7/01; C12N 7/08

(52) U.S. Cl. 424/205.1; 435/235.1; 435/237

(57) ABSTRACT

The present invention relates to methods for generating genetically modified and attenuated strains of vesicular stomatitis virus (VSV) for use in the preparation of immunogenic compositions. More particularly, the invention relates to the identification of particular genetic modifications of attenuated VSV that result in an increased yield of virus and an increase in stability of the attenuated strains for preparation of the immunogenic compositions. Methods for cell culture propagation and use in large scale production of VSV is also disclosed.

### CROSS-REFERENCE TO RELATED APPLICATIONS

This application claims priority to U.S. provisional application No. 61/015,868, filed on Dec. 21, 2007, which is incorporated herein by reference in its entirety.

### GOVERNMENT SUPPORT CLAUSE

The research leading to the present invention was supported, at least in part, by National Institutes of Health contract number N01-A1-25458. Accordingly, the Government may have certain rights in the invention.

### FIELD OF THE INVENTION

The present invention relates generally to negative-strand RNA viruses. In particular, the invention relates to methods and compositions for adapting Vesicular Stomatitis Virus (VSV) particles to growth in cell culture for use in production.

### BACKGROUND TO THE INVENTION

Vesicular stomatitis virus (VSV) is a prototypic virus of the Rhabdoviridae family, belonging to the order Mononegavirales, which includes single stranded, non-segmented, negative-sense RNA viruses with highly conserved gene order. The 11-kb VSV genome contains five genes encoding five viral proteins: the nucleocapsid protein (N), the phosphoprotein (P), the matrix protein (M), the attachment glycoprotein (G), and the RNA-dependent RNA polymerase (L). The gene order in the genome is 3′-N—P-M-G-L-5′ and a number of studies have demonstrated that gene expression is obligatorily sequential from a single 3′ promoter (Rose and Whitt, Rhabdoviridae: The Viruses and Their Replication. In “Fields Virology”, 4^{th} Edition Vol. 1. Lippincott and Williams and Wilkins, 1221-1244, 2001).

The N gene encodes the nucleocapsid protein responsible for encapsidating the genome while the P (phosphoprotein) and L (large) coding sequences specify subunits of the RNA-dependent RNA polymerase. The matrix protein (M) promotes virion maturation and lines the inner surface of the virus particle. VSV encodes a single envelope glycoprotein (G), which serves as the cell attachment protein, mediates membrane fusion, and is the target of neutralizing antibodies.

The two most common serotypes of VSV in the western hemisphere are designated as Indiana (VSVin) and New Jersey (VSVnj). In nature, VSV infects livestock causing a self-limiting disease. Although naturally occurring human infections with VSV are infrequent, cases of VSV infections have been reported for individuals directly exposed to infected livestock or within laboratory environments. VSV infection of humans is typically asymptomatic or results in a mild influenza-like illness (Fields, B. N., and K. Hawkins, N. Engl. J. Med. 277:989-94, 1967). Among small mammals, mice can easily be infected experimentally via a variety of inoculation routes and thus serve as an excellent small animal model for immunogenicity, pathogenicity and neurovirulence studies (Bruno-Lobo, et al. An. Microbiol. (Rio J.) 15:53-68, 1968; Bruno-Lobo, et al., An. Microbiol. (Rio J.) 15:69-80, 1968; Flanagan, E. B. et al. J. Virol. 77:5740-8, 2003; Wagner, R. R. Infect. Immun. 10:309-315, 1974; Huneycutt, et al. J. Virol. 67:6698-706, 1993).

In the past few years, VSV has demonstrated promise as a vector for immunogenic compositions containing a number of human pathogens including HIV, papilloma virus, RSV, hepatitis C virus and influenza virus. Numerous properties make VSV an attractive candidate vector for human use (Bukreyev, et al. J. Virol. 80:10293-306, 2006; Clarke, et al. Springer Semin Immunopathol. 28: 239-253, 2006). These properties include: 1) VSV is not a human pathogen; 2) there is little pre-existing immunity that might impede its use in humans; 3) VSV readily infects many cell types; 4) it propagates efficiently in cell lines suitable for manufacturing immunogenic compositions; 5) it is genetically stable; 6) methods exist by which recombinant virus can be produced; 7) VSV can accept one or more foreign gene inserts and direct high levels of expression upon infection; and 8) VSV infection is an efficient inducer of both cellular and humoral immunity. Such studies have been greatly facilitated by the advent of a reverse genetics technique that allows for easy recovery of rVSV from genomic cDNA (Lawson, et al. Proc Natl Acad Sci USA 92:4477-81, 1995; Schnell, et al. EMBO J. 13:4195-203, 1994). In addition, the relatively small and simple genome organization of VSV has proven amenable to foreign gene insertion, with the resulting viruses producing high levels of foreign protein. The first vectors were designed with foreign coding sequence inserted between the G and L genes along with the requisite intergenic transcriptional control elements. These prototype vectors were found to elicit potent immune responses against the foreign antigen and were well tolerated in the animal models in which they were tested (Grigera, et al. Virus Res 69:3-15, 2000; Kahn et al. J Virol 75:11079-87, 2001; Roberts, et al. J Virol 73:3723-32, 1999; Roberts, et al. J Virol 72:4704-11, 1998, Rose, et al. Cell 106:539-49, 2001; Rose, et al. J Virol 74:10903-10, 2000; Schlereth, et al. J Virol 74:4652-7, 2000). Notably, Rose et al. found that coadministration of two vectors, one encoding HIV-1 env and the other encoding SIV gag, produced immune responses in immunized macaques that protected against challenge with a pathogenic SHIV (Rose, et al. Cell 106:539-49, 2001). Most of these studies were conducted with prototypic VSV vectors that were derived from a wild type (wt) VSV backbone and were shown to be significantly attenuated compared to the wt VSV (Roberts, et al. J. Virol. 72: 4704-11, 1998). More recent studies showed that prototype VSV vectors, when evaluated in a non-human primate model for neurovirulence, caused a significant level of injury to neurological tissues, albeit at reduced levels compared to the wild type virus (Johnson, et al. Virol. 360, 36-49, 2007). These observations led to the conclusion that the prototype rVSV vector might not be adequately attenuated for use in humans.

The development of scaleable propagation methods that are compliant with regulations governing manufacture of immunogenic compositions for administration to humans remains a hurdle that must be addressed before clinical evaluation can be justified. When designing a vector for administration to humans, it is imperative that the mutations that result in virus attenuation are stable and that the yields of virus are sufficient for scaled-up production. A single human dose is expected to be at least 1×10⁷ IUs, thus, manufacturing of a vector will be practical only if greater than 10⁷ IUs are produced per ml of culture medium.

There is a need in the art for methods of adapting attenuated VSV particles for increased growth in cell culture, wherein the yields of attenuated VSV particles recovered are sufficient to be of use in large-scale manufacture. Desirably, such methods would employ cells qualified for commercial production. In addition, such methods should retain the original mutations that resulted in virus attenuation, while at the same time improving the yields to sufficient levels for scaled-up production.

The citation of any reference herein should not be deemed as an admission that such reference is available as prior art to the instant invention.

### SUMMARY OF THE INVENTION

In accordance with the present invention, there is provided a process or a method for adaptation of a highly attenuated VSV recombinant to tissue culture conditions by continuous passaging at low multiplicity of infection (MOI) in Vero cells or in any susceptible cell substrate. The multiple serial passaging process results in genotypic changes characterized by progressive accrual of a number of nucleotide (NT) substitutions throughout the viral genome. Most of these nucleotide substitutions result in amino acid (AA) substitutions in the VSV proteins. This process resulted in phenotypic adaptation of the virus, accompanied by substantial improvements in virus yield. Passage in Vero cells is continued until genotypic and phenotypic stability is achieved, usually in 10 to 15 serial passages (P10-P15). Further passaging of the virus beyond P15 showed few or no additional substitutions and did not result in further enhancement of virus yields. This process results in substantial improvement in manufacturing yield as well as enhanced manufacturing consistency. The adaptive mutations did not substantially affect the neurovirulence (NV) of the passaged virus when tested in the highly sensitive mouse intracranial NV animal model

Accordingly, one aspect of the invention provides an isolated, genetically modified vesicular stomatitis virus (VSV) having at least one amino acid mutation in a region corresponding to at least one of the following positions:

the amino acids at positions 119 or 142 of the M protein;

the amino acids at positions 109, 224, 438, 477, or 481 of the G protein; and

the amino acids at positions 205, 220 or 1450 of the L protein.

In one embodiment of the invention, the nucleic acid encoding the genetically modified VSV further comprises a nucleic acid encoding at least one heterologous antigen, or a fragment thereof. It is envisioned that the one heterologous antigen, or a fragment thereof, is from a pathogenic microorganism. The pathogenic microorganism from which the nucleic acid encoding the heterologous antigen is obtained may be selected from the group consisting of a virus, a bacterium, a protozoan and a fungus. In one embodiment, the heterologous antigen may be selected from the group consisting of a human immunodeficiency virus (HIV) antigen, an HTLV antigen, an SIV antigen, an RSV antigen, a PIV antigen, an HSV antigen, a CMV antigen, an Epstein-Barr virus antigen, a Varicella-Zoster virus antigen, a mumps virus antigen, a measles virus antigen, an influenza virus antigen, a poliovirus antigen, a rhinovirus antigen, a hepatitis A virus antigen, a hepatitis B virus antigen, a hepatitis C virus antigen, a Norwalk virus antigen, a togavirus antigen, an alphavirus antigen, a rubella virus antigen, a rabies virus antigen, a Marburg virus antigen, an Ebola virus antigen, a papilloma virus antigen, a polyoma virus antigen, a metapneumovirus antigen, a coronavirus antigen, a Vibrio cholerae antigen, a Plasmodium falciparum antigen, a Plasmodium vivax antigen, a Plasmodium ovale antigen, a Plasmodium malariae antigen, a Plasmodium knowlesi antigen, a Streptococcus pneumoniae antigen, Streptococcus pyogenes antigen, a Helicobacter pylori antigen, a Streptococcus agalactiae antigen, a Neisseria meningitidis antigen, a Neisseria gonorrhoeae antigen, a Corynebacterium diphtheriae antigen, a Clostridium tetani antigen, a Bordetella pertussis antigen, a Haemophilus antigen, a Chlamydia antigen and an Escherichia coli antigen.

In one embodiment of the invention, the nucleic acid encoding the genetically modified VSV further comprises a nucleic acid encoding a heterologous antigen that is a human immunodeficiency virus (HIV) protein. In one embodiment, the HIV protein is encoded by a gene selected from the group consisting of gag, env, pol, vif, nef, tat, vpr, rev and vpu. In one embodiment, the HIV protein is an HIV gag protein.

In one embodiment, the HIV gag protein has at least one mutation at position 165, 270, 329, or 348.

In one embodiment, the genetically modified VSV has a mutation that comprises a conservative or non-conservative amino acid change.

In one embodiment, the genetically modified VSV has a mutation that is at either position 119 or 142 of the M protein or is at both positions 119 and 142 of the M protein. In one embodiment, the mutation of the amino acid at position 119 of the M protein is a T→N mutation and the mutation of the amino acid at position 142 of the M protein is a P→T mutation or a P→Q mutation.

In one embodiment, the genetically modified VSV has a mutation of the amino acids at position 109, 224, 438, 477 or 481 of the G protein that is a K N, N→T, S→I, A→V/G→L or V→I mutation, respectively.

In one embodiment, the genetically modified VSV has a mutation of the amino acid at position 205, 220 or 1450 of the L protein that is P→L, K→E, or L→I, respectively.

In one embodiment, the genetically modified VSV further comprises a nucleic acid molecule encoding the HIV gag protein, wherein the HIV gag protein has a mutation in at least one of the amino acids at position 165, 270, 329 or 348, wherein the mutation is S→G, L→S, D→N or T→K, respectively.

In one embodiment, the mutations noted above in the genetically modified VSV result in increased stability of the virus genotype and/or phenotype. In one embodiment, the mutations noted above in the genetically modified VSV further result in increased yield in virus production from a cell infected with the genetically modified VSV.

In one embodiment, the genetically modified VSV further comprises at least two other mutations in its genome. In one embodiment, the mutations may be selected from the group consisting of a temperature-sensitive mutation, a point mutation, a gene shuffling mutation, a G-stem mutation, a non-cytopathic M gene mutation, an ambisense RNA mutation, a truncated G gene mutation, a G gene insertion mutation and a gene deletion mutation.

A second aspect of the invention provides a method for producing the genetically modified VSV as described herein, the method comprising serial passaging of a VSV in a susceptible mammalian cell line at a low multiplicity of infection (MOI) ranging from about 0.001 to about 0.1 plaque forming units (PFU)/ml for at least 5-15 passages, wherein the virus has a titer of at least 1×10⁶ PFU/ml and at least one or more of the mutations as described herein.

In one embodiment, the method described above results in a genetically modified and attenuated virus that has a titer of at least 1×10⁷ PFU/ml.

In one embodiment, the method described above utilizes a susceptible cell line, that is, any cell line that is capable of being infected with the genetically modified VSV as described herein. For example, a susceptible cell line may include, but is not limited to, a Vero cell line, baby hamster kidney (BHK) cells, or a human embryonic kidney cell line, such as, a 293 cell line.

In one embodiment, the method described above results in a 5 to 100 fold higher yield of virus compared to that obtained with a virus strain that has not been passaged for about 5 to 15 times at a low MOI ranging from about 0.001 to about 0.1 plaque forming units (PFU)/ml.

In one embodiment, the method described above results in an increase in stability of the virus genotype and/or phenotype.

A third aspect of the invention provides an immunogenic composition comprising any one or more of the genetically modified VSV described above and a pharmaceutically acceptable carrier.

In one embodiment, the immunogenic composition further comprises an adjuvant.

A fourth aspect of the invention provides a method for protecting a mammal against infection with a pathogenic microorganism, the method comprising administering an immunologically effective amount of any one or more of the genetically modified VSV as described herein.

A fifth aspect of the invention provides a method for adapting a virus for growth in cell culture comprising
a. infecting the cell culture with the virus at a low multiplicity-of-infection (MOI) ranging from about 0.001 to about 0.1 plaque forming units (PFU) per cell;
b. harvesting the cell culture medium containing the virus;
c. clarifying the cell culture medium;
d. freezing the cell culture medium; and
e. repeating steps a) through d) for about 5 to about 15 times,
wherein the method results in a 5 to 100 fold increase in virus production/yield and an increase in the stability of the virus genotype and phenotype.

In one embodiment, the method described herein utilizes a virus that is an attenuated virus. In one embodiment, the method is adapted for large scale production of a viral immunogenic composition. In one embodiment, the method results in a 5 to 100 fold higher yield of virus compared to that obtained with a virus strain that has not been passaged for about 5 to 15 times at a low multiplicity of infection ranging from about 0.001 to about 0.1 plaque forming units per cell. In one embodiment, the method described above allows for maintaining any pre-existing mutation(s) associated with virus attenuation. The pre-existing mutation(s) associated with virus attenuation may be selected from the group consisting of a temperature-sensitive mutation, a point mutation, a gene shuffling mutation, a G-stem mutation, a non-cytopathic M gene mutation, an ambisense RNA mutation, a truncated G gene mutation, a G gene insertion mutation and a gene deletion mutation. In one embodiment, the method allows for maintaining a low neurovirulence profile associated with virus attenuation. In one embodiment, the attenuated virus used in the methods described above is a strain of vesicular stomatitis virus (VSV). In one embodiment, the methods described above utilize a genetically modified VSV that has at least one amino acid mutation in a region corresponding to at least one of the following positions:
the amino acids at positions 119 or 142 of the M protein;
the amino acids at positions 109, 224, 438, 477, or 481 of the G protein; and
the amino acids at positions 205, 220 or 1450 of the L protein.

In one embodiment, the method described herein utilizes a Vesicular Stomatitis Virus that has a mutation that comprises a conservative or non-conservative amino acid change. In one embodiment, the mutation may be at either position 119 or 142 of the VSV M protein or is at both positions 119 and 142 of the M protein. In one embodiment, the mutation of the amino acid at position 119 of the M protein is a T→N mutation and the mutation of the amino acid at position 142 of the M protein is a P→T or a P→Q mutation.

In one embodiment, the mutation may be at position 109, 224, 438, 477 or 481 of the VSV G protein and may be a K→N, N→T, S→I, A→V/G→L, or V→I mutation, respectively.

In one embodiment, the mutation may be at position 205, 220 or 1450 of the VSV L p

In one embodiment of the invention, the methods described herein utilize a strain of VSV that may be selected from an Indiana serotype (ATCC, VR-1238), a New Jersey serotype (ATCC, VR-1239), an Isfahan serotype (PMID: 192094), a Chandipura serotype (ATCC, VR-476) or other vesiculoviruses.

In one embodiment of the invention, the methods described herein utilize a strain of VSV that contains a nucleic acid encoding at least one heterologous antigen. The heterologous antigen is obtained from a pathogenic microorganism selected from the group consisting of a virus, a bacterium, a protozoan and a fungus. The heterologous antigen may be selected from the group consisting of a human immunodeficiency virus (HIV) antigen, an HTLV antigen, an SIV antigen, an RSV antigen, a PIV antigen, an HSV antigen, a CMV antigen, an Epstein-Barr virus antigen, a Varicella-Zoster virus antigen, a mumps virus antigen, a measles virus antigen, an influenza virus antigen, a poliovirus antigen, a rhinovirus antigen, a hepatitis A virus antigen, a hepatitis B virus antigen, a hepatitis C virus antigen, a Norwalk virus antigen, a togavirus antigen, an alphavirus antigen, a rubella virus antigen, a rabies virus antigen, a Marburg virus antigen, an Ebola virus antigen, a papilloma virus antigen, a polyoma virus antigen, a metapneumovirus antigen, a coronavirus antigen, a Vibrio cholerae antigen, a Plasmodium falciparum antigen, a Plasmodium vivax antigen, a Plasmodium ovale antigen, a Plasmodium malariae antigen, a Plasmodium knowlesi antigen, a Streptococcus pneumoniae antigen, Streptococcus pyogenes antigen, a Helicobacter pylori antigen, a Streptococcus agalactiae antigen, a Neisseria meningitidis antigen, a Neisseria gonorrhoeae antigen, a Corynebacterium diphtheriae antigen, a Clostridium tetani antigen, a Bordetella pertussis antigen, a Haemophilus antigen, a Chlamydia antigen, and an Escherichia coli antigen.

In one embodiment, the heterologous antigen comprises an HIV protein. In one embodiment, the HIV protein is encoded by a gene selected from the group consisting of gag, env, pol, vif, nef, tat, vpr, rev and vpu. In one embodiment, the HIV protein is an HIV gag protein. In one embodiment, the HIV gag protein has at least one mutation at position 165, 270, 329 or 348.

In one embodiment, the mutation of the amino acid at position 165, 270, 329 or 348 of the HIV gag protein is S→G, L→S, D→N, or T→K, respectively.

### BRIEF DESCRIPTION OF THE DRAWINGS

FIG. 1: Schematic genomic organization of wt VSV and attn VSVN4CT1-gag1 (IN and NJ serotypes).

FIG. 2: Outline of the experimental protocol used to serially passage virus in Vero cells. The viruses at every 5^{th} passage were analyzed by indicated assays.

FIG. 3: Adaptive amino acid substitutions accrued in IN serotype of attenuated VSV (rVSVinN4CT1-gag1) virus following serial passage in Vero cells.

FIG. 4: Adaptive amino acid substitutions accrued in NJ serotype of attenuated VSV (rVSVnjN4CT1-gag1) virus following serial passage in Vero cells.

FIG. 5: Effect of passage levels on growth kinetics of IN serotype of attenuated rVSVinN4CT1-gag1 virus. Serial passage resulted in a significant increase in yields that exceeded the manufacturing target of ≧10⁷ PFU/ml. There was no significant change in growth after passage 15.

FIG. 6: Effect of passage levels on growth kinetics of NJ serotype of attenuated rVSVnjN4CT1-gag1 virus.

FIG. 7: Results of neurovirulence (NV) testing of rVSVinN4CT1-gag1 passaged viruses P0 to P25 in the mouse intracranial (IC) LD₅₀ animal model as described in Cooper et al., J Virology, 82, 207-29, 2008.

FIG. 8A-8L: Comparison of the nucleotide (NT) and amino acid (AA) sequences of original (passage 0 or P0) viruses and passage 25 of VSV Indiana serotype. The NT and AA substitutions in the passaged virus are shown in bold.

FIG. 9A-9M: Comparison of the nucleotide (NT) and amino acid (M) sequences of original (passage 0 or P0) viruses and passage 25 of VSV New Jersey serotype. The NT and AA substitutions in the passaged virus are shown in bold.

FIG. 10: Growth Kinetics of VSVinN4CT1-gag1 Bioreactor Runs, Using Low and High Passage Virus

FIG. 11: Growth Kinetics of VSVnjN4CT1-gag1 Bioreactor Runs, Using Low and High Passage Virus

### DETAILED DESCRIPTION OF THE INVENTION

Before the present methods and treatment methodology are described, it is to be understood that this invention is not limited to particular methods, and experimental conditions described, as such methods and conditions may vary. It is also to be understood that the terminology used herein is for purposes of describing particular embodiments only, and is not intended to be limiting.

As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth.

Accordingly, in the present application, there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (1994).

Although any methods and materials similar or equivalent to those described herein can be used in the practice or testing of the invention, the preferred methods and materials are now described. All publications mentioned herein are incorporated by reference in their entirety.

### DEFINITION

The terms used herein have the meanings recognized and known to those of skill in the art, however, for convenience and completeness, particular terms and their meanings are set forth below.

The term “about” means within 20%, preferably within 10%, and more preferably within 5%.

The term “adjuvant” refers to a compound or mixture that enhances the immune response to an antigen. An adjuvant can serve as a tissue depot that slowly releases the antigen and also as a lymphoid system activator that non-specifically enhances the immune response (Hood et al., Immunology, Second Ed., 1984, Benjamin/Cummings: Menlo Park, Calif., p. 384). Depending on the circumstances, a primary challenge with an antigen alone, in the absence of an adjuvant, may fail to elicit a sufficient humoral or cellular immune response. A number of cytokines or lymphokines have been shown to have immune modulating activity, and thus are useful as adjuvants, including, but not limited to, the interleukins 1-α, 1-β, 2, 4, 5, 6, 7, 8, 10, 12 (see, e.g., U.S. Pat. No. 5,723,127), 13, 14, 15, 16, 17 and 18 (and its mutant forms); the interferons-α, β and γ; granulocyte-macrophage colony stimulating factor (GM-CSF) (see, e.g., U.S. Pat. No. 5,078,996 and ATCC Accession Number 39900); macrophage colony stimulating factor (M-CSF); granulocyte colony stimulating factor (G-CSF); and the tumor necrosis factors α and β. Still other adjuvants that are useful with the immunogenic compositions described herein include chemokines, including without limitation, MCP-1, MIP-1α, MIP-1β, and RANTES; adhesion molecules, such as a selectin, e.g., L-selectin, P-selectin and E-selectin; mucin-like molecules, e.g., CD34, GlyCAM-1 and MadCAM-1; a member of the integrin family such as LFA-1, VLA-1, Mac-1 and p150.95; a member of the immunoglobulin superfamily such as PECAM, ICAMs, e.g., ICAM-1, ICAM-2 and ICAM-3, CD2 and LFA-3; co-stimulatory molecules such as CD40 and CD40L; growth factors including vascular growth factor, nerve growth factor, fibroblast growth factor, epidermal growth factor, B7.2, PDGF, BL-1, and vascular endothelial growth factor; receptor molecules including Fas, TNF receptor, Flt, Apo-1, p55, WSL-1, DR3, TRAMP, Apo-3, AIR, LARD, NGRF, DR4, DR5, KILLER, TRAIL-R2, TRICK2, and DR6; and Caspase (ICE).

Suitable adjuvants used to enhance an immune response further include, without limitation, MPL™ (3-O-deacylated monophosphoryl lipid A, Corixa, Hamilton, Mont.), which is described in U.S. Pat. No. 4,912,094. Also suitable for use as adjuvants are synthetic lipid A analogs or aminoalkyl glucosamine phosphate compounds (AGP), or derivatives or analogs thereof, which are available from Corixa (Hamilton, Mont.), and which are described in U.S. Pat. No. 6,113,918. One such AGP is 2-[(R)-3-Tetradecanoyloxytetradecanoylamino]ethyl 2-Deoxy-4-O-phosphono-3-O—[(R)-3-tetradecanoyoxytetradecanoyl]-2-[(R)-3-tetradecanoyloxytetradecanoyl-amino]-b-D-glucopyranoside, which is also known as 529 (formerly known as RC529). This 529 adjuvant is formulated as an aqueous form (AF) or as a stable emulsion (SE).

Still other adjuvants include muramyl peptides, such as N-acetyl-muramyl-L-threonyl-D-isoglutamine (thr-MDP), N-acetyl-normuramyl-L-alanine-2-(1′-2′ dipalmitoyl-sn-glycero-3-hydroxyphosphoryloxy)-ethylamine (MTP-PE); oil-in-water emulsions, such as MF59 (International PCT Publication No. WO 90/14837) (containing 5% Squalene, 0.5% Tween 80, and 0.5% Span 85 (optionally containing various amounts of MTP-PE) formulated into submicron particles using a microfluidizer such as Model 110Y microfluidizer (Microfluidics, Newton, Mass.)), and SAF (containing 10% Squalene, 0.4% Tween 80, 5% pluronic-blocked polymer L121, and thr-MDP, either microfluidized into a submicron emulsion or vortexed to generate a larger particle size emulsion); incomplete Freund's adjuvant (IFA); aluminum salts (alum), such as aluminum hydroxide, aluminum phosphate, aluminum sulfate; Amphigen; Avridine; L121/squalene; D-lactide-polylactide/glycoside; pluronic polyols; killed Bordetella; saponins, such as Stimulon™ QS-21 (Antigenics, Framingham, Mass.), described in U.S. Pat. No. 5,057,540, ISCOMATRIX (CSL Limited, Parkville, Australia), described in U.S. Pat. No. 5,254,339, and immunostimulating complexes (ISCOMS); Mycobacterium tuberculosis; bacterial lipopolysaccharides; synthetic polynucleotides such as oligonucleotides containing a CpG motif (e.g., U.S. Pat. No. 6,207,646); IC-31 (Intercell AG, Vienna, Austria), described in European Patent Nos. 1,296,713 and 1,326,634; a pertussis toxin (PT) or mutant thereof, a cholera toxin or mutant thereof (e.g., International PCT Publication Nos. WO00/18434, WO02/098368 and WO02/098369); or an E. coli heat-labile toxin (LT), particularly LT-K63, LT-R72, PT-K9/G129; see, e.g., International PCT Publication Nos. WO 93/13302 and WO 92/19265.

The term “antigen” refers to a compound, composition, or immunogenic substance that can stimulate the production of antibodies or a T-cell response, or both, in an animal, including compositions that are injected or absorbed into an animal. The immune response may be generated to the whole molecule, or to a portion of the molecule (e.g., an epitope or hapten). The term may be used to refer to an individual macromolecule or to a homogeneous or heterogeneous population of antigenic macromolecules. An antigen reacts with the products of specific humoral and/or cellular immunity. The term “antigen” broadly encompasses moieties including proteins, polypeptides, antigenic protein fragments, nucleic acids, oligosaccharides, polysaccharides, organic or inorganic chemicals or compositions, and the like. The term “antigen” includes all related antigenic epitopes. Epitopes of a given antigen can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715, all incorporated herein by reference in their entireties. Similarly, conformational epitopes are readily identified by determining spatial conformation of amino acids such as by, e.g., x-ray crystallography and 2-dimensional nuclear magnetic resonance. See, e.g., Epitope Mapping Protocols, supra. Furthermore, for purposes of the present invention, an “antigen” can also be used to refer to a protein that includes modifications, such as deletions, additions and substitutions (generally conservative in nature, but they may be non-conservative), to the native sequence, so long as the protein maintains the ability to elicit an immunological response. These modifications may be deliberate, as through site-directed mutagenesis, or through particular synthetic procedures, or through a genetic engineering approach, or may be accidental, such as through mutations of hosts, which produce the antigens. Furthermore, the antigen can be derived or obtained from any virus, bacterium, parasite, protozoan, or fungus, and can be a whole organism. Similarly, an oligonucleotide or polynucleotide, which expresses an antigen, such as in nucleic acid immunization applications, is also included in the definition. Synthetic antigens are also included, for example, polyepitopes, flanking epitopes, and other recombinant or synthetically derived antigens (Bergmann et al. (1993) Eur. J. Immunol. 23:2777 2781; Bergmann et al. (1996) J. Immunol. 157:3242 3249; Suhrbier, A. (1997) Immunol. and Cell Biol. 75:402 408; Gardner et al. (1998) 12th World AIDS Conference, Geneva, Switzerland, Jun. 28 Jul. 3, 1998).

The term “attenuated” refers to a strain of pathogen whose pathogenicity has been reduced so that it will initiate an immune response without producing the specific disease. An attenuated strain of a virus is less virulent than the parental strain from which it is derived. Conventional means are used to introduce attenuating mutations to generate a modified virus, such as chemical mutagenesis during virus growth in cell cultures to which a chemical mutagen has been added. An alternative means of introducing attenuating mutations comprises making pre-determined mutations using site-directed mutagenesis. One or more mutations may be introduced. These viruses are then screened for attenuation of their biological activity in cell culture and/or in an animal model. If the attenuated phenotype of the rescued virus is present, challenge experiments can be conducted with an appropriate animal model. Non-human primates can serve as an appropriate animal model for the pathogenesis of human disease. These primates are first immunized with the attenuated, recombinantly-produced virus, then challenged with the wild-type form of the virus.

The terms “cell”, “host cell”, “cell culture” and the like as used herein are intended to include any individual cell or cell culture which can be or have been recipients for viruses, vectors or the incorporation of exogenous nucleic acid molecules, polynucleotides and/or proteins. It is also intended to include progeny of a single cell. However, the progeny may not necessarily be completely identical (in morphology or in genomic or total DNA complement) to the original parent cell due to natural, accidental, or deliberate mutation. The cells are preferably eukaryotic, but may be prokaryotic and include, but are not limited to, bacterial cells, yeast cells, animal cells, and mammalian cells (e.g., murine, rat, simian or human).

The term “clarifying”, as used herein, refers to an early step in virus purification, whereby the cells and cellular debris is removed after infection of cells or a cell culture with the virus of the present invention. For example, an early step in virus purification involves “clarifying” the cell culture medium to remove cell debris using a method such as low speed centrifugation (≦10,000 RPM) or filtration. The virus present in the supernatant is then isolated and purified using methods known to those skilled in the art, such as high speed centrifugation (eg. 100,000×g) through a sucrose cushion or isolation through an ion exchange column, such as that described in U.S. Patent Publication 20070249019.

It is noted that in this disclosure, terms such as “comprises”, “comprised”, “comprising”, “contains”, “containing” and the like can have the meaning attributed to them in U.S. patent law; eg., they can mean “includes”, “included”, “including” and the like. Terms such as “consisting essentially of” and “consists essentially of” have the meaning attributed to them in U.S. patent law, eg., they allow for the inclusion of additional ingredients or steps that do not detract from the novel or basic characteristics of the invention, ie., they exclude additional unrecited ingredients or steps that detract from novel or basic characteristics of the invention, and they exclude ingredients or steps of the prior art, such as documents in the art that are cited herein or are incorporated by reference herein, especially as it is a goal of this document to define embodiments that are patentable, eg., novel, nonobvious, inventive, over the prior art, eg., over documents cited herein or incorporated by reference herein. And, the terms “consists of” and “consisting of” have the meaning ascribed to them in U.S. patent law; namely, that these terms are closed ended.

A “conservative amino acid substitution” refers to the substitution of one or more of the amino acid residues of a protein with other amino acid residues having similar physical and/or chemical properties. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. Amino acids containing aromatic ring structures are phenylalanine, tryptophan, and tyrosine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such alterations will not be expected to affect apparent molecular weight as determined by polyacrylamide gel electrophoresis, or isoelectric point. Particularly preferred substitutions are: Lys for Arg and vice versa such that a positive charge may be maintained; Glu for Asp and vice versa such that a negative charge may be maintained; Ser for Thr such that a free—OH can be maintained; and Gln for Asn such that a free NH₂ can be maintained.

The terms “culture fluid”, “cell culture fluid”, “cell culture media”, “media” and/or “bioreactor fluid” are used interchangeably, and refer to the media or solution in which the cell culture is grown.

“Encoded by” or “encoding” refers to a nucleic acid sequence which codes for a polypeptide sequence, wherein the polypeptide sequence contains an amino acid sequence of at least 3 to 5 amino acids, more preferably at least 8 to 10 amino acids, and even more preferably at least 15 to 20 amino acids, a polypeptide encoded by the nucleic acid sequences. Also encompassed are polypeptide sequences, which are immunologically identifiable with a polypeptide encoded by the sequence. Thus, an antigen “polypeptide,” “protein,” or “amino acid” sequence may have at least 70% similarity, preferably at least about 80% similarity, more preferably about 90-95% similarity, and most preferably about 99% similarity, to a polypeptide or amino acid sequence of an antigen.

“Fragment” refers to either a protein or polypeptide comprising an amino acid sequence of at least 4 amino acid residues (preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues) of the amino acid sequence of a parent protein or polypeptide, or a nucleic acid comprising a nucleotide sequence of at least 10 base pairs (preferably at least 20 base pairs, at least 30 base pairs, at least 40 base pairs, at least 50 base pairs, at least 50 base pairs, at least 100 base pairs, at least 200 base pairs) of the nucleotide sequence of the parent nucleic acid. Any given fragment may or may not possess a functional activity of the parent nucleic acid or protein or polypeptide.

A “gene” as used in the context of the present invention is a sequence of nucleotides in a nucleic acid molecule (chromosome, plasmid, etc.) with which a genetic function is associated. A gene is a hereditary unit, for example of an organism, comprising a polynucleotide sequence (e.g., a DNA sequence for mammals) that occupies a specific physical location (a “gene locus” or “genetic locus”) within the genome of an organism. A gene can encode an expressed product, such as a polypeptide or a polynucleotide (e.g., tRNA). Alternatively, a gene may define a genomic location for a particular event/function, such as the binding of proteins and/or nucleic acids (e.g., phage attachment sites), wherein the gene does not encode an expressed product. Typically, a gene includes coding sequences, such as polypeptide encoding sequences, and non-coding sequences, such as promoter sequences, poly-adenylation sequences, transcriptional regulatory sequences (e.g., enhancer sequences). Many eucaryotic genes have “exons” (coding sequences) interrupted by “introns” (non-coding sequences). In certain cases, a gene may share sequences with another gene(s) (e.g., overlapping genes). The term “gene” may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription.

As defined herein, the terms “gene shuffling”, “shuffled gene”, “shuffled”, “shuffling”, “gene rearrangement” and “gene translocation” are used interchangeably, and refer to a change (mutation) in the order of the wild-type VSV genome. As defined herein, a wild-type VSV genome has the following gene order: 3′-NPMGL-5′.

The term “genetically modified” generally refers to the introduction of one or more mutations into the virus genome by any means known to those skilled in the art. However, while certain “genetic modifications” may be made by specific deletions, insertions, or substitutions, or by transfer of genetic material into the viral genome using standard genetic engineering techniques, certain genetic modifications of the VSV genome in the present invention occurred by continuous passage of an attenuated VSV at a low multiplicity of infection. This serial passage at low MOI resulted in the progressive accrual of a number of nucleotide substitutions throughout the viral genome and also resulted in amino acid substitutions in the VSV proteins. These “genetically modified” VSV particles were shown to be adapted for increased growth in cells, but without increased neuropathology in small animal models for neurovirulence. In the present invention, “low passage virus”, “passage 0” or “P0”, and “original virus” are used interchangeably and represent the rVSVN4CT1gag1 virus that served as the starting material for the genetically modified virus of the current invention. In some cases, particularly in the bioreactor runs, these also include viruses passaged 1 to 3 times to indicate low passage viruses. “High passage virus”, “passaged virus”, “genetically modified virus”, “tissue culture-adapted” or “cell-adapted virus” are used interchangeably and represent virus that has been passaged more than 5 times, generally 5 to 25 times, preferably 15 times.

The term “growing” or “growth” as used herein refers to the in vitro propagation of virus in cells of various kinds. The growing/growth of virus in cells in the laboratory involves inoculating the cells with the virus, followed by incubating to allow virus production and then harvesting the cell culture medium containing the virus. Virus-infected cells are normally grown in a growth medium within culture vessels (such as flasks or dishes for adherent cells or constantly moving bottles or flasks for cells in suspension) and the cultures are maintained in cell incubators with constant temperature, humidity and gas composition. However, culture conditions can vary depending on the cell type and can be altered to induce changes in the cells or to support or enhance virus production by the cells.

The term “harvesting”, as used herein, refers to the collection of cells or cell culture medium in preparation for isolation and purification of virus following the infection of a cell or cell line with any of the virus strains or serotypes described herein.

The term “heterologous” refers to a combination of elements not naturally occurring in a virus or cell. For example, heterologous DNA refers to the DNA not naturally located in the cell, or in a chromosomal site of the cell. The heterologous DNA may include a gene foreign to the cell. The term “heterologous antigen” as used herein is an antigen encoded in a nucleic acid sequence, wherein the antigen is either not from the organism, or is not encoded in its normal position or its native form. A heterologous expression regulatory element is an element operatively associated with a different gene than the one it is operatively associated within nature.

The term “immunogenic composition” relates to any pharmaceutical composition containing an antigen, eg. a microorganism, which composition can be used to elicit an immune response in a mammal. The immune response can include a T cell response, a B cell response, or both a T cell and B cell response. The composition may serve to sensitize the mammal by the presentation of antigen in association with MHC molecules at the cell surface. In addition, antigen-specific T-lymphocytes or antibodies can be generated to allow for the future protection of an immunized host. An “immunogenic composition” may contain a live, attenuated, or killed/inactivated formulation comprising a whole microorganism or an immunogenic portion derived therefrom that induces either a cell-mediated (T cell) immune response or an antibody-mediated (B cell) immune response, or both, and may protect the animal from one or more symptoms associated with infection by the microorganism, or may protect the animal from death due to the infection with the microorganism.

An “immunologically effective amount” or an “immunogenically effective amount” as used herein refers to the amount of antigen or formulation sufficient to elicit an immune response, either a cellular (T cell) or humoral (B cell or antibody) response, as measured by standard assays known to one skilled in the art. For example, with respect to the present invention, an “immunologically effective amount” is a minimal protection dose (titer) of ≧5.0 to 7.0 Log₁₀ pfu/mL. The effectiveness of an antigen as an immunogen, can be measured either by proliferation assays, by cytolytic assays, such as chromium release assays to measure the ability of a T cell to lyse its specific target cell, or by measuring the levels of B cell activity by measuring the levels of circulating antibodies specific for the antigen in serum. Furthermore, the level of protection of the immune response may be measured by challenging the immunized host with the antigen that has been injected. For example, if the antigen to which an immune response is desired is a virus or a tumor cell, the level of protection induced by the “immunologically effective amount” of the antigen is measured by detecting the percent survival or the percent mortality after viral, bacterial, protozoal, or fungal challenge of the animals.

The term “isolated” or “purified” means that the material is removed from its original environment (e.g., the natural environment if it is naturally occurring). For example, an “isolated” or “purified” peptide or protein is substantially free of cellular material or other contaminating proteins from the cell or tissue source from which the protein is derived, or substantially free of chemical precursors or other chemicals when chemically synthesized. In the present invention, the virus is isolated or purified from the infected cell or from cellular debris, so that it is provided in a form useful in the manufacture of an immunogenic composition. The language “substantially free of cellular material” includes preparations of a virus, or a polypeptide/protein in which the virus, or the polypeptide/protein is separated from cellular components of the cells from which it is isolated or recombinantly produced. Thus, a virus, or a polypeptide/protein that is substantially free of cellular material includes preparations of the virus or polypeptide/protein having less than about 30%, 20%, 10%, 5%, 2.5%, or 1%, (by dry weight) of contaminating protein. When the virus or polypeptide/protein is recombinantly produced, it is also preferably substantially free of culture medium, i.e., culture medium represents less than about 20%, 10%, 5%, 1%, 0.5%, or 0.2% of the volume of the protein preparation. When polypeptide/protein is produced by chemical synthesis, it is preferably substantially free of chemical precursors or other chemicals, i.e., it is separated from chemical precursors or other chemicals which are involved in the synthesis of the protein. Accordingly, such preparations of the polypeptide/protein have less than about 30%, 20%, 10%, 5% (by dry weight) of chemical precursors or compounds other than polypeptide/protein fragment of interest. An “isolated” or “purified” nucleic acid molecule is one which is separated from other nucleic acid molecules which are present in the natural source of the nucleic acid molecule. Moreover, an “isolated” nucleic acid molecule, such as a cDNA molecule or an RNA molecule, can be substantially free of other cellular material, or culture medium when produced by recombinant techniques, or substantially free of chemical precursors or other chemicals when chemically synthesized.

The term “multiplicity of infection” or “MOI” refers to the average number of viral particles that infects a single cell. The “MOI” is calculated by dividing the total number of viral plaque forming units (PFU) with the total number of cells being infected.

As defined hereinafter, the terms “mutation class”, “mutation classes” or “classes of mutation” are used interchangeably, and refer to mutations known in the art, when used singly, to attenuate VSV. For example, a “mutation class” of the invention includes, but is not limited to, a VSV temperature-sensitive N gene mutation (hereinafter, “N₍ₜₛ₎”), a temperature-sensitive L gene mutation (hereinafter, “L₍ₜₛ₎”), a point mutation, a G-stem mutation (hereinafter, “G₍ₛₜₑₘ₎”), a non-cytopathic M gene mutation (hereinafter, “M_{(ncp)}”), a gene shuffling or rearrangement mutation, a truncated G gene mutation (hereinafter, “G_{(ct)}”), an ambisense RNA mutation, a G gene insertion mutation, a gene deletion mutation and the like. As defined hereinafter, a “mutation” includes mutations known in the art as insertions, deletions, substitutions, gene rearrangement or shuffling modifications.

A “non-conservative amino acid substitution” refers to the substitution of one or more of the amino acid residues of a protein with other amino acid residues having dissimilar physical and/or chemical properties, using the characteristics defined above.

As used herein, the phrase “nucleic acid” or “nucleic acid molecule” refers to DNA, RNA, as well as any of the known base analogs of DNA and RNA or chimeras formed therefrom. Thus, a “nucleic acid” or a “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”) in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5N to 3N direction along the nontranscribed strand of DNA (i.e., the strand having a sequence homologous to the mRNA). A “recombinant DNA molecule” is a DNA molecule that has undergone a molecular biological manipulation.

The term “pathogenic” refers to the ability of any agent of infection, such as a bacterium or a virus, to cause disease. A “non-pathogenic” microorganism refers to a microorganism that lacks the disease causing characteristics for the “pathogenic” strains of a microorganism.

The term “pharmaceutically acceptable carrier” means a carrier approved by a regulatory agency of a Federal, a state government, or other regulatory agency, or listed in the U.S. Pharmacopeia or other generally recognized pharmacopeia for use in animals, including humans as well as non-human mammals. The term “carrier” refers to a diluent, adjuvant, excipient, or vehicle with which the pharmaceutical composition is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. The formulation should suit the mode of administration.

The term “plaque” or “viral plaque” refers to a clear, often round patch of lysed cells in an otherwise opaque layer of a cell culture. A “plaque-forming unit” or “PFU” refers to the average number of infectious viral particles per unit volume. For example, if a virus solution has 100 PFU/ml, this means that every one milliliter of this virus solution has 100 virus particles that can each form a plaque. PFU/ml is the conventional means to refer to a concentration of a plaque forming virus preparation. However, PFU is generally used interchangeably with “infectious unit” or “IU” and represents units of infectious virus in a virus preparation.

The term “protecting” refers to shielding eg. a mammal, from infection or a disease, by inducing an immune response to a particular pathogen. Such protection is generally achieved following treating a mammal with an immunogenic composition. The protection provided need not be absolute, i.e., the infection need not be totally prevented or eradicated, if there is a statistically significant improvement compared with a control population of mammals, e.g. infected animals not administered the immunogenic compositions. Protection may be achieved by mitigating the severity or rapidity of onset of symptoms of the infection.

The terms “protein”, “polypeptide” and “peptide” refer to a polymer of amino acid residues and are not limited to a minimum length of the product. Thus, peptides, oligopeptides, dimers, multimers, and the like, are included within the definition. Both full-length proteins and fragments thereof are encompassed by the definition. The terms also include modifications, such as deletions, additions and substitutions (generally conservative in nature, but which may be non-conservative), to a native sequence, preferably such that the protein maintains the ability to elicit an immunological response within an animal to which the protein is administered. Also included are post-expression modifications, eg. glycosylation, acetylation, phosphorylation and the like. The term “amino acid” refers to either natural and/or unnatural or synthetic amino acids, including both the D or L optical isomers, and amino acid analogs. The one letter and three letter codes for each of the natural amino acids are known to those skilled in the art.

Methods of producing recombinant RNA virus are referred to in the art as “rescue” or “reverse genetics” methods. Exemplary rescue methods for VSV are described in U.S. Pat. No. 6,033,886, U.S. Pat. No. 6,596,529 and WO 2004/113517, each incorporated herein by reference. The transcription and replication of negative-sense, single stranded, non-segmented, RNA viral genomes are achieved through the enzymatic activity of a multimeric protein complex acting on the ribonucleoprotein core (nucleocapsid). Naked genomic RNA cannot serve as a template. Instead, these genomic sequences are recognized only when they are entirely encapsidated by the N protein into the nucleocapsid structure. It is only in that context that the genomic and antigenomic terminal promoter sequences are recognized to initiate the transcriptional or replication pathways.

As defined hereinafter, the term “synergistic” attenuation refers to a level of VSV attenuation which is greater than additive. For example, a synergistic attenuation of VSV according to the present invention comprises combining at least two classes of mutation in the same VSV genome, thereby resulting in a reduction of VSV pathogenicity much greater than an additive attenuation level observed for each VSV mutation class alone. Thus, in certain embodiments, a synergistic attenuation of VSV is defined as a LD₅₀ at least greater than the additive attenuation level observed for each mutation class alone (i.e., the sum of the two mutation classes), wherein attenuation levels (i.e., the LD₅₀) are determined in a small animal neurovirulence model. Examples of synergistic attenuation of VSV are described in WO 2005/098009, incorporated herein by reference.

A VSV “temperature-sensitive” (“ts”) mutation, as defined hereinafter, is a mutation in the VSV genome, which restricts VSV growth at a non-permissive temperature. For example, a VSV ts mutant of the invention grows normally and to high titer at the permissive temperature (e.g., 31° C.), but their growth or reproduction is restricted at non-permissive temperatures (e.g., 37° C. or 39° C.).

The term “immunogenic composition” refers to pharmaceutical compositions that induce an immune response in an animal. An immunogenic composition may protect the animal from disease or possible death due to an infection, and may or may not include one or more additional components that enhance the immunological activity of the active component. An immunogenic composition may additionally comprise further components typical to immunogenic compositions, including, for example, an adjuvant or an immunomodulator. The immunogenically active component of an immunogenic composition may comprise complete live organisms in either their original form, or as attenuated organisms, or organisms inactivated by appropriate methods in a killed or inactivated immunogenic compositions, or subunit immunogenic compositions comprising one or more immunogenic components of the virus, or genetically engineered, mutated or cloned immunogenic compositions prepared by methods known to those skilled in the art. An immunogenic composition may comprise one or simultaneously more than one of the elements described above.

### General Description

In accordance with the present invention, there is provided a process or a method for adaptation of a highly attenuated recombinant VSV to tissue culture conditions by continuous passaging at low multiplicity of infection (MOI) in Vero cells or in any susceptible cell substrate. The multiple serial passaging process results in genotypic changes characterized by progressive accrual of a number of nucleotide substitutions throughout the viral genome. Most of these nucleotide substitutions result in amino acid (AA) substitutions in the VSV proteins. This process resulted in phenotypic adaptation of the virus, accompanied by substantial improvements in virus yield. Passage in Vero cells is continued until genotypic and phenotypic stability is achieved, usually in 10 to 15 serial passages (P10-P15). Further passaging of the virus beyond P15 showed few or no additional substitutions and did not result in further enhancement of virus yields. This process results in substantial improvement in manufacturing yield as well as enhanced manufacturing consistency. The adaptive mutations did not substantially affect the neurovirulence (NV) of the passaged virus when tested in the highly sensitive mouse intracranial NV animal model.

In accordance with U.S. Patent Publication No. 2007/0218078A1, a highly attenuated VSV vector expressing the HIV-1 gag gene was generated, rVSVN4CT1-gag1, which was made by combining three virus attenuating approaches: insertion of HIV-1 gag gene in the first position of the genome (gag1), thereby shifting all VSV genes from the 3′-promoter by one position, translocation of VSV N gene to the 4^{th} position (N4) and use of a VSV G with its cytoplasmic tail truncated to 1 amino acid (CT1) (Schnell, et al. The EMBO Journal 17:1289-1296, 1998). Compared to the prototypic rVSV, this vector displayed a marked increase in attenuation in vitro as characterized by smaller plaque phenotype, delayed growth kinetics and greatly reduced peak titers in cell culture. A similar pattern of attenuation was displayed when tested in a highly sensitive murine intracranial (IC) animal model, which showed differences in LD₅₀ of many orders of magnitude between the prototypic and attenuated VSV (Cooper et al, J. Virology, 82:207-229, 2008). In addition, compared to prototypic VSV, the attenuated virus displayed minimal to undetectable neuropathology in non-human primates. Although this vector induces potent immune responses in mice and macaques that were comparable to those obtained with the prototype vector, it replicates poorly in cell culture making it suboptimal for scale up and manufacturing.

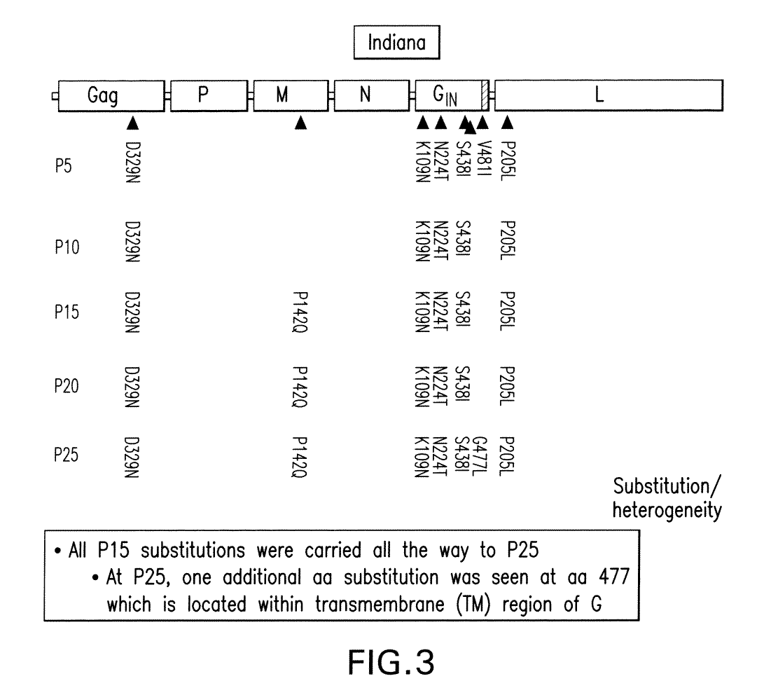

Based on the encouraging preclinical safety and immunogenicity profile, this vector would make a promising vector candidate for testing in humans. Two factors that are critical for testing of this vector in humans are the stability of the three attenuating mutations noted above and the increased manufacturing yields. In the present study, genetic stability studies were conducted, which consisted of serial passages of rVSV vectors in Vero cells. Viruses were passaged in Vero cells at low multiplicity of infection (MOI, 0.01). The data obtained from the viruses passaged 25 times revealed that while all three vector-attenuating mutations were retained, additional amino acids (AA) substitutions appeared as early as passage 5 (P5). As viruses were passaged further, mutations were fixed by passage 15 (P15). In addition, growth kinetics of the passaged viruses in Vero cells showed progressive improvement in virus yields up to P15, suggesting that accrual of AA substitutions represented continuing adaptation of virus to Vero cells. No further enhancement in virus yields was seen after P15. Results from the growth kinetics with passaged viruses indicated that the virus yield with P15 virus was 5- to 100-fold higher than that obtained with P0 (See FIGS. 3-6).

Accordingly, this invention demonstrates that P15 viruses exhibit genotypic and phenotypic stability and grow to levels suitable for large-scale manufacturing of clinical trial material suitable for toxicological and clinical evaluation.

### Suitable Cells for Growth/Propagation of VSV

Suitable host cells for use within the invention are capable of supporting a productive infection of the genetically modified attenuated VSV, and will permit expression of the requisite vectors and their encoded products necessary to support viral production. Examples of host cells for use in the methods of the present invention include, but are not limited to, Vero cells, baby hamster kidney cells (BHK), and human embryonic kidney (HEK) cells, such as 293 cells. Any other cell that is susceptible to infection with the genetically modified attenuated VSV strains or serotypes described herein, may be used in the methods of the invention.

### Production of VSV in a Mammalian Cell Culture

The production of VSV in mammalian cell culture is well known to one of skill in the art, and generally includes infecting the cell culture (host cell) with VSV, growing the VSV in cell culture and harvesting the cell culture at the appropriate time. Because VSV is secreted from the host cell into the media, the VSV product is collected from the cell culture fluid.

The production of VSV from mammalian cell culture employs suitable mammalian cell cultures used to propagate (or grow) VSV, which are known in the art. Such cell cultures include, but are not limited to, human embryonic kidney (HEK) cells such as HEK 293 cells, African green monkey kidney (AGMK) cells such as Vero cells, Chinese hamster ovary (CHO) cells and baby hamster kidney (BHK) cells.

Additionally, cell culture materials, methods and techniques are well known to one of skill in the art. For example, a recombinant VSV seed stock is used to infect a confluent host cell population or a host cell population at a certain density (e.g., a Vero cell culture) in a bioreactor at a given multiplicity of infection; the VSV is grown in cell culture for a given time and temperature; and the nascent VSV progeny harvested in the cell culture fluid. As defined hereinafter, the terms “culture fluid”, “cell culture fluid”, “cell culture media”, “media” and/or “bioreactor fluid” are used interchangeably, and refer to the media or solution in which the cell culture is grown.

Purification of VSV from a Mammalian Cell Culture

The processes for purifying VSV from cell culture fluid of a mammalian cell culture infected with VSV are generally known to one skilled in the art. For example, as described in Miller et al. (Protein Expression and Purification, Vol. 33, Issue 1, January 2004, pp. 92-103), culture supernatant containing the virus may be collected and subjected to low-speed centrifugation (eg. 1000×g) to remove cells and debris, followed by high speed centrifugation (eg. 100,000×g) over a 20% sucrose cushion to remove the virus.

Another method for purification of VSV from cell culture is described in U.S. Patent Publication 2007/0249019. Briefly, this procedure includes the steps of (a) primary clarification, (b) secondary clarification, (c) anion exchange membrane adsorption, (d) tangential flow filtration and (e) filtration. More particularly, such steps comprise (a) clarifying the cell culture fluid by low-speed centrifugation, (b) further clarifying the supernatant by filtration through a 0.2 to 0.45 micron filter, (c) purifying the VSV filtered solution on an anion exchange membrane adsorber, (d) buffer exchanging and concentrating the VSV by tangential flow filtration (TFF) and (e) a final filtration of the VSV retentate through a 0.2 to 0.22 micron filter. The purification process steps (a) through (e) above are performed at room temperature.

### Clarification Procedures

### Primary Clarification

Also described in U.S. Patent Publication 2007/0249019 is a method for primary clarification of the cell culture fluid to isolate and purify the virus. For example, the cell culture fluid of a mammalian cell culture infected with VSV may be clarified by low-speed centrifugation (or alternatively, by depth filtration) and the VSV recovered in the supernatant, also referred to herein as “primary clarification” of the cell culture fluid. In certain embodiments, primary clarification of the cell culture fluid is conducted at room temperature.

The centrifugation methods and equipment used in the primary clarification of the cell culture fluid are well known to one of skill in the art. As defined hereinafter, “low-speed” centrifugation is a centrifugation speed at or below 10,000 rpm.

As stated above, the cell culture fluid of a mammalian cell culture infected with VSV may be alternatively clarified by depth filtration (i.e., instead of low-speed centrifugation). Depth filtration can be used when low-speed centrifugation is omitted from primary clarification of step (a). Depth filtration (in contrast to surface filtration) generally refers to a “thick” filter that captures contaminants within its structure. Depth filtration materials and methods are well known to one of skill in the art. For example, the filter material is typically composed of a thick and fibrous cellulosic structure with inorganic filter aids such as diatomaceous earth particles embedded in the openings of the fibers. This filter material has a large internal surface area, which is key to particle capture and filter capacity. Such depth filtration modules contains pores of from 1.0 micron to 4.5 micron. Exemplary depth filtration modules include, but are not limited to, Whatman® Polycap™. HD modules (Whatman Inc.; Florham Park, N.J.), Sartorius Sartoclear™ P modules (Sartorius Corp.; Edgewood, N.Y.) and Millipore® Millistak+.® HC modules (Millipore; Billerica, Mass.). The cell culture fluid may be clarified via depth filtration (performed at room temperature) and the VSV is recovered in the filtrate.

### Secondary Clarification

Also described in U.S. Patent Publication 2007/0249019 is a method for secondary clarification of the cell culture fluid to isolate and purify the virus. After primary clarification via centrifugation (or depth filtration), the VSV supernatant (or filtrate) is further clarified by filtration, or microfiltration, through a 0.2 to 0.25 micron filter and recovery of the VSV in the filtered solution. The microfiltration may be performed at room temperature, as defined above. Filtration/Microfiltration media are available in a wide variety of materials and methods of manufacture, which are known to one of skill in the art. Exemplary microfiltration filter units include, but are not limited to, Millipore Millex.®.-GV filter units (Millipore; Billerica, Mass.), Millipore Millex.®-GP filter units, Pall Supor® filter units (Pall Corp.; East Hills, N.Y.), Sartorius Sartobran™ filter units (Sartorius Corp.; Edgewood, N.Y.) and Sartorius Sartopore™ 2 filter units. In certain embodiments, these filtration units posses filters of a size between 0.2 to 0.45 microns. The filtered VSV is recovered in the filtered solution.

### Anion Exchange Membrane Adsorption

Once the VSV has been recovered after clarification, the VSV may be further purified on an anion exchange membrane adsorber. Membrane adsorber materials are well known to one of skill in the art and available from vendors such as Sartorius Corp. (Edgewood, N.Y.), Pall Corp. (East Hills, N.Y.) and Sigma-Aldrich Corp. (St. Louis, Mo.). Exemplary anion exchange membrane adsorbers include, but are not limited to a Sartobind™ Q membrane adsorber (Sartorius Corp.) and a Mustang™ Q membrane adsorber (Pall Corp.). In one particular embodiment, the anion exchange membrane adsorber is a Pall Mustang™ Q membrane adsorber. In general, methods and buffers known from conventional ion exchange chromatography can be directly applied to membrane adsorber chromatography, which are known to one of skill in the art. In certain embodiments, the anion exchange membrane adsorber chromatography is performed at room temperature, as defined above.

Thus, the VSV may be purified via an anion exchange membrane adsorber, wherein the VSV filtered solution from the secondary clarification is loaded onto the anion exchange membrane adsorber equilibrated with a first pH buffered salt solution (also referred to as an “equilibration buffer” or VSV “binding buffer”). The VSV is eluted from the anion exchange membrane adsorber with a second pH buffered salt solution (“the elution buffer”) and the eluted VSV fractions are recovered (e.g., see U.S. Patent Publication 2007/0249019).

The first pH buffered salt solution or equilibration buffer may be an NaCl or KCL salt solution. The NaCl or KCl may be present in solution at an ionic strength between about at least 0.1 M to about 0.4 M, including fractional ionic strengths therebetween. The buffer solution may be a phosphate buffer, a N-2-Hydroxyethylpiperazine-N′-2-ethanesulfonic acid (HEPES) buffer or a Tris(hydroxymethyl)aminomethane (TRIS) buffer. These buffers may have a pH between about 6.0 to about 8.0.

The equilibration buffer may further comprise about 1% sucrose to about 5% sucrose. The second pH buffered salt solution (the “elution buffer”) may also comprise the same buffering components as the first (equilibration) buffer. The elution buffer may further comprise about 1% sucrose to about 5% sucrose.

To elute the VSV from the membrane, the salt (NaCl or KCl) concentration (ionic strength) of the elution buffer may be increased by linear gradient or in a single step elution process (also described in U.S. Patent Publication 2007/0249019). Both steps are equally effective at eluting VSV from the anion exchange membrane adsorber. The ionic strength of the NaCl in the second pH buffered salt solution should be between 0.5 M to 0.75 M.

The ionic strength of the NaCl in the second pH buffered salt solution should be linearly increased from 0.001 M to 0.75 M at an elution flow rate of about 10 CV/minute to 30 CV/minute.

### Tangential Flow Filtration (TFF)

Following VSV purification by anion exchange membrane adsorber chromatography, the VSV may be further purified by tangential flow filtration (TFF). In general, TFF is a pressure driven process that uses a membrane(s) to separate components in a liquid solution (or suspension), wherein a fluid (the feed flow) is pumped tangentially along the surface of the membrane and an applied pressure serves to force a “portion” of the fluid through the membrane to the filtrate side (of the membrane). TFF may be performed at room temperature. In this process, the buffer is exchanged and the VSV is concentrated. The TFF step may comprise concentrating the VSV recovered from the anion exchange membrane adsorption step at least 5 times, followed by at least one buffer exchange.

TFF materials (e.g., hollow fiber, spiral-wound, flat plate) and methods (e.g., ultrafiltration (UF), diafiltration (DF), microfiltration) are well known to one of skill in the art. The TFF membrane may have a 300 to 750 kDa molecular weight cutoff.

The buffer used in the buffer exchange of the TFF may be a phosphate buffer, HEPES buffer or TRIS buffer as described above. However, the buffer may have a concentration of about 5 mM to 15 mM, including mM concentrations therebetween. The buffer exchange buffer may further comprise 0.10 M to 0.20 M NaCl and 3.5% to 4.5% sucrose.

The VSV fractions from the anion exchange membrane adsorber purification may be pooled, and the pooled solution concentrated and the buffer exchanged by TFF using a hollow fiber TFF membrane cartridge with a molecular weight cut-off of about 750 kDa (GE Healthcare Bio-Sciences Corp.; Piscataway, N.J.).

### Filtration

In the method described in U.S. Patent Publication 2007/0249019, the last process step in virus purification is a final microfiltration of the VSV retentate from the TFF, wherein the retentate is filtered through a 0.2 to 0.25 micron filter, as described above for secondary clarification via microfiltration.

### Recombinant or Genetically Modified Vesicular Stomatitis Virus (VSV)

The VSV, as described herein, may be obtained and purified from mammalian cell culture by employing any of the purification methods described above. By “improved purity” is meant that the purified VSV is at least 90.0% free of cell culture protein and nucleic acid contaminants and preferably 99.0% to 99.8% free of cell culture protein and nucleic acid contaminants.

In particular embodiments, the VSV purified from cell culture fluid of a mammalian cell culture by any of the processes described above is a recombinant or genetically modified and/or attenuated VSV. Methods of producing recombinant RNA viruses, such as VSV, are well known and referred to in the art as “rescue” or “reverse genetics” methods. Exemplary rescue methods for VSV include, but are not limited to, the methods described in U.S. Pat. No. 6,033,886 and U.S. Pat. No. 6,168,943, each incorporated herein by reference. Additional techniques for conducting rescue of viruses, such as VSV, are described in U.S. Pat. No. 6,673,572 and WO 2004/113517, which are hereby incorporated by reference.

The VSV may be a VSV of a specified serotype. In certain embodiments, the purified VSV is an Indiana serotype, a New Jersey serotype, an Isfahan serotype, a Chandipura serotype, or other vesiculoviruses. In certain embodiments the VSV may contain sequences from more than one such serotype.

VSV vectors (and immunogenic compositions thereof often comprise one or more attenuating mutations within the VSV genome. In certain embodiments, the purified VSV has a genomic sequence comprising at least one mutation, which attenuates the pathogenicity of VSV. In other embodiments, the purified VSV has a genomic sequence comprising at least two mutations, which attenuate the pathogenicity of VSV. For example, an attenuated VSV may comprise two or more known attenuating mutations, such as the attenuating mutations set forth in International Application No. PCT/US2005/011499 (International Publication No. WO 2005/098009), and U.S. Patent Publication number 2007/0218078A1, incorporated herein by reference. For example, known VSV attenuating mutations include, but are not limited to, gene shuffling mutations (including gene shuffles of the VSV genes forming the VSV genome and designated N, P, M, G and L), G protein insertional mutations, G protein truncation mutations, temperature sensitive (ts) mutations (and other point mutations), non-cytopathic M gene mutations, G-stem mutations, ambisense RNA mutations and gene deletion mutations, each of which are set forth in detail in International Publication No. WO 2005/098009. Thus, in certain embodiments, the purified VSV comprises one or more attenuating mutations, including, without limitation, a temperature-sensitive (ts) mutation, a point mutation, a gene shuffling mutation, a G-stem mutation, a non-cytopathic M gene mutation, an ambisense RNA mutation, a truncated G gene mutation, a G gene insertion mutation and a gene deletion mutation.

In certain embodiments, the VSV purified by any of the purification processes described above has one or more mutations that result in virus attenuation, and one or more mutations that result in increased growth and an increase yield of virus from mammalian cells or cell lines. For example, the present invention provides a process or a method for adaptation of a highly attenuated VSV recombinant to tissue culture conditions by continuous passaging at low multiplicity of infection (MOI) in Vero cells or in any susceptible cell substrate. The multiple serial passaging process results in genotypic changes characterized by progressive accrual of a number of nucleotide (NT) substitutions throughout the viral genome. Most of these nucleotide substitutions result in amino acid (AA) substitutions in the VSV proteins. The process described herein resulted in phenotypic adaptation of the virus, accompanied by substantial improvements in virus yield. Passage in Vero cells was continued until genotypic and phenotypic stability was achieved, usually in 10 to 15 serial passages (P10-P15). Further passaging of the virus beyond P15 showed few or no additional substitutions and did not result in further enhancement of virus yields. This process results in substantial improvement in manufacturing yield as well as enhanced manufacturing consistency. The adaptive mutations did not substantially affect the neurovirulence (NV) of the passaged virus when tested in the highly sensitive mouse intracranial NV animal model.

One embodiment of the invention provides an isolated, genetically modified vesicular stomatitis virus (VSV) having at least one amino acid mutation in a region corresponding to at least one of the following positions:
the amino acids at positions 119 or 142 of the M protein;
the amino acids at positions 109, 224, 438, 477, or 481 of the G protein; and
the amino acids at positions 205, 220 or 1450 of the L protein.

In certain embodiments, the mutation is at either position 119 or 142 of the M protein or is at both positions 119 and 142 of the M protein. In other certain embodiments, the mutation of the amino acid at position 119 of the M protein is a T→N mutation and the mutation of the amino acid at position 142 of the M protein is a P→T mutation or a P→Q mutation.

In one embodiment, the mutation of the amino acids at position 109, 224, 438, 477 or 481 of the G protein is a K→N, N→T, S→I, A→V/G→L or V→I mutation, respectively.

In one embodiment, the mutation of the amino acid at position 205, 220 or 1450 of the L protein is P→L, K→E, or L→I, respectively.

In certain embodiments, the genetically modified and attenuated VSV as described herein has a genomic sequence comprising one or more foreign or heterologous (or foreign) polynucleotide sequences, such as a foreign RNA open reading frame (ORF). The heterologous polynucleotide sequences can vary as desired, and include, but are not limited to, a gene encoding a cytokine (such as an interleukin), a gene encoding T-helper epitope, a gene encoding a CTL epitope, a gene encoding an adjuvant and a gene encoding a co-factor, a gene encoding a restriction marker, a gene encoding a therapeutic protein or a protein of a different microbial pathogen (e.g. virus, bacterium, parasite or fungus), especially proteins capable of eliciting desirable immune responses. For example, the heterologous polynucleotide sequences encoding a protein of a different microbial pathogen may be one or more of a HIV gene, a HTLV gene, a SIV gene, a RSV gene, a PIV gene, a HSV gene, a CMV gene, an Epstein-Barr virus gene, a Varicella-Zoster virus gene, a mumps virus gene, a measles virus gene, an influenza virus gene, a poliovirus gene, a rhinovirus gene, a hepatitis A virus gene, a hepatitis B virus gene, a hepatitis C virus gene, a Norwalk virus gene, a togavirus gene, an alphavirus gene, a rubella virus gene, a rabies virus gene, a Marburg virus gene, an Ebola virus gene, a papilloma virus gene, a polyoma virus gene, a metapneumovirus gene, a coronavirus gene, a Vibrio cholerae gene, a Streptococcus pneumoniae gene, Streptococcus pyogenes gene, a Helicobacter pylori gene, a Streptococcus agalactiae gene, a Neisseria meningitidis gene, a Neisseria gonorrhoeae gene, a Corynebacterium diphtheriae gene, a Clostridium tetani gene, a Bordetella pertussis gene, a Haemophilus gene, a Chlamydia gene, and a Escherichia coli gene. In certain embodiments, the purified VSV comprises an HIV gene sequence, wherein the HIV sequence is selected from the group consisting of gag, env, pol, vif, nef, tat, vpr, rev or vpu. In one specific embodiment, the HIV gene is gag or env.

In certain other embodiments, the purified VSV contains both at least one attenuating mutation and at least one heterologous protein as described above. In other certain embodiments, the VSV immunogenic composition is a genetically modified VSV comprising two attenuating mutations and an orf encoding the HIV-1 gag protein. In one embodiment, the genetically modified VSV further comprises a nucleic acid molecule encoding the HIV gag protein, wherein the HIV gag protein has a mutation in at least one of the amino acids at position 165, 270, 329 or 348, wherein the mutation is S→G, L→S, D→N or T→K, respectively.

In other embodiments, the genetically modified VSV described herein encodes the HIV gag gene, wherein the gag gene is inserted into the VSV genome at position one (3′-gag₁-NPMGL-5′), position two (3′-N-gag₂-PMGL-5′), position three (3′-NP-gag₃-MGL-5′), position four (3′-NPM-gag₄-GL-5′), position five (3′-NPMG-gag₅-L-5′) or position six (3′-NPMGL-gag₆-5′). In other embodiments, the VSV described herein encodes the HIV env gene, wherein the env gene is inserted into the VSV genome at position one (3′-env₁-NPMGL-5′), position two (3′-N-env₂-PMGL-5′), position three (3′-NP-env₃-MGL-5′), position four (3′-NPM-env₄-GL-5′), position five (3′-NPMG-env₅-L-5′) or position six (3′-NPMGL-env₆-5′).

One of skill in the art would understand from the above description that a variety of genetic modifications in the VSV genome occurred during serial passage of the virus at low MOI in cell culture. These genetic modifications resulted in an increase in viral yield from infected cells. Moreover, there were no other genotypic or phenotypic changes to the virus and no alterations in virus attenuation. These genetic modifications proved to be of significant benefit in terms of increasing the viral yield to a level useful for scale-up in viral vector production.

The invention described hereinafter addresses a need in the art for vesicular stomatitis virus (VSV) vectors having significantly attenuated pathogenicity in mammals, particularly attenuated neuropathogenicity as revealed in animal neurovirulence models. As described above, VSV has many characteristics, which make it a suitable vector for immunogenic compositions. For example, VSV infection of humans is uncommon and is either asymptomatic or characterized by mild flu-like symptoms that resolve in three to eight days without complications, and as such, VSV is not considered a human pathogen. Other characteristics of VSV that render it an attractive vector include: (a) the ability to replicate robustly in cell culture; (b) the inability to either integrate into host cell DNA or undergo genetic recombination; (c) the existence of multiple serotypes, allowing the possibility for prime-boost immunization strategies; (d) foreign genes of interest can be inserted into the VSV genome and expressed abundantly by the viral transcriptase; (e) the development of a highly specialized system for the rescue of infectious virus from a cDNA copy of the virus genome (U.S. Pat. No. 6,033,886; U.S. Pat. No. 6,168,943) and (f) pre-existing immunity to VSV in the human population is infrequent.

### Attenuated Vesicular Stomatitis Viruses

In certain embodiments, an attenuated VSV for use in the present invention comprises one or more mutations from the classes of mutations listed below. Moreover, these attenuated viruses are further genetically modified using the methods of the present invention, eg. serial passage for about 5 to 15 times using a low MOI. This method results in retention of the attenuated genotype and phenotype, yet provides for further genetic modifications as described herein, which result in a VSV that is better adapted for growth in cell culture. The higher virus yields achieved by use of these viruses makes them excellent candidates for vector production.

### A. Vesicular Stomatitis Virus Mutation Classes

In one embodiment, a genetically modified VSV vector of the invention comprises at least two different classes of mutations in its genome. As noted previously, the terms “mutation class”, “mutation classes” or “classes of mutation” are used interchangeably, and refer to mutations known in the art, when used singly, to attenuate VSV. For example, a “mutation class” of the invention includes, but is not limited to, a VSV temperature-sensitive N gene mutation (hereinafter, “N₍ₜₛ₎”), a temperature-sensitive L gene mutation (hereinafter, “L₍ₜₛ₎”), a point mutation, a G-stem mutation (hereinafter, “G₍ₛₜₑₘ₎”), a non-cytopathic M gene mutation (hereinafter, “M_{(ncp)}”), a gene shuffling or rearrangement mutation, a truncated G gene mutation (hereinafter, “G_{(ct)}”), an ambisense RNA mutation, a G gene insertion mutation, a gene deletion mutation and the like. As defined hereinafter, a “mutation” includes mutations known in the art as insertions, deletions, substitutions, gene rearrangement or shuffling modifications.

Furthermore, as noted previously, the term “synergistic” attenuation refers to a level of VSV attenuation, which is greater than additive. For example, a synergistic attenuation of VSV comprises combining at least two classes of mutation in the same VSV genome, thereby resulting in a reduction of VSV pathogenicity much greater than an additive attenuation level observed for each VSV mutation class alone. Thus, in certain embodiments, a synergistic attenuation of VSV is defined as a LD₅₀ at least greater than the additive attenuation level observed for each mutation class alone (i.e., the sum of the two mutation classes), wherein attenuation levels (i.e., the LD₅₀) are determined in a small animal neurovirulence model.

By way of a non-limiting example, if equation (1) describes an “additive attenuation” of VSV:

Δa_{LD50}+Δb_{LD50}=x_{LD50}; (1)

wherein Δa_{LD50} is the LD₅₀ of a VSV having a first mutation class in its genome, Δb_{LD50} is the LD₅₀ of a VSV having a second mutation class in its genome and x_{LD50} is the sum of Δa_{LD50} and Δb_{LD50}; then a VSV “synergistic attenuation” of the invention, having a LD₅₀ at least greater than the additive attenuation level observed for each mutation class alone, is described by equation (2):

Δa,b_{LD50}>(Δa_{LD50}+Δb_{LD50}); (2)

wherein Δa,b_{LD50} is the LD₅₀ of a VSV having a combination of two mutation classes in its genome, Δa_{LD50} is the LD₅₀ of a VSV having a first mutation class in its genome and Δb_{LD50} is the LD₅₀ of a VSV having a second mutation class in its genome. Thus, in certain embodiments, the synergy of VSV attenuation (i.e., two mutation classes in the same VSV genome) is described relative to the LD₅₀ of two VSV constructs (each VSV construct having a single mutation class in its genome), wherein the synergistic attenuation of the VSV having two mutation classes in its genome is defined as a LD₅₀ at least greater than the additive LD₅₀ of the two VSV constructs having a single mutation class in their genome.

In certain other embodiments, the synergy of VSV attenuation is described relative to the LD₅₀ of a wild-type VSV. Thus, in one embodiment, a synergistic attenuation of VSV is defined as a LD₅₀ that is at least greater than the LD₅₀ of wild-type VSV, wherein the LD₅₀ is determined in an animal neuroviruience model. In one embodiment, a synergistic attenuation of VSV is defined as a LD₅₀ that is at least 10-fold greater than the LD₅₀ of wild-type VSV, wherein the LD₅₀ is determined in an animal neurovirulence model. In another embodiment, a synergistic attenuation of VSV is defined as a LD₅₀ that is at least 100-fold greater than the LD₅₀ of wild-type VSV, wherein the LD₅₀ is determined in an animal neurovirulence model. In another embodiment, a synergistic attenuation of VSV is defined as a LD₅₀ that is at least 1,000-fold greater than the LD₅₀ of wild-type VSV, wherein the LD₅₀ is determined in an animal neurovirulence model. In yet other embodiments, a synergistic attenuation of VSV is defined as a LD₅₀ that is at least 10,000-fold greater than the LD₅₀ of wild-type VSV, wherein the LD₅₀ is determined in an animal neurovirulence model. In certain other embodiments, a synergistic attenuation of VSV is defined as a LD₅₀ that is at least 100,000-fold greater than the LD₅₀ of wild-type VSV, wherein the LD₅₀ is determined in an animal neurovirulence model. The determination of a 50% lethal dose (LD₅₀) for a particular VSV vector is readily determined by a person of skill in the art using known testing methods and animal models.

### Gene Shuffling Mutations

In certain embodiments, a genetically modified VSV of the invention comprises a gene shuffling mutation in its genome. As defined herein, the terms “gene shuffling”, “shuffled gene”, “shuffled”, “shuffling”, “gene rearrangement” and “gene translocation” are used interchangeably, and refer to a change (mutation) in the order of the wild-type VSV genome. As defined herein, a wild-type VSV genome has the following gene order: 3′-NPMGL-5′.

It is known in the art, that the position of a VSV gene relative to the 3′ promoter determines the level of expression and virus attenuation (U.S. Pat. No. 6,596,529 and Wertz et al., 1998, each specifically incorporated herein by reference). There is a gradient of expression, with genes proximal to the 3′ promoter expressed more abundantly than genes distal to the 3′ promoter. The nucleotide sequences encoding VSV G, M, N, P and L proteins are known in the art (Rose and Gallione, 1981; Gallione et al., 1981). For example, U.S. Pat. No. 6,596,529 describes gene shuffling mutations in which the gene for the N protein is translocated (shuffled) from its wild-type promoter-proximal first position to successively more distal positions on the genome, in order to successively reduce N protein expression (e.g., 3′-PNMGL-5′,3′-PMNGL-5′,3′-PMGNL-5′, referred to as N2, N3 and N4, respectively). Thus, in certain embodiments, a genetically modified VSV comprises a gene shuffling mutation in its genome. In one class of mutation, in one particular embodiment, a genetically modified VSV comprises a gene shuffling mutation comprising a translocation of the N gene (e.g., 3′-PNMGL-5′ or 3′-PMNGL-5′).

It should be noted herein, that the insertion of a foreign nucleic acid sequence (e.g., HIV gag) into the VSV genome 3′ to any of the N, P, M, G or L genes, effectively results in a “gene shuffling mutation” as defined above. For example, when the HIV gag gene is inserted into the VSV genome at position one (e.g., 3′-gag₁-NPMGL-5′), the N, P, M, G and L genes are each moved from their wild-type positions to more distal positions on the genome. Thus, in certain embodiments of the invention, a gene shuffling mutation includes the insertion of a foreign nucleic acid sequence into the VSV genome 3′ to any of the N, P, M, G or L genes (e.g., 3′-gag₁-NPMGL-5′, 3′-N-gag₂-PMGL-5′, 3′-NP-gag₃-MGL-5′, etc.)

### G Protein Insertion and Truncation Mutants

In certain other embodiments, a genetically modified VSV of the invention comprises a mutated G gene, wherein the encoded G protein is truncated at its cytoplasmic domain (carboxy-terminus), also referred to as the “cytoplasmic tail region” of the G protein. It is known in the art that G gene mutations which truncate the carboxy-terminus of the cytoplasmic domain influence VSV budding and attenuate virus production (Schnell, et al. The EMBO Journal 17(5):1289-1296, 1998; Roberts, et al. J Virol, 73:3723-3732, 1999). The cytoplasmic domain of wild-type VSV G protein comprises twenty-nine amino acids (RVGIHLCIKLKHTKKRQIYTDIEMNRLGK—COOH; SEQ ID NO:1).

In certain embodiments, a truncated VSV G gene of the invention encodes a G protein in which the last twenty-eight carboxy-terminal amino acid residues of the cytoplasmic domain are deleted (retaining only arginine from the twenty-nine amino acid wild-type cytoplasmic domain of SEQ ID NO:1). In certain other embodiments, a truncated VSV G gene of the invention encodes a G protein in which the last twenty carboxy-terminal amino acid residues of the cytoplasmic domain are deleted (relative to the twenty-nine amino acid wild-type cytoplasmic domain of SEQ ID NO:1).

In certain other embodiments, a truncated VSV G gene of the invention encodes a G protein comprising a single amino acid in its cytoplasmic domain (cytoplasmic tail region), wherein the single amino acid is any naturally occurring amino acid. In still other embodiments, a truncated VSV G gene of the invention encodes a G protein comprising nine amino acids in its cytoplasmic domain (cytoplasmic tail region), wherein the nine amino acids are any naturally occurring amino acids. In certain other embodiments, a mutated VSV gene of the invention encodes a G protein containing an insertion representing a foreign epitope. Such mutants are known in the art (e.g., see Schlehuber and Rose, 2003).

As defined herein, a G gene mutant encoding a G protein in which the last twenty-eight carboxy-terminal amino acid residues of the cytoplasmic domain are deleted, relative to the wild-type sequence of SEQ ID NO: 1, is designated “G_{(ct-1)}”, or simply as “CT1”, wherein the cytoplasmic domain of the G_{(ct-1)} has an amino acid sequence of (R—COOH). As defined herein, a G gene mutant encoding a G protein in which the last twenty carboxy-terminal amino acid residues of the cytoplasmic domain are deleted, relative to the wild-type sequence of SEQ ID NO:1, is designated “G_{(ct-9)}”, wherein the cytoplasmic domain of the G_{(ct-9)}, or simply as “CT9”, has an amino acid sequence of (RVGIHLCIK—COOH; SEQ ID NO:2). Thus, in certain embodiments of the invention, a genetically modified VSV of the invention comprises a mutated G gene, wherein the encoded G protein is a G_{(ct-1)} or G_{(ct-9)}.

### Temperature Sensitive and Other Point Mutations

A VSV “temperature-sensitive” (“ts”) mutation, as defined hereinafter, is a mutation in the VSV genome which restricts VSV growth at a non-permissive temperature. For example, a VSV ts mutant of the invention grows normally and to high titer at the permissive temperature (e.g., 31° C.), but their growth or reproduction is restricted at non-permissive temperatures (e.g., 37° C. or 39° C.). The generation of ts mutants by chemical and site directed mutagenesis are well known in the art (e.g., see Pringle, 1970; Li et al., 1988); and numerous ts mutants have been characterized and described (e.g., see Flamand and Pringle, 1971; Flamand and Bishop, 1973; Printz and Wagner, 1971; Gopalakrishna and Lenard, 1985; Pringle et al., 1981; Morita et al., 1987; Li et al., 1988; Rabinowitz et al., 1977; Lundh et al., 1988; Dal Canto et al., 1976; Rabinowitz et al., 1976). In certain embodiments, a genetically modified VSV of the invention comprises a ts mutation in its genome, wherein the ts mutation is one or more mutations of a nucleic acid sequence encoding the G, M, N, P or L protein.

As defined herein, a ts mutation of any one the VSV G, M, N, P or L genes is a separate “mutation class” of the invention. For example, in certain embodiments of the invention, a genetically modified VSV comprising at least two different classes of mutations in its genome (wherein the two mutations synergistically attenuate VSV pathogenicity) comprises one or more ts N gene mutation(s) (hereinafter, “N₍ₜₛ₎”) as a first class of mutation and one or more ts L gene mutation(s) (hereinafter, “L₍ₜₛ₎”) as a second class of mutation. As a non-limiting example, a genetically modified VSV comprising a genome such as 3′-N₍ₜₛ₎PMGL₍ₜₛ₎-5′ comprises two classes of mutations (i.e., (1) an N₍ₜₛ₎ gene mutation and (2) an L₍ₜₛ₎ gene mutation) and a genetically modified VSV comprising a genome such as 3′-gag₁-N₍ₜₛ₎PMGL₍ₜₛ₎-5′ comprises three classes of mutations (i.e., (1) an N₍ₜₛ₎ gene mutation, (2) an L₍ₜₛ₎ gene mutation and (3) by way gag₁ insertion, a gene shuffling mutation).

In certain other embodiments, a genetically modified VSV of the invention comprises a point mutation in its genome, wherein the point mutation is one or more mutations of a nucleic acid sequence encoding the G, M, N, P or L protein, wherein the mutation confers an attenuating phenotype such as cold-adaptation, decreased fusion or cytopathogenic efficiency (e.g., see Fredericksen and Whitt, 1998; Ahmed and Lyles, 1997). For example, Fredericksen and Whitt (1998) describe three attenuating point mutations of the G gene (e.g., D137-L, E139-L or DE-SS) which have a shifted pH threshold for fusion activity. Ahmed and Lyles (1997) described an attenuating point mutation of the M gene (N163D) that was highly defective in inhibition of host gene expression and was turned over more rapidly than wild-type M protein. Thus, in certain embodiments, a genetically modified VSV of the invention comprises one or more point mutations in its genome.

### Non-Cytopathic M Gene Mutations

In certain other embodiments, a genetically modified VSV of the invention comprises a non-cytopathic mutation in the M gene. The VSV (Indiana serotype) M gene encodes a 229 amino acid M (matrix) protein, wherein the first thirty amino acids of the NH₂-terminus comprise a PPPY motif. It was demonstrated by Jayakar et al. (J. Virology, 74: 9818-27, (2000)), that mutations in the PPPY motif (e.g., APPY, AAPY, PPAY, APPA, MPA and PPPA) reduce virus yield by blocking a late stage in virus budding. Thus, in certain embodiments, a genetically modified VSV of the invention comprises a non-cytopathic mutation in the M gene, wherein the mutation is in the PPPY motif of the encoded M protein.

It has recently been reported that the M mRNA further encodes two additional proteins, referred to as M2 and M3 (Jayakar and Whitt, J. Virology, 76:8011-18, 2002). The M2 and M3 proteins are synthesized from downstream methionines in the same reading frame that encodes the 229 amino acid M protein (referred to as M1), and lack the first thirty-two (M2 protein) or fifty (M3 protein) amino acids of the M1 protein. It has been observed that cells infected with a recombinant VSV that expresses the M protein, but not M2 and M3, exhibit a delayed onset of cytopathic effect (in certain cell types), yet produce a normal virus yield. Thus, in certain embodiments, a genetically modified VSV of the invention comprises a non-cytopathic mutation in the M gene, wherein the M gene mutation results in a virus that does not express the M2 or M3 protein (e.g., see Jayakar and Whitt, 2002).

### G-Stem Mutations

In certain embodiments, a genetically modified VSV of the invention comprises a mutation in the G gene, wherein the encoded G protein has a mutation in the membrane-proximal stem region of the G protein ectodomain, referred to as G-stem protein. The G-stem region comprises amino acid residues 421 through 462 of the G protein. Recent studies have demonstrated the attenuation of VSV via insertion and/or deletion (e.g., truncation) mutations in the G-stem of the G protein (Robinson and Whitt, J. Virol., 74, 2239-46, 2000; Jeetendra et al., J. Virol, 76, 12300-311, 2002; Jeetendra et al., J. Virol, 77, 12807-18, 2003). Thus, in certain embodiments, a genetically modified VSV comprises a G-stem insertion, deletion, substitution or a combination thereof. In one particular embodiment, a genetically modified VSV vector of the invention comprising a G-stem mutation (and immunogenic compositions thereof, comprises a genome of 3′-gag₁-NPMG₍ₛₜₑₘ₎L-5′.

### Ambisense RNA Mutations

In certain embodiments, a genetically modified VSV of the invention comprises an ambisense RNA mutation, in which the 5′ antigenome promoter (AGP) is replaced with a copy of the 3′ genome promoter (GP). The 5′ AGP of VSV, as well as other nonsegmented, negative strand RNA viruses, acts as a strong replication promoter while the 3′ GP acts as a transcription promoter and a weak replication promoter. In the normal course of VSV infection, there is a 3- to 4-fold predominance of genome copies over antigenome copies; this ratio is even higher for rabies virus, another member of the Rhabdovirus family (Finke and Conzelmann, J. Virology, 73(5):3818-25, 1999). Previous work with rabies virus demonstrated that replacing the 5′ AGP with a copy of the GP (known as an ambisense RNA mutation) led to equal levels of genome and antigenome RNA copies within infected cells. In addition, a foreign gene was expressed from the copy of the GP placed at the 5′ end of the genome. When serially passaged in cultured cells, the rabies virus containing the ambisense RNA mutation consistently replicated to 10- to 15-fold lower titers than a recombinant wild type rabies virus (Finke and Conzelmann, J. Virology, 73(5):3818-25, 1999). Such a mutation is used in VSV vectors to both attenuate the virus replication and express foreign genes. Thus, in certain embodiments, a genetically modified VSV comprises an ambisense RNA mutation.

### Gene Deletions

In certain other embodiments, a genetically modified VSV of the invention comprises a virus in which a VSV gene (such as G or M) is deleted from the genome. For example, Roberts et al. (J. Virol., 73, 3723-32, 1999) described a VSV vector in which the entire gene encoding the G protein was deleted (AG) and substituted with influenza hemagglutinin (HA) protein, wherein the VSV vector (AG-HA) demonstrated attenuated pathogenesis.

### B. Methods for Generating Further Genetic Modifications of Attenuated VSV

In certain embodiments, the invention is directed to a genetically modified VSV comprising at least two different classes of mutations set forth below. Any of these genetically modified and attenuated VSV may be further genetically modified by the methods described in the present invention. That is, any of these attenuated VSV may be serially passaged in susceptible cells or cell lines for about 5 to 15 times at a low multiplicity of infection ranging from about 0.001 to about 0.1 PFU per cell and their genomes analyzed for the presence of at least one of the mutations described herein.

Accordingly, in one aspect of the invention, a method is provided for adapting a virus for growth in cell culture comprising the following steps:
a) infecting the cell culture with the virus at a low multiplicity-of-infection (MOI) ranging from about 0.001 to about 0.1 plaque forming units (PFU) per cell;
b) harvesting the cell culture medium containing the virus;
c) clarifying the cell culture medium;
d) freezing the cell culture medium; and
e) repeating steps a) through d) for about 5 to about 15 times.

The use of this method results in a 5 to 100 fold increase in virus production/yield and an increase in the stability of the virus genotype and phenotype characteristics, as compared to the virus production or yield using the VSV that has not been passaged at a low MOI for about 5 to 15 times at a low MOI of about 0.001 to about 0.1 PFU per cell.

The use of such method results in a genetically modified vesicular stomatitis virus (VSV) having at least one amino acid mutation in a region corresponding to at least one of the following positions:
the amino acids at positions 119 or 142 of the M protein;
the amino acids at positions 109, 224, 438, 477, or 481 of the G protein; and
the amino acids at positions 205, 220 or 1450 of the L protein.

In one embodiment, the genetically modified VSV produced by the methods of the invention has a mutation that comprises a conservative or non-conservative amino acid change.

In one embodiment, the genetically modified VSV produced by the methods of the invention has a mutation at either position 119 or 142 of the M protein or at both positions 119 and 142 of the M protein. In one embodiment, the mutation of the amino acid at position 119 of the M protein is a T→N mutation and the mutation of the amino acid at position 142 of the M protein is a P→T mutation or a P→Q mutation.

In one embodiment, the genetically modified VSV produced by the methods of the invention has a mutation of the amino acids at position 109, 224, 438, 477 or 481 of the G protein that is a K→N, N→T, S→I, A→V/G→L or V→I mutation, respectively.

In one embodiment, the genetically modified VSV produced by the methods of the invention has a mutation of the amino acid at position 205, 220 or 1450 of the L protein that is P→L, K→E, or L→I, respectively.

In one embodiment, the genetically modified VSV produced by the methods of the invention further comprises a nucleic acid molecule encoding the HIV gag protein, wherein the HIV gag protein has a mutation in at least one of the amino acids at position 165, 270, 329 or 348, wherein the mutation is S→G, L→S, D→N or T→K, respectively.

In one embodiment, the mutations noted above in the genetically modified VSV produced by the methods of the invention result in increased stability of the virus genotype and/or phenotype and further result in increased yield in virus production from a cell infected with the genetically modified VSV.

In one embodiment, the genetically modified VSV produced by the methods of the invention further comprises at least two other mutations in its genome, selected from any of those described above. In one embodiment, the mutations may be selected from the group consisting of a temperature-sensitive mutation, a point mutation, a gene shuffling mutation, a G-stem mutation, a non-cytopathic M gene mutation, an ambisense RNA mutation, a truncated G gene mutation, a G gene insertion mutation and a gene deletion mutation.

In one embodiment, the methods described herein utilize a virus that is an attenuated virus. In one embodiment, the methods for producing the genetically modified virus is adapted for large scale production of a viral vector or immunogenic composition. In one embodiment, the method results in a 5 to 100 fold higher yield of virus compared to that obtained with a virus strain that has not been passaged for about 5 to 15 times at a low multiplicity of infection ranging from about 0.001 to about 0.1 plaque forming units per cell. In one embodiment, the method described above allows for maintaining any pre-existing mutation(s) associated with virus attenuation. The pre-existing mutation(s) associated with virus attenuation may be selected from a temperature-sensitive mutation, a point mutation, a gene shuffling mutation, a G-stem mutation, a non-cytopathic M gene mutation, an ambisense RNA mutation, a truncated G gene mutation, a G gene insertion mutation and a gene deletion mutation. In one embodiment, the method allows for maintaining a low neurovirulence profile associated with virus attenuation. In one embodiment, the attenuated virus used in the methods described above is a strain of vesicular stomatitis virus (VSV).

### C. Recombinant Vesicular Stomatitis Virus Vectors

In certain embodiments, the invention provides a recombinant VSV vector comprising at least two different classes of mutations in its genome and at least one foreign RNA sequence inserted as a separate transcriptional unit into or replacing a region of the VSV genome non-essential for replication. This recombinant VSV vector may be further genetically modified to contain at least one of the modifications described above, which results in adaptation of the virus to growth in cell culture, thus increasing the yield of virus per cell.

Methods of producing recombinant RNA virus are referred to in the art as “rescue” or “reverse genetics” methods. Exemplary rescue methods for VSV are described in U.S. Pat. No. 6,033,886, U.S. Pat. No. 6,596,529 and WO 2004/113517, each incorporated herein by reference. The transcription and replication of negative-sense, single stranded, non-segmented, RNA viral genomes are achieved through the enzymatic activity of a multimeric protein complex acting on the ribonucleoprotein core (nucleocapsid). Naked genomic RNA cannot serve as a template. Instead, these genomic sequences are recognized only when they are entirely encapsidated by the N protein into the nucleocapsid structure. It is only in that context that the genomic and antigenomic terminal promoter sequences are recognized to initiate the transcriptional or replication pathways.

A cloned DNA equivalent of the VSV genome is placed between a suitable DNA-dependent RNA polymerase promoter (e.g., the T7 RNA polymerase promoter) and a self-cleaving ribozyme sequence (e.g., the hepatitis delta ribozyme), which is inserted into a suitable transcription vector (e.g., a propagatable bacterial plasmid). This transcription vector provides the readily manipulable DNA template from which the RNA polymerase (e.g., T7 RNA polymerase) can faithfully transcribe a single-stranded RNA copy of the VSV antigenome (or genome) with the precise, or nearly precise, 5′ and 3′ termini. The orientation of the VSV genomic DNA copy and the flanking promoter and ribozyme sequences determine whether antigenome or genome RNA equivalents are transcribed. Also required for rescue of new VSV progeny are the VSV-specific trans-acting support proteins needed to encapsidate the naked, single-stranded VSV antigenome or genome RNA transcripts into functional nucleocapsid templates: the viral nucleocapsid (N) protein, the polymerase-associated phosphoprotein (P) and the polymerase (L) protein. These proteins comprise the active viral RNA-dependent RNA polymerase which must engage this nucleocapsid template to achieve transcription and replication.

Thus, a genetically modified and attenuated VSV of the invention, comprising at least two different classes of mutations in its genome (e.g., see Section A), is produced according to rescue methods known in the art. For example, a genetically modified VSV vector comprising at least two different classes of mutations in its genome is generated using (1) a transcription vector comprising an isolated nucleic acid molecule which comprises a polynucleotide sequence encoding a genome or antigenome of a VSV and (2) at least one expression vector which comprises at least one isolated nucleic acid molecule encoding the trans-acting N, P and L proteins necessary for encapsidation, transcription and replication; in a host cell under conditions sufficient to permit the co-expression of these vectors and the production of the recombinant VSV. Any suitable VSV strain or serotype may be used according to the present invention, including, but not limited to, VSV Indiana, VSV New Jersey, VSV Chandipura, VSV Glasgow, and the like.

In addition to polynucleotide sequences encoding attenuated forms of VSV, the polynucleotide sequence may also encode one or more heterologous (or foreign) polynucleotide sequences or open reading frames (ORFs). The heterologous polynucleotide sequences can vary as desired, and include, but are not limited to, a co-factor, a cytokine (such as an interleukin), a T-helper epitope, a CTL epitope, a restriction marker, an adjuvant, or a protein of a different microbial pathogen (e.g. virus, bacterium, parasite or fungus), especially proteins capable of eliciting desirable immune responses. In certain embodiments, a heterologous ORF contains an HIV gene (e.g., gag, env, pol, vif, net, tat, vpr, rev or vpu). In one particular embodiment, the HIV gene is gag, wherein the gag gene is inserted into the VSV genome at position one (3′-gag₁-NPMGL-5′) or at position five (3′-NPMG-gag₅-L-5′). In another embodiment, the heterologous polynucleotide sequence further encodes a cytokine, such as interleukin-12, which are selected to improve the prophylactic or therapeutic characteristics of the recombinant VSV.

In certain embodiments, a genetically modified and attenuated VSV of the invention is mutated by conventional means, such as chemical mutagenesis. For example, during virus growth in cell cultures, a chemical mutagen is added, followed by: (a) selection of virus that has been subjected to passage at suboptimal temperature in order to select temperature-sensitive and/or cold adapted mutations, (b) identification of mutant virus that produce small plaques in cell culture, and (c) passage through heterologous hosts to select for host range mutations. In other embodiments, attenuating mutations comprise making predetermined mutations using site-directed mutagenesis and then rescuing virus containing these mutations. As set forth previously, a genetically modified VSV of the invention comprises at least two classes of mutation in its genome. In certain embodiments, one or more classes of mutation further comprises multiple mutations, such as a G-stem mutation class having a double mutation (e.g., a deletion, insertion, substitution, etc.), a triple mutation and the like. These attenuated VSV vectors are then screened for attenuation of their virulence in an animal model.

The typical (although not necessarily exclusive) circumstances for rescue include an appropriate mammalian cell milieu in which T7 polymerase is present to drive transcription of the antigenomic (or genomic) single-stranded RNA from the viral genomic cDNA-containing transcription vector. Either co-transcriptionally or shortly thereafter, this viral antigenome (or genome) RNA transcript is encapsidated into functional templates by the nucleocapsid protein and engaged by the required polymerase components produced concurrently from co-transfected expression plasmids encoding the required virus-specific trans-acting proteins. These events and processes lead to the prerequisite transcription of viral mRNAs, the replication and amplification of new genomes and, thereby, the production of novel VSV progeny, i.e., rescue.

The transcription vector and expression vector are typically plasmid vectors designed for expression in the host cell. The expression vector which comprises at least one isolated nucleic acid molecule encoding the trans-acting proteins necessary for encapsidation, transcription and replication expresses these proteins from the same expression vector or at least two different vectors. These vectors are generally known from the basic rescue methods, and they need not be altered for use in the improved methods of this invention.

Additional techniques for conducting rescue of viruses such as VSV, are described in U.S. Pat. No. 6,673,572 and U.S. Published Patent Application US20060153870, which are hereby incorporated by reference.

The host cells used in the rescue of VSV are those which permit the expression from the vectors of the requisite constituents necessary for the production of recombinant VSV. Such host cells can be selected from a prokaryotic cell or a eukaryotic cell, and preferably a vertebrate cell. In general, host cells are derived from a human cell, such as a human embryonic kidney cell (e.g., 293). Vero cells, as well as many other types of cells are also used as host cells. The following are non-limiting examples of suitable host cells: (1) Human Diploid Primary Cell Lines (e.g. WI-38 and MRC5 cells); (2) Monkey Diploid Cell Line (e.g. FRhL-Fetal Rhesus Lung cells); (3) Quasi-Primary Continues Cell Line (e.g. AGMK-African green monkey kidney cells); (4) Human 293 cells and (5) other potential cell lines, such as, CHO, MDCK (Madin-Darby Canine Kidney), primary chick embryo fibroblasts. In certain embodiments, a transfection facilitating reagent is added to increase DNA uptake by cells. Many of these reagents are known in the art (e.g., calcium phosphate). Lipofectace (Life Technologies, Gaithersburg, Md.) and Effectene (Qiagen, Valencia, Calif.) are common examples. Lipofectace and Effectene are both cationic lipids. They both coat DNA and enhance DNA uptake by cells. Lipofectace forms a liposome that surrounds the DNA while Effectene coats the DNA but does not form a liposome. Alternatively, the plasmid DNA uptake can also be enhanced by electroporation of the cells, whereby a high voltage current is applied across cuvette containing cells and DNA for milliseconds.

The rescued attenuated VSV is then tested for its desired phenotype (temperature sensitivity, cold adaptation, plaque morphology, and transcription and replication attenuation), first by in vitro means. The mutations are also tested using a minireplicon system where the required trans-acting encapsidation and polymerase activities are provided by wild-type or modified helper viruses, or by plasmids expressing the N, P and different L genes harboring gene-specific attenuating mutations. The attenuated VSV is also tested in vivo for synergistic attenuation in an animal neurovirulence model. For example, mouse and/or ferret models are established for detecting neurovirulence. Briefly, groups of ten mice are injected intra-cranially (IC) with each of a range of virus concentrations that span the anticipated LD₅₀ dose (a dose that is lethal for 50% of animals). For example, IC inoculations with virus at 10², 10³, 10⁴ and 10⁵ pfu are used where the anticipated LD₅₀ for the virus is in the range 10³-10⁴ pfu. Virus formulations are prepared by serial dilution of purified virus stocks in PBS. Mice are then injected through the top of the cranium with the requisite dose, in 50-100 μl of PBS. Animals are monitored daily for weight loss, morbidity and death. The LD₅₀ for a virus vector is then calculated from the cumulative death of mice over the range of concentrations tested.

### Heterologous Nucleic Acid Sequences and Antigens

In certain embodiments, the invention provides synergistically attenuated and genetically modified VSV (using the serial passage methods of the present invention at a low MOI) further comprising a foreign RNA sequence as a separate transcriptional unit inserted into or replacing a site of the genome nonessential for replication, wherein the foreign RNA sequence (which is in the negative sense) directs the production of a protein capable of being expressed in a host cell infected by VSV. This recombinant genome is originally produced by insertion of foreign DNA encoding the protein into the VSV cDNA. In certain embodiments, any DNA sequence which encodes an immunogenic antigen, which produces prophylactic or therapeutic immunity against a disease or disorder, when expressed as a fusion or non-fusion protein in a recombinant synergistically attenuated VSV of the invention, alone or in combination with other antigens expressed by the same or a different VSV, is isolated and incorporated in the VSV vector for use in the immunogenic compositions of the present invention.

In certain embodiments, expression of an antigen by a synergistically attenuated and further genetically modified (using the methods of the invention) recombinant VSV induces an immune response against a pathogenic microorganism. For example, an antigen may display the immunogenicity or antigenicity of an antigen found on bacteria, parasites, viruses, or fungi which are causative agents of diseases or disorders. In one embodiment, antigens displaying the antigenicity or immunogenicity of an antigen of a human pathogen or other antigens of interest are used.

To determine immunogenicity or antigenicity by detecting binding to antibody, various immunoassays known in the art are used, including but not limited to, competitive and non-competitive assay systems using techniques such as radioimmunoassays, ELISA (enzyme linked immunosorbent assay), “sandwich” immunoassays, immunoradiometric assays, gel diffusion precipitin reactions, immunodiffusion assays, in situ immunoassays (using colloidal gold, enzyme or radioisotope labels, for example), western blots, immunoprecipitation reactions, agglutination assays (e.g., gel agglutination assays, hemagglutination assays), complement fixation assays, immunofluorescence assays, protein A assays, and immunoelectrophoresis assays, neutralization assays, etc. In one embodiment, antibody binding is measured by detecting a label on the primary antibody. In another embodiment, the primary antibody is detected by measuring binding of a secondary antibody or reagent to the primary antibody. In a further embodiment, the secondary antibody is labeled. Many means are known in the art for detecting binding in an immunoassay. In one embodiment for detecting immunogenicity, T cell-mediated responses are assayed by standard methods, e.g., in vitro or in vivo cytotoxicity assays, tetramer assays, elispot assays or in vivo delayed-type hypersensitivity assays.

Parasites and bacteria expressing epitopes (antigenic determinants) that are expressed by synergistically attenuated VSV (wherein the foreign RNA directs the production of an antigen of the parasite or bacteria or a derivative thereof containing an epitope thereof) include but are not limited to those listed in Table 1.

In another embodiment, the antigen comprises an epitope of an antigen of a nematode, to protect against disorders caused by such worms. In another embodiment, any DNA sequence which encodes a Plasmodium epitope, which when expressed by a recombinant VSV, is immunogenic in a vertebrate host, is isolated for insertion into VSV (−) DNA according to the present invention. The species of Plasmodium which serve as DNA sources include, but are not limited to, the human malaria parasites P. falciparum, P. malariae, P. ovale, P. vivax, and the animal malaria parasites P. berghei, P. yoelii, P. knowlesi, and P. cynomolgi. In yet another embodiment, the antigen comprises a peptide of the α-subunit of Cholera toxin.

Viruses expressing epitopes that are expressed by synergistically attenuated VSV (wherein the foreign RNA directs the production of an antigen of the virus or a derivative thereof comprising an epitope thereof) include, but are not limited to, those listed in Table 2, which lists such viruses by family for purposes of convenience and not limitation.

In specific embodiments, the antigen encoded by the foreign sequences that is expressed upon infection of a host by the attenuated VSV, displays the antigenicity or immunogenicity of an influenza virus hemagglutinin; human respiratory syncytial virus G glycoprotein (G); measles virus hemagglutinin or herpes simplex virus type-2 glycoprotein gD.

TABLE 1
Parasites and Bacteria Expressing Epitopes That can be
expressed by VSV

| PARASITES | BACTERIA |
|---|---|
| plasmodium spp. | Vibrio cholerae |
| Eimeria spp. | Streptococcus pneumoniae |
| Nematodes | Streptococcus agalactiae |
| Schistosoma | Streptococcus pyogenes |
| Leishmania | Neisseria meningitidis |
| | Neisseria gonorrhoeae |
| | Corynebacterium diphtheriae |
| | Staphylococcus aureus |
| | Staphylococcus epidermidis |
| | Clostridium tetani |
| | Bordetella pertussis |
| | Haemophilus spp. (e.g., |
| | influenzae) |
| | Chlamydia spp. |
| | Enterotoxigenic Escherichia coli |
| | Helicobacter pylori |
| | mycobacteria |

TABLE 2
Viruses Expressing Epitopes that can be expressed by VSV

I. Picornaviridae
Enteroviruses
Poliovirus
Coxsackievirus
Echovirus
Rhinoviruses
Hepatitis A Virus
II. Caliciviridae
Norwalk group of viruses
III. Togaviridae and Flaviviridae
Togaviruses (e.g., Dengue virus)
Alphaviruses
Flaviviruses (e.g., Hepatitis C virus)
Rubella virus
IV. Coronaviridae
Coronaviruses
V. Rhabdoviridae
Rabies virus
VI. Filoviridae
Marburg viruses
Ebola viruses
VII. Paramyxoviridae
Parainfluenza virus
Mumps virus
Measles virus
Respiratory syncytial virus
Metapneumovirus
VIII. Orthomyxoviridae
Orthomyxoviruses (e.g., Influenza virus)
IX. Bunyaviridae
Bunyaviruses
X. Arenaviridae
Arenaviruses
XI. Reoviridae
Reoviruses
Rotaviruses
Orbiviruses
XII. Retroviridae
Human T Cell Leukemia Virus type I
Human T Cell Leukemia Virus type II
Human Immunodeficiency Viruses (e.g., type
I and type II
Simian Immunodeficiency Virus
Lentiviruses
XIII. Papovaviridae
Polyomaviruses
Papillomaviruses
XIV. Parvoviridae
Parvoviruses
XV. Herpesviridae
Herpes Simplex Viruses
Epstein-Barr virus
Cytomegalovirus
Varicella-Zoster virus
Human Herpesvirus-6
human herpesvirus-7
Cercopithecine Herpes Virus 1 (B virus)
XVI. Poxviridae
Poxviruses
XVIII. Hepadnaviridae
Hepatitis B virus
XIX. Adenoviridae

Other antigens that are expressed by attenuated VSV include, but are not limited to, those displaying the antigenicity or immunogenicity of the following antigens: Poliovirus I VP1; envelope glycoproteins of HIV I; Hepatitis B surface antigen; Diphtheria toxin; streptococcus 24M epitope, SpeA, SpeB, SpeC or C5a peptidase; and gonococcal pilin.

In other embodiments, the antigen expressed by the attenuated and further genetically modified VSV displays the antigenicity or immunogenicity of pseudorabies virus g50 (gpD), pseudorabies virus II (gpB), pseudorabies virus gill (gpC), pseudorabies virus glycoprotein H, pseudorabies virus glycoprotein E, transmissible gastroenteritis glycoprotein 195, transmissible gastroenteritis matrix protein, swine rotavirus glycoprotein 38, swine parvovirus capsid protein, Serpulina hydrodysenteriae protective antigen, Bovine Viral Diarrhea glycoprotein 55, Newcastle Disease Virus hemagglutinin-neuraminidase, swine flu hemagglutinin, or swine flu neuraminidase.

In certain embodiments, an antigen expressed by the attenuated and further genetically modified VSV displays the antigenicity or immunogenicity of an antigen derived from a canine or feline pathogen, including, but not limited to, feline leukemia virus, canine distemper virus, canine adenovirus, canine parvovirus and the like.

In certain other embodiments, the antigen expressed by the attenuated and further genetically modified VSV displays the antigenicity or immunogenicity of an antigen derived from Serpulina hyodysenteriae, Foot and Mouth Disease Virus, Hog Cholera Virus, swine influenza virus, African Swine Fever Virus, Mycoplasma hyopneumoniae, infectious bovine rhinotracheitis virus (e.g., infectious bovine rhinotracheitis virus glycoprotein E or glycoprotein G), or infectious laryngotracheitis virus (e.g., infectious laryngotracheitis virus glycoprotein G or glycoprotein 1).

In another embodiment, the antigen displays the antigenicity or immunogenicity of a glycoprotein of La Crosse Virus, Neonatal Calf Diarrhea Virus, Venezuelan Equine Encephalomyelitis Virus, Punta Toro Virus, Murine Leukemia Virus or Mouse Mammary Tumor Virus.

In other embodiments, the antigen displays the antigenicity or immunogenicity of an antigen of a human pathogen, including but not limited to human herpesvirus, herpes simplex virus-1, herpes simplex virus-2, human cytomegalovirus, Epstein-Barr virus, Varicella-Zoster virus, human herpesvirus-6, human herpesvirus-7, human influenza virus, human immunodeficiency virus (type 1 and/or type 2), rabies virus, measles virus, hepatitis B virus, hepatitis C virus, Plasmodium falciparum, and Bordetella pertussis.

Potentially useful antigens or derivatives thereof for use as antigens expressed by attenuated VSV are identified by various criteria, such as the antigen's involvement in neutralization of a pathogen's infectivity, type or group specificity, recognition by patients' antisera or immune cells, and/or the demonstration of protective effects of antisera or immune cells specific for the antigen.

In another embodiment, foreign RNA of the attenuated VSV directs the production of an antigen comprising an epitope, which when the attenuated VSV is introduced into a desired host, induces an immune response that protects against a condition or disorder caused by an entity containing the epitope. For example, the antigen can be a tumor specific antigen or tumor-associated antigen, for induction of a protective immune response against a tumor (e.g., a malignant tumor). Such tumor-specific or tumor-associated antigens include, but are not limited to, KS ¼ pan-carcinoma antigen; ovarian carcinoma antigen (CA125); prostatic acid phosphate; prostate specific antigen; melanoma-associated antigen p97; melanoma antigen gp75; high molecular weight melanoma antigen and prostate specific membrane antigen.

The foreign DNA encoding the antigen, that is inserted into a non-essential site of the attenuated VSV DNA, optionally further comprises a foreign DNA sequence encoding a cytokine capable of being expressed and stimulating an immune response in a host infected by the attenuated VSV. For example, such cytokines include but are not limited to interleukins 1α, 1β, 2, 4, 5, 6, 7, 8, 10, 12, 13, 14, 15, 16, 17 and 18, interferon-α, interferon-β, interferon-γ, granulocyte colony stimulating factor, granulocyte macrophage colony stimulating factor and the tumor necrosis factors α and β.

### Immunogenic and Pharmaceutical Compositions

In certain embodiments, the invention is directed to an immunogenic composition comprising an immunogenic dose of a genetically modified VSV vector comprising at least two different classes of mutations in its genome and at least one foreign RNA sequence inserted into or replacing a region of the VSV genome non-essential for replication, wherein the two mutations synergistically attenuate VSV pathogenicity. The genetically modified VSV may be further adapted for growth in cell culture by passaging the virus for about 5 to 15 passages at a low MOI as described herein and this further genetically modified and attenuated VSV may be used to prepare the immunogenic compositions.

The synergistically attenuated and genetically modified VSV vectors of the invention are formulated for administration to a mammalian subject (e.g., a human). Such compositions typically comprise the VSV vector and a pharmaceutically acceptable carrier. As used hereinafter the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the VSV vector, such media are used in the immunogenic compositions of the invention. Supplementary active compounds may also be incorporated into the compositions.

Thus, a VSV immunogenic composition of the invention is formulated to be compatible with its intended route of administration. Examples of routes of administration include parenteral (e.g., intravenous, intradermal, subcutaneous, intramuscular, intraperitoneal) and mucosal (e.g., oral, rectal, intranasal, buccal, vaginal, respiratory). Solutions or suspensions used for parenteral, intradermal, or subcutaneous application include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH is adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier is a solvent or dispersion medium containing, for example, water, ethanol, polyol (e.g., glycerol, propylene glycol, and liquid polyethylene glycol, and the like), and suitable mixtures thereof. The proper fluidity is maintained, for example, by the use of a coating such as lecithin, by the maintenance of the required particle size in the case of dispersion and by the use of surfactants. Prevention of the action of microorganisms is achieved by various antibacterial and antifungal agents, for example, parabens, chlorobutanol, phenol, ascorbic acid, and the like. In many cases, it is preferable to include isotonic agents, for example, sugars, polyalcohols such as mannitol, sorbitol, sodium chloride in the composition. Prolonged absorption of the injectable compositions is brought about by including in the composition an agent which delays absorption, for example, aluminum monostearate and gelatin.

Sterile injectable solutions are prepared by incorporating the VSV vector in the required amount (or dose) in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

For administration by inhalation, the compounds are delivered in the form of an aerosol spray from pressured container or dispenser which contains a suitable propellant (e.g., a gas such as carbon dioxide, or a nebulizer). Systemic administration can also be by mucosal or transdermal means. For mucosal or transdermal administration, penetrants appropriate to the barrier to be permeated are used in the formulation. Such penetrants are generally known in the art, and include, for example, for mucosal administration, detergents, bile salts, and fusidic acid derivatives. Mucosal administration is accomplished through the use of nasal sprays or suppositories. The compounds are also prepared in the form of suppositories (e.g., with conventional suppository bases such as cocoa butter and other glycerides) or retention enemas for rectal delivery.

In certain embodiments, it is advantageous to formulate oral or parenteral compositions in dosage unit form for ease of administration and uniformity of dosage. Dosage unit form as used hereinafter refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

All patents and publications cited herein are hereby incorporated by reference.

The production of VSV vectored immunogenic compositions generally includes infecting a suitable cell culture (host) with recombinant VSV, growing VSV in cell culture, harvesting the cell culture fluid at the appropriate time and purifying the VSV from the cell culture fluid. The use of VSV vectors, and immunogenic compositions thereof, in clinical applications will require VSV samples (or doses) of appropriate purity in order to comply with safety regulations of the various drug safety authorities around the world (e.g., the Food and Drug Administration (FDA), the European Medicines Agency (EMEA), the Canadian Health Products and Food Branch (HPFB), etc.).

However, it is typically difficult to separate VSV from the cell culture contaminants (e.g., cell culture impurity proteins and DNA) and obtain VSV of appropriate purity and yield using the currently available VSV purification processes (e.g., purification via sucrose gradient centrifugation). For example, using the currently available purification processes, there is typically an inverse relationship between the purity and recovery (percent yield) of VSV samples, thereby making it difficult to manufacture sufficient quantities of purified VSV. Additionally, in today's bioreactor-based processes, increased cell concentrations and longer culture times result in higher VSV titers, with concomitant increases in cell debris and concentrations of organic constituents in the bioreactor fluid, further complicating VSV purification processes.

Sucrose gradient ultracentrifugation has been the standard method for virus purification (including VSV purification) since 1964 (Yamada et al., 2003 BioTechniques, 34(5):1074-1078, 1080; Brown et al., 1967 J. Immun., 99(1):171-7; Robinson et al., 1965 Proc. Natl. Acad. Sci., USA, 54(1):137-44; Nishimura et al., 1964 Japan. J. Med. Sci. Biol., 17(6):295-305). However, as virus concentrations increase, concomitant increases in cell debris, host DNA and protein impurities also occur, which are very difficult to remove at higher concentrations via sucrose gradient ultracentrifugation. In addition, sucrose gradient ultracentrifugation is extremely costly to scale-up. Concentration and purification of VSV by polyethylene glycol (PEG) precipitation (McSharry et al., 1970 Virol., 40(3):745-6) has similar problems of high impurity levels.

Relatively high quality virus has been obtained via size exclusion chromatography (Transfiguracion et al., 2003 Human Gene Ther., 14(12):1139-1153; Vellekamp, et al., 2001 Human Gene Ther., 12(15):1923-36; Rabotti et al., 1971 Comptes Rendus des Seances de l'Academie des Sciences, Serie D: Sciences Naturelles, 272(2):343-6; Jacoli et al., 1968 Biochim. Biophys. Acta, Genl Subj., 165(2):99-302). However, due to process cost and operating difficulty, it is generally not feasible for large-scale virus production. Affinity chromatography, such as heparin (Zolotukhin et al., 1999 Gene Ther., 6(6):973-985), lectin (Kaarsnaes et al., 1983 J. Chromatog., 266:643-9; Kristiansen et al., 1976 Prot. Biol. Fluids, 23:663-5) and Matrex™ Cellufine™ sulfate (Downing et al., 1992 J. Virol. Meth., 38(2):215-228), has found some application in virus purification. Heparin and lectin are generally not preferred (or used) for cGMP virus production due to possible leaching problems, which would require additional tests prior to product release.

Affinity purification of virus using Matrex™ Cellufine™sulfate is an unresolved issue, due to efficiency of virus purification, virus quality and column regeneration. For VSV purification, very large affinity columns are needed (e.g., 0.2 L Matrex™ Cellufine™ sulfate resin per liter of cell culture; Wyeth Vaccine unpublished results). Low virus yield was observed when purified via ion exchange chromatography, either alone, or in combination with other types of traditional chromatographic techniques used in virus purification (International Patent Publication No. WO2006/011580; Specht et al., 2004 Biotech. Bioeng., 88(4):465-173; Yamada et al., 2003, cited above; Vellekamp et al., 2001 cited above; Zolotukhin et al., 1999, cited above; (International Patent Publication No. WO1997/06243; Kaarsnaes et al., 1983, cited above).

### EXAMPLES

The following examples demonstrate certain aspects of the present invention. However, it is to be understood that these examples are for illustration only and do not purport to be wholly definitive as to conditions and scope of this invention. It should be appreciated that when typical reaction conditions (e.g., temperature, reaction times, etc.) have been given, the conditions both above and below the specified ranges can also be used, though generally less conveniently. All parts and percents referred to herein are on a weight basis and all temperatures are expressed in degrees centigrade unless otherwise specified.

### Example 1

### Virus Passage Study with the VSV Indiana (IN) and VSV New Jersey (NJ) Serotype

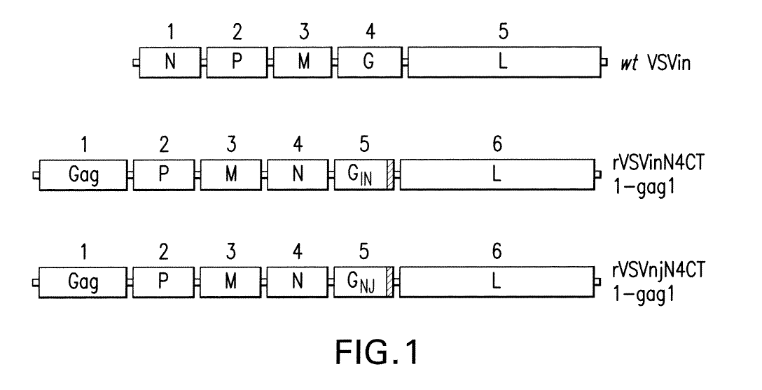

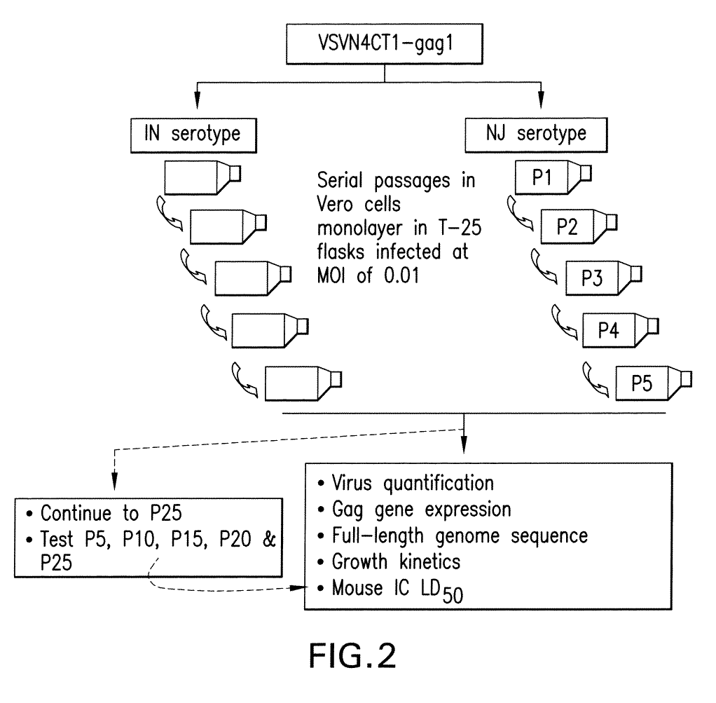

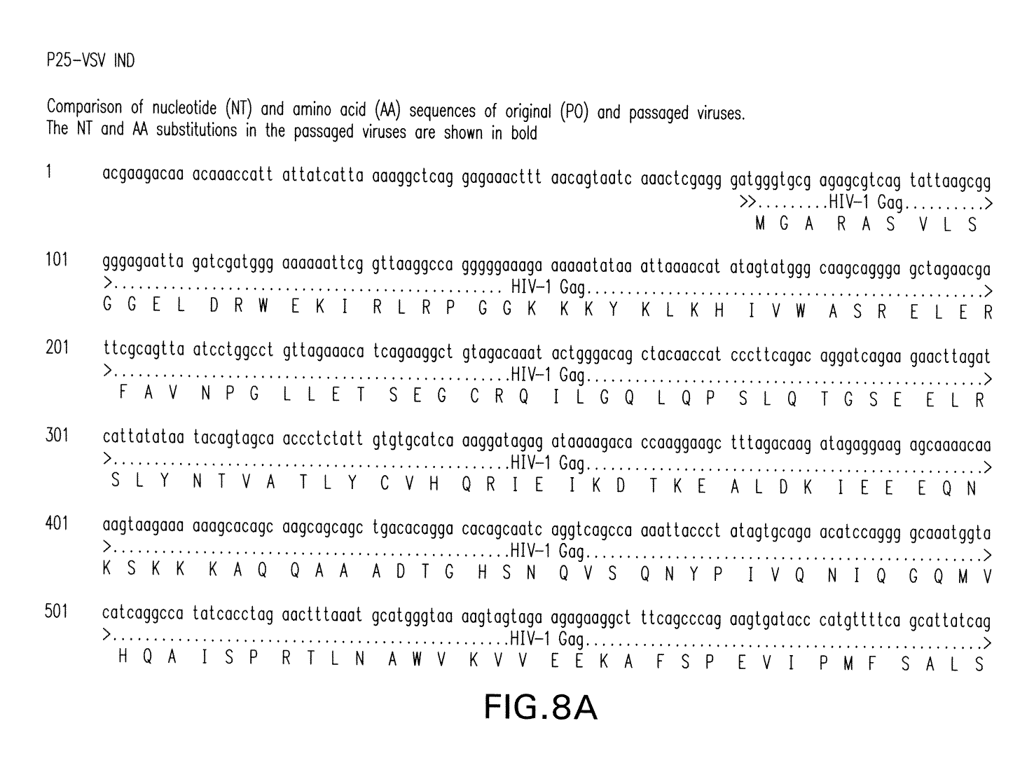

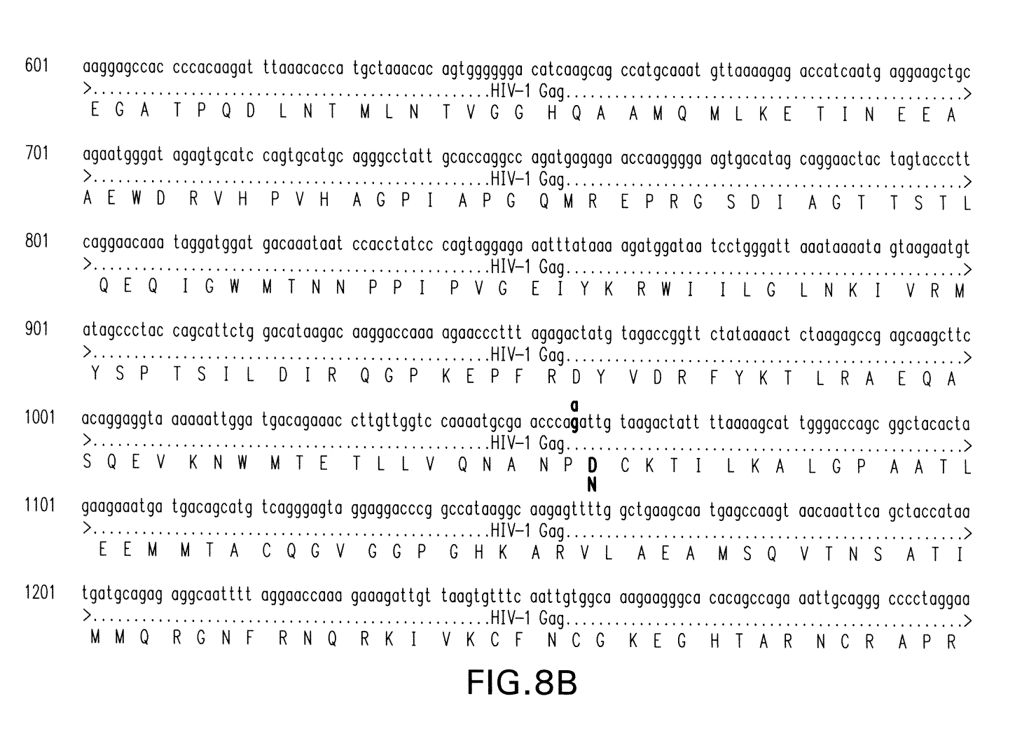

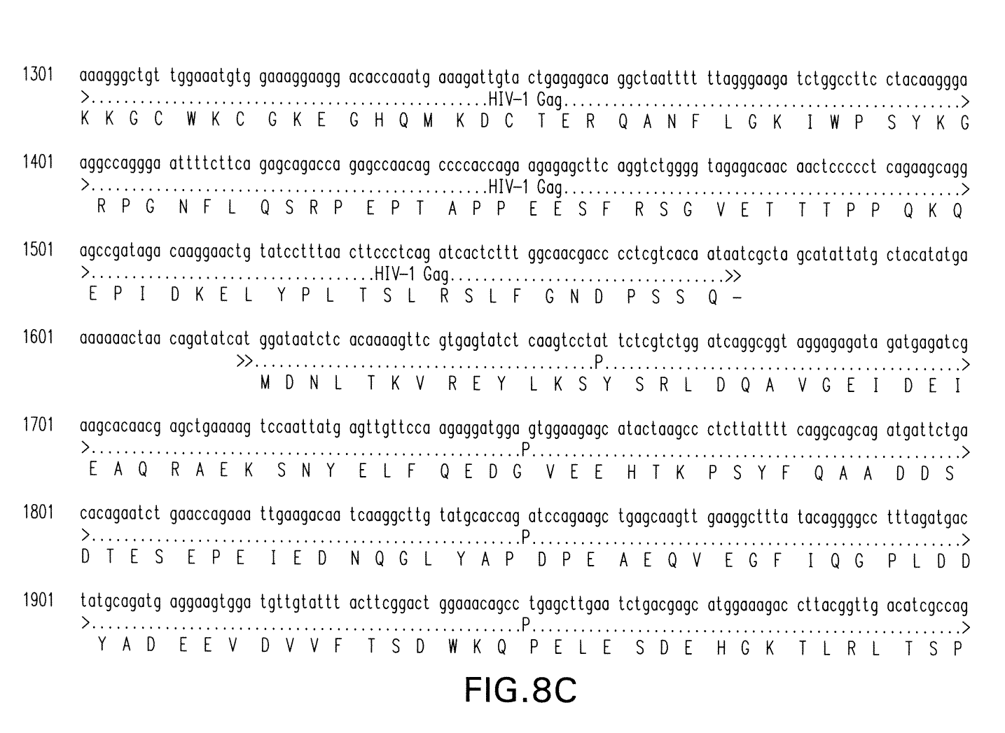

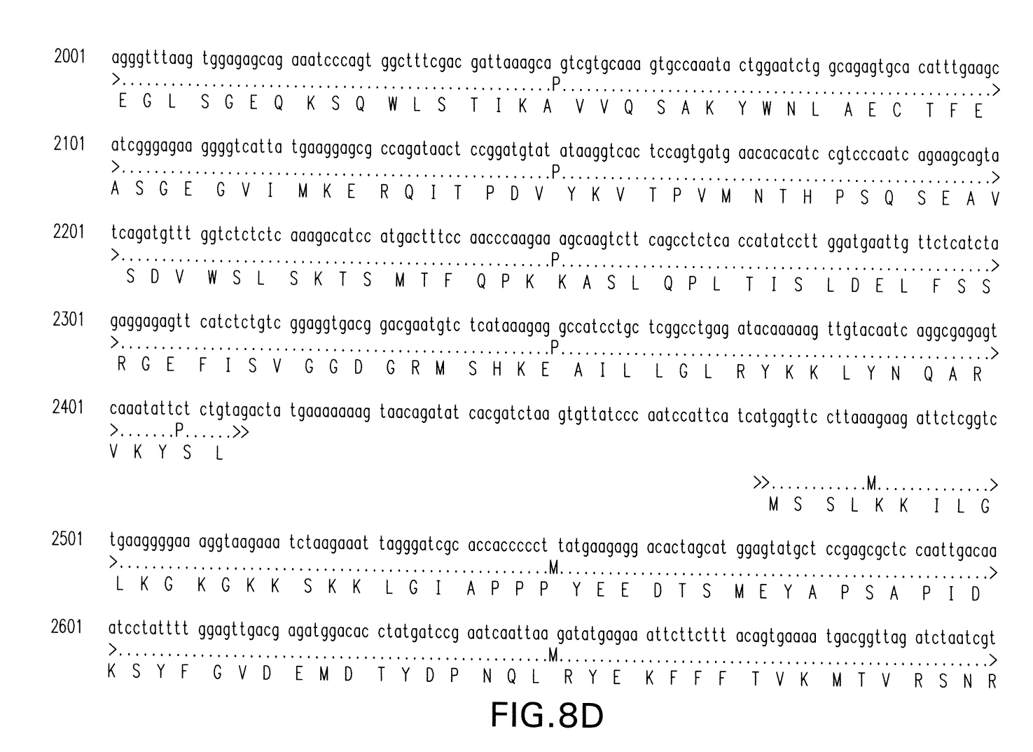

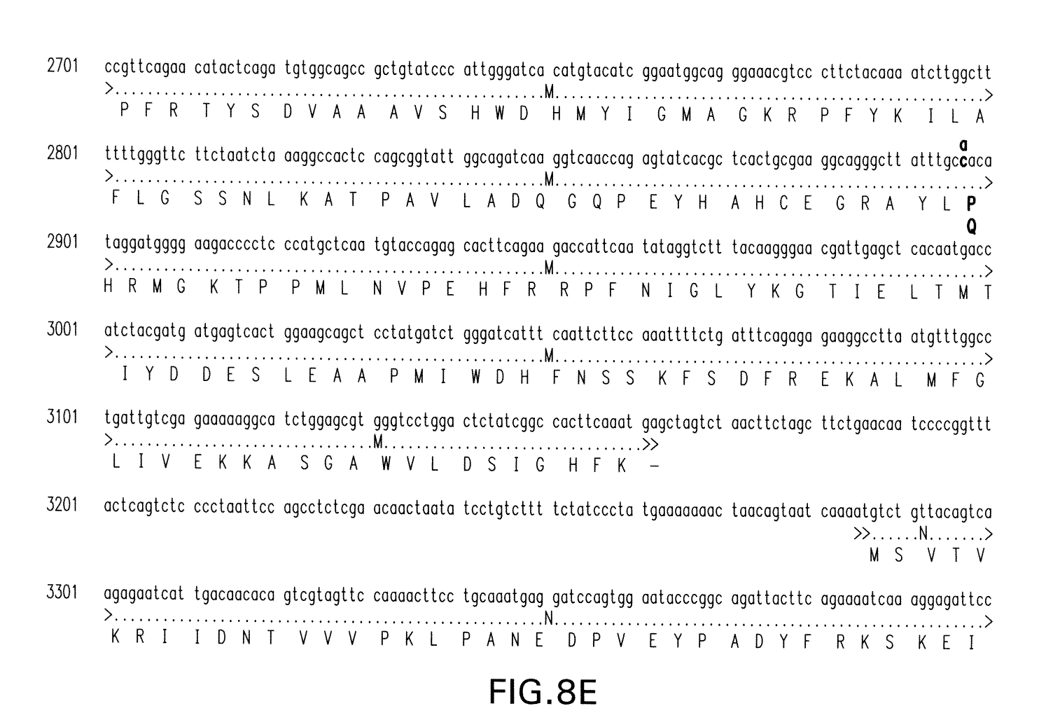

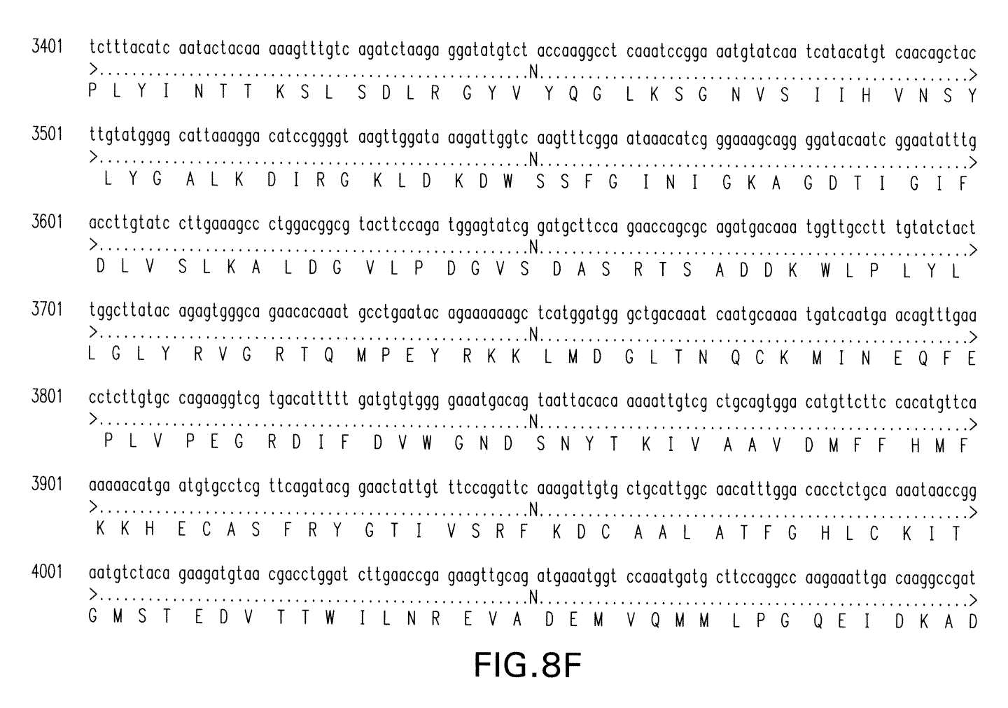

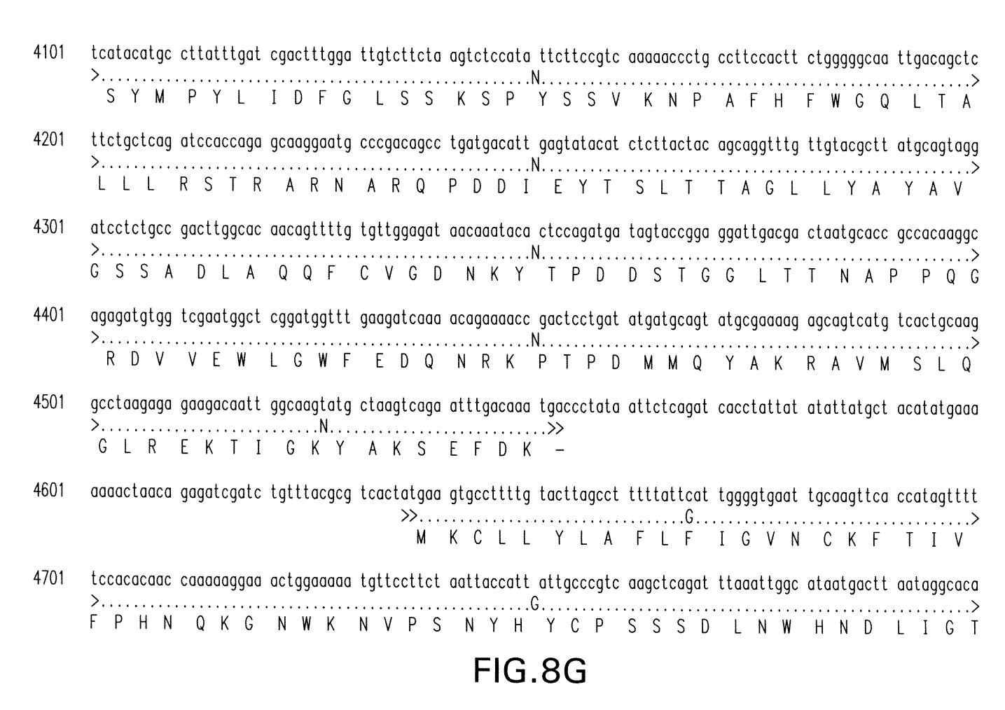

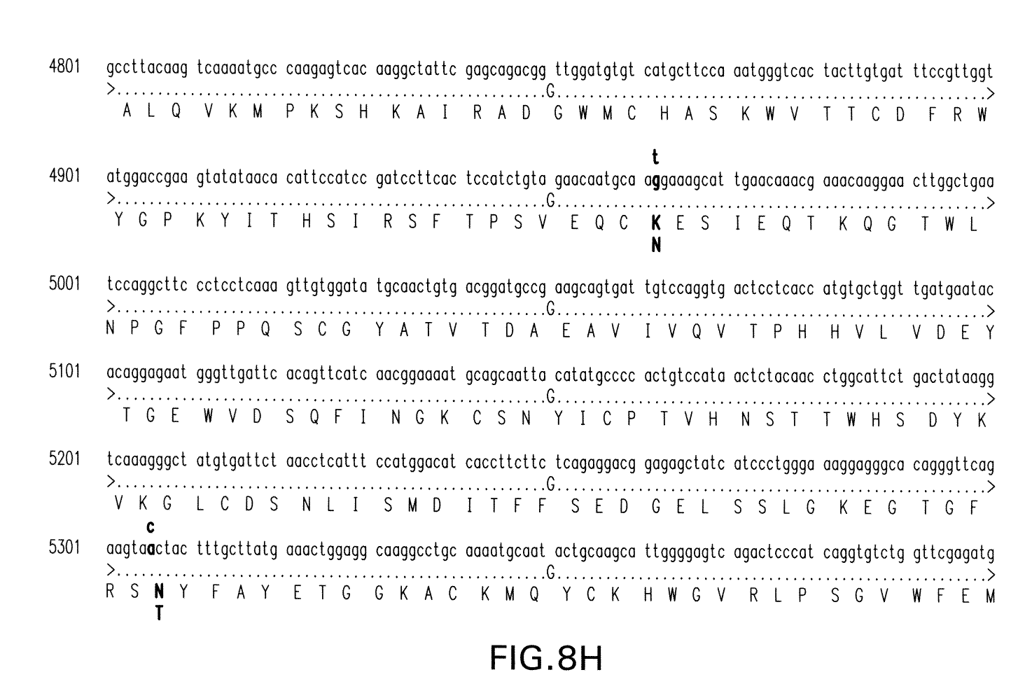

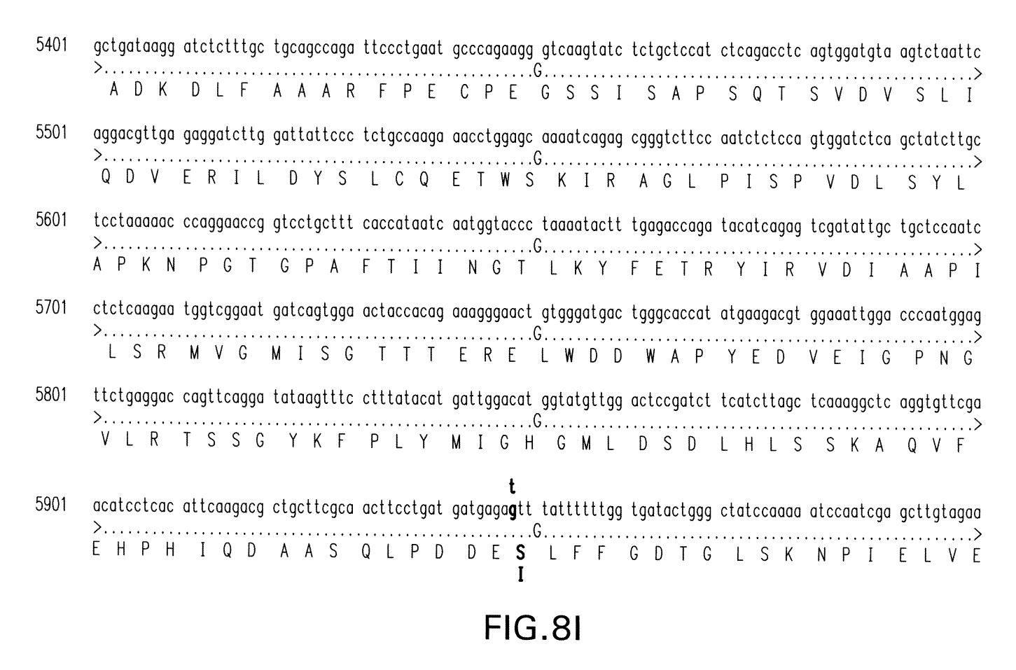

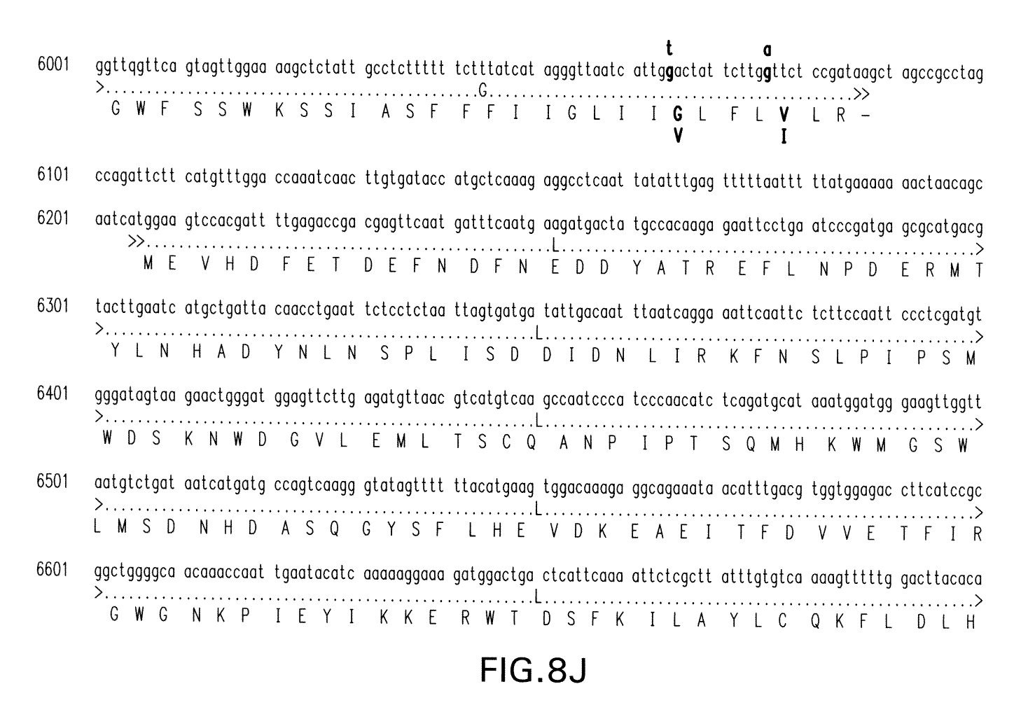

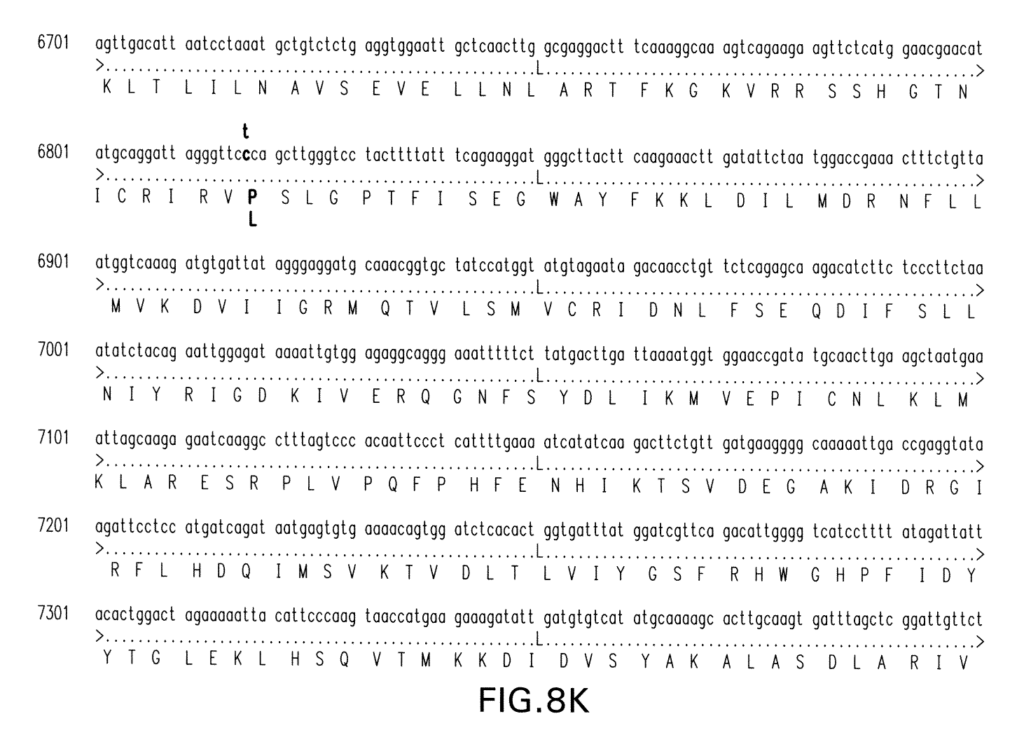

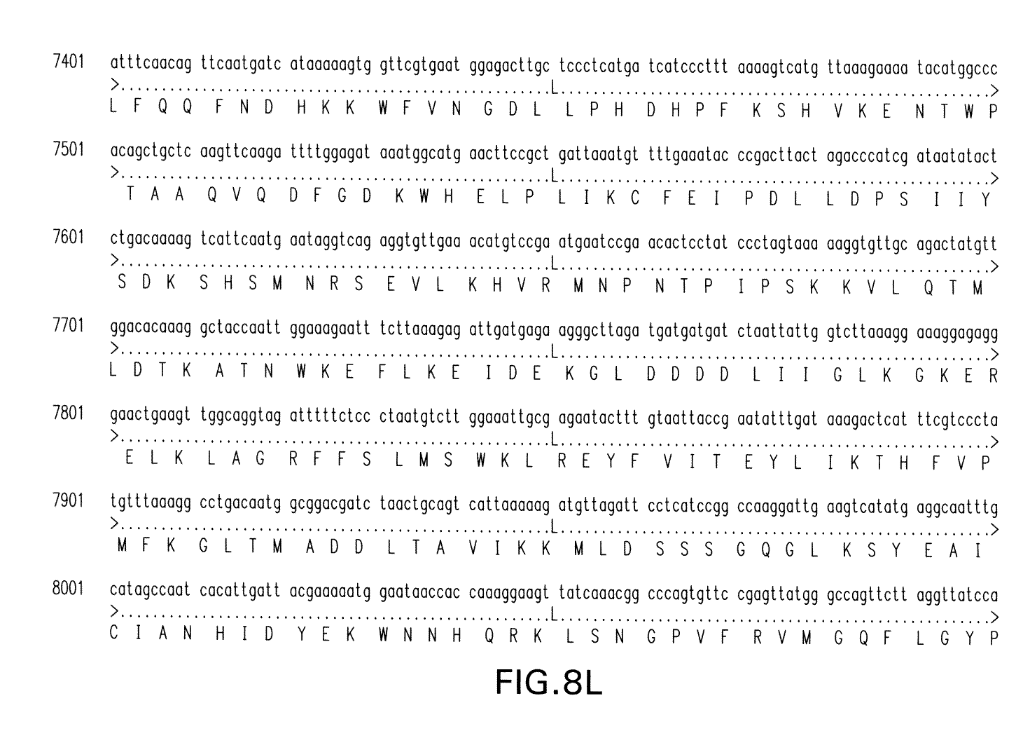

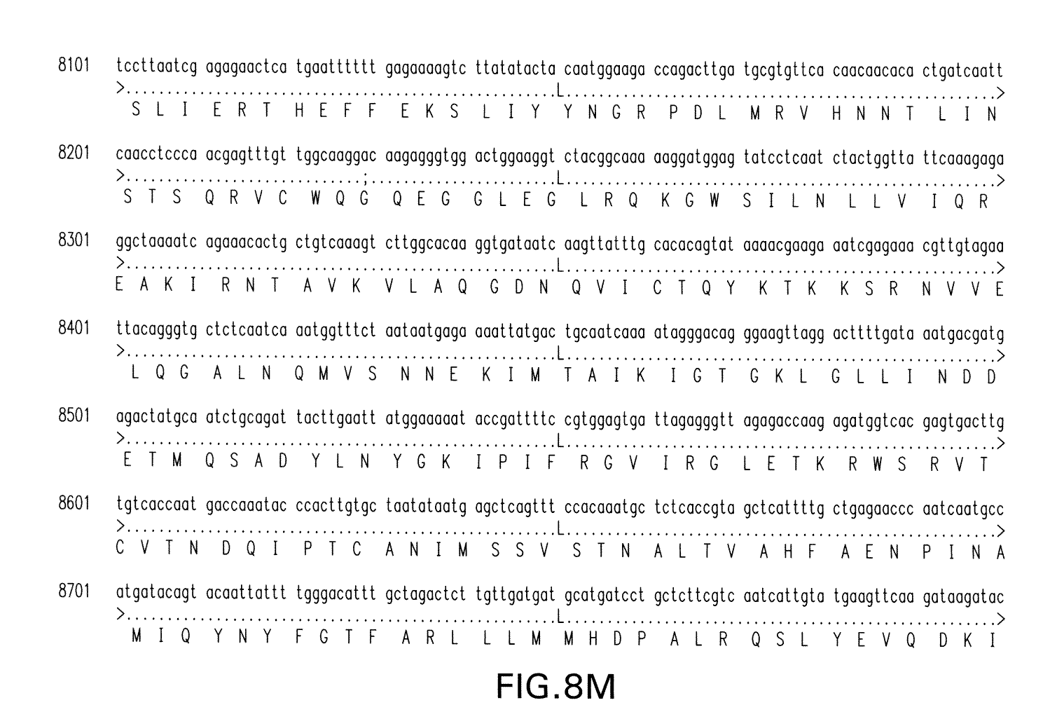

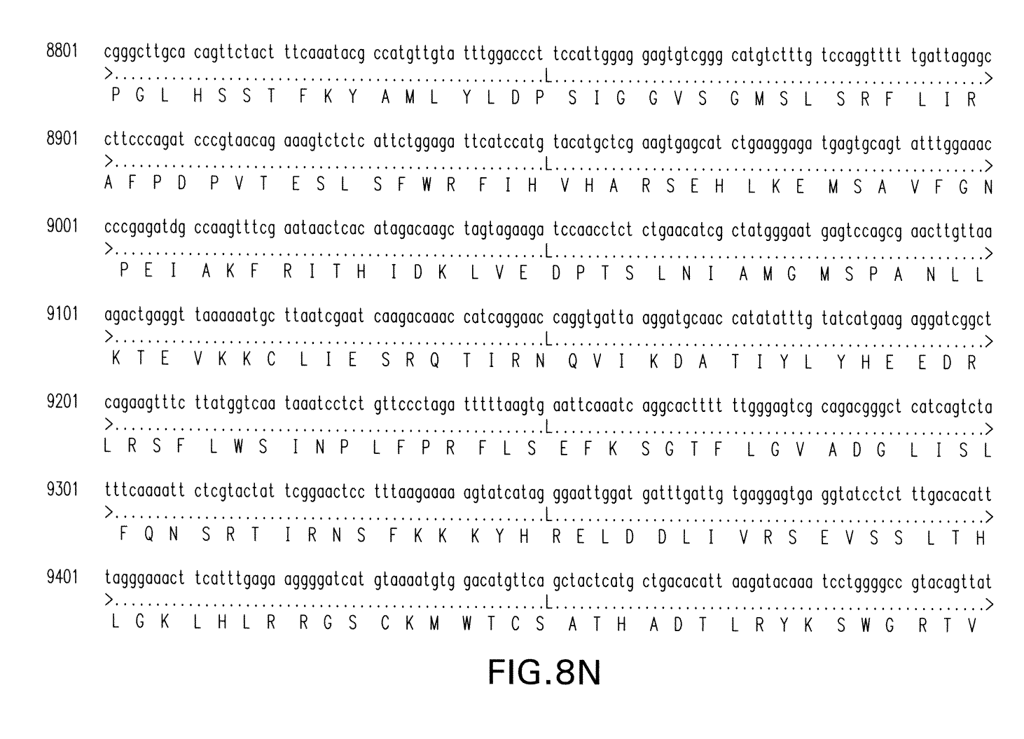

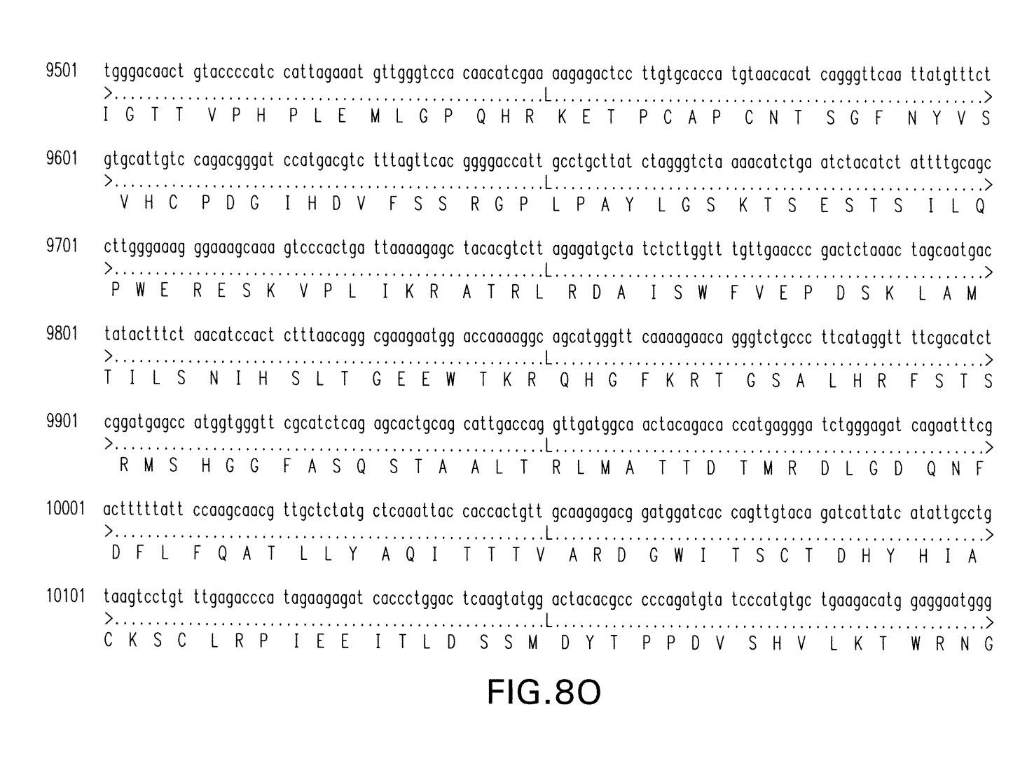

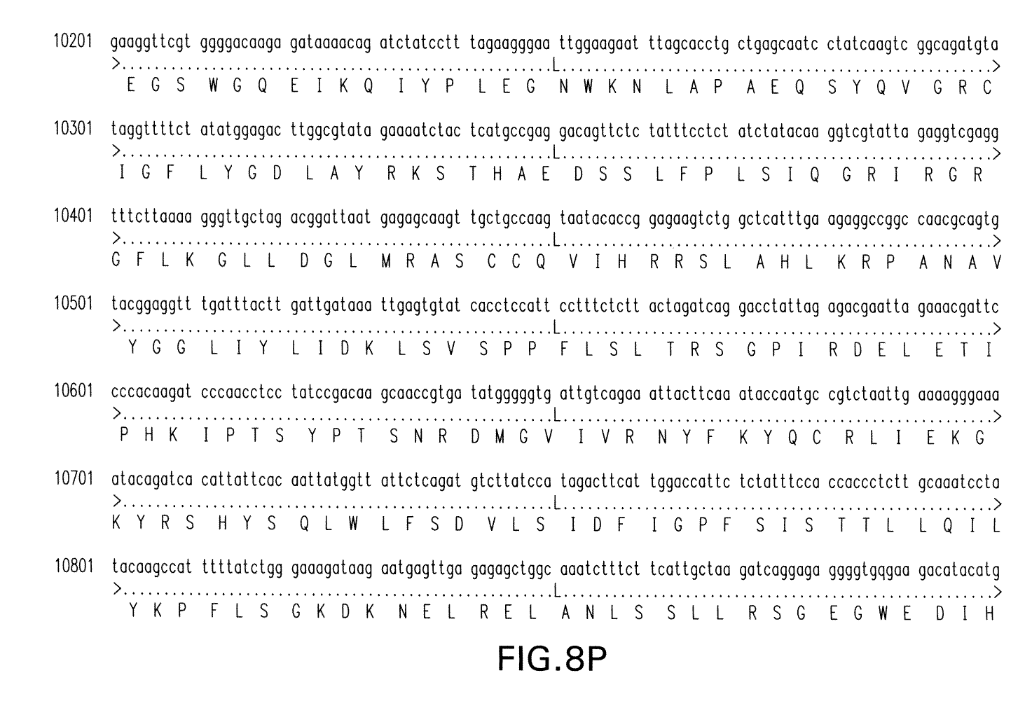

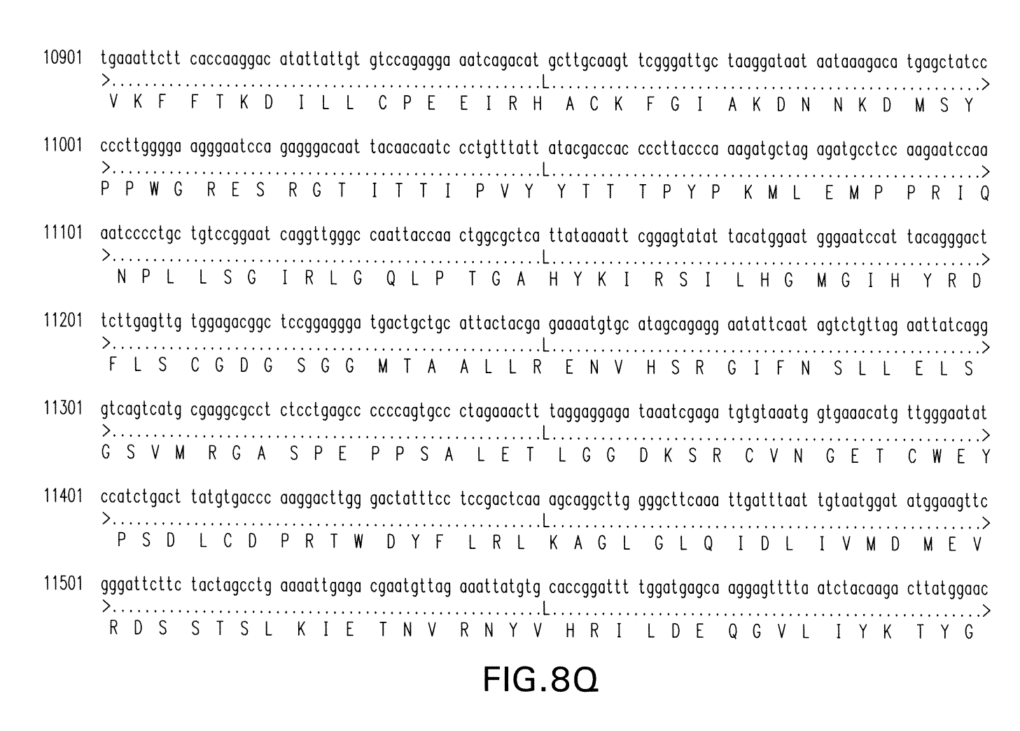

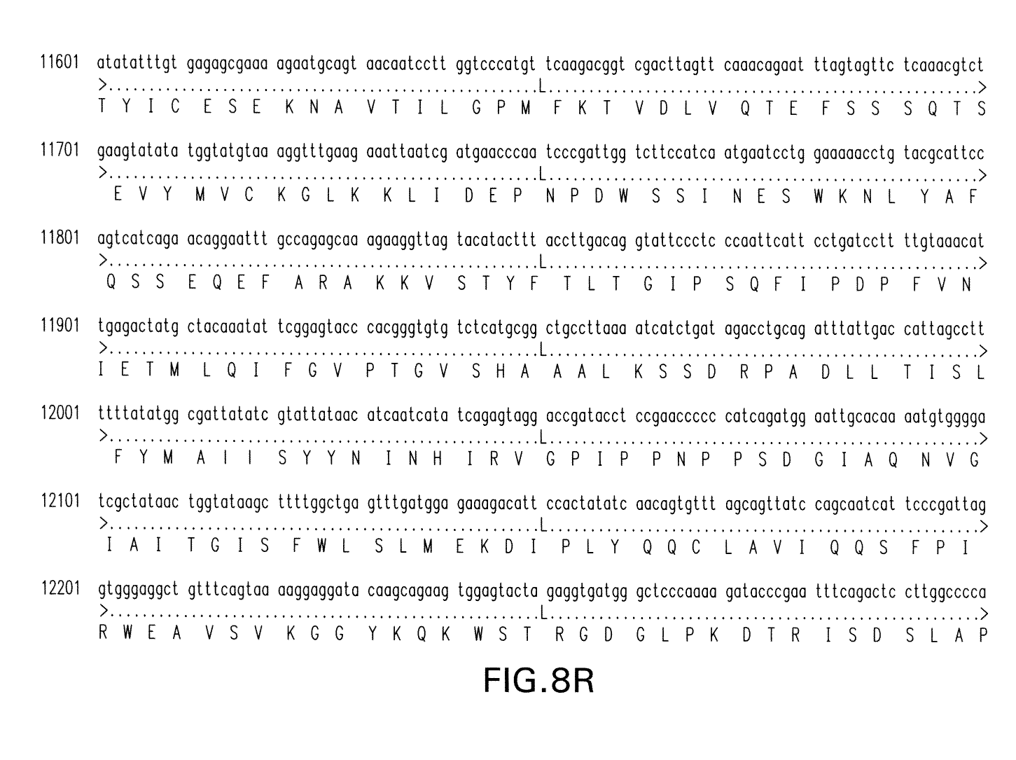

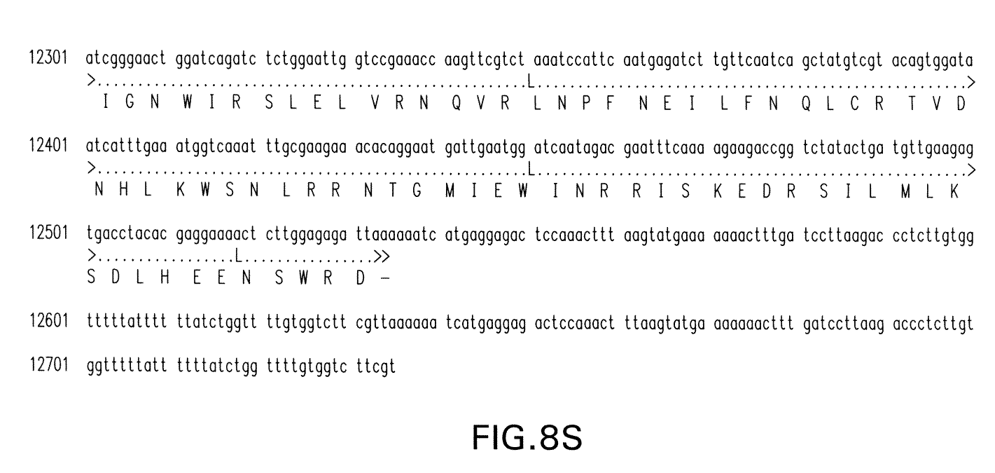

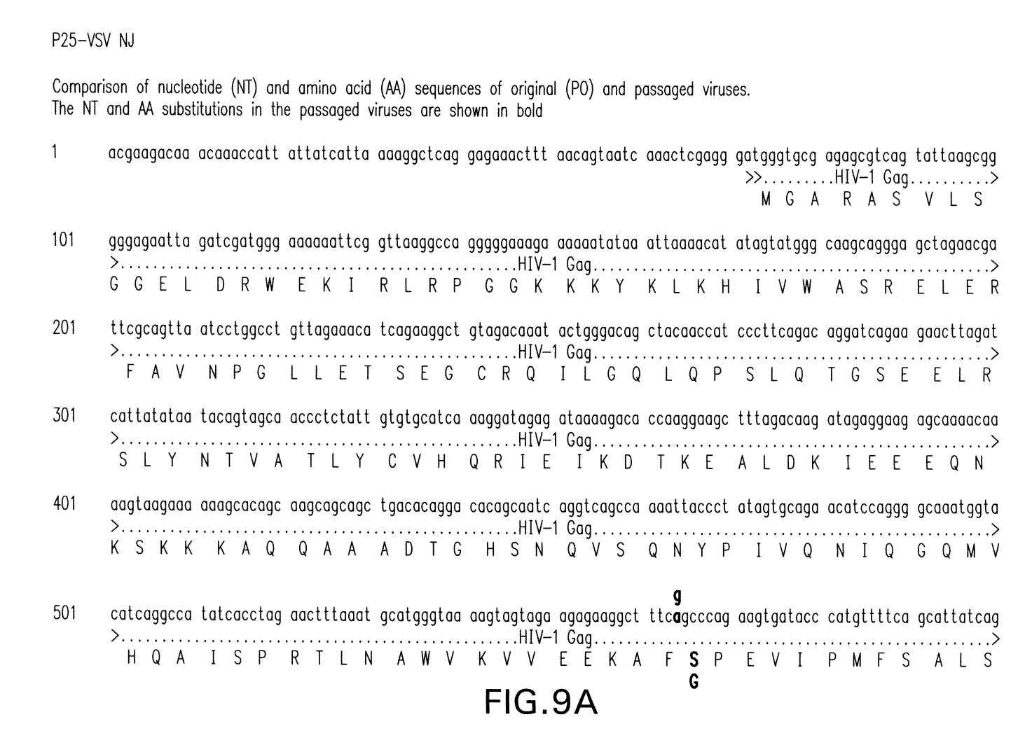

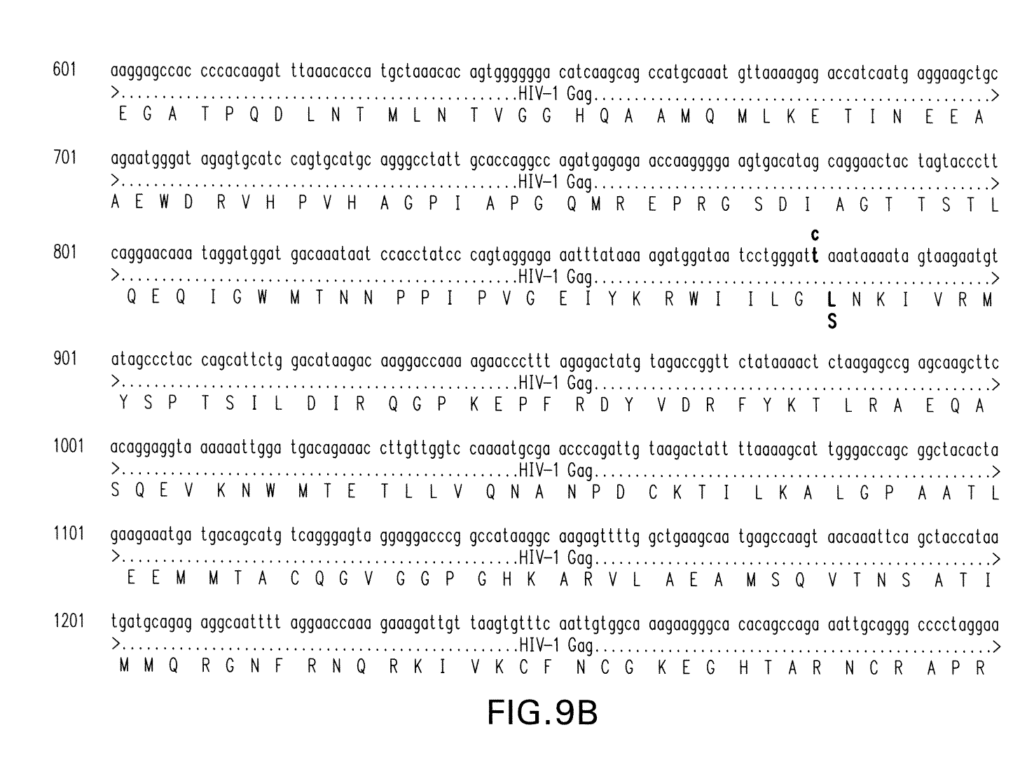

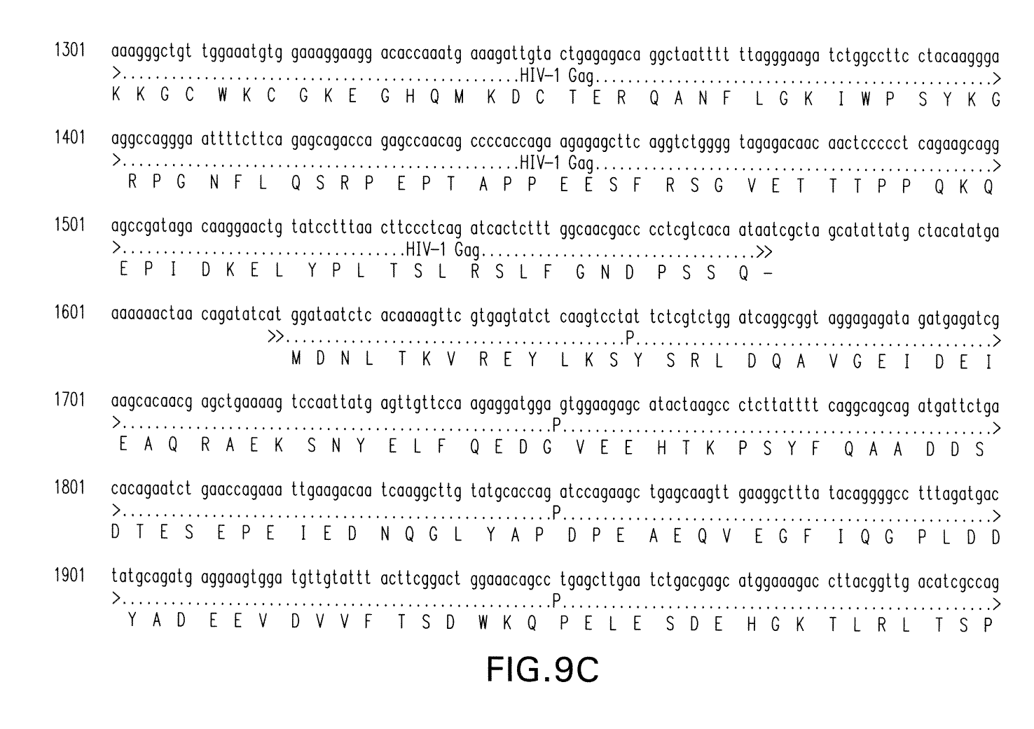

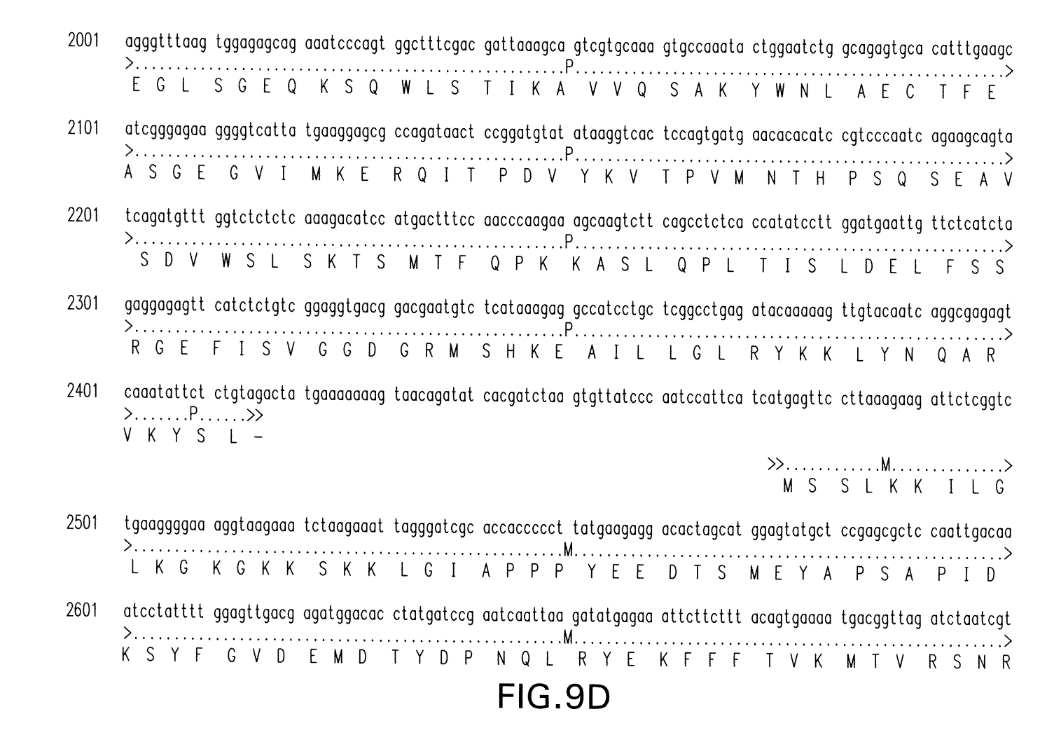

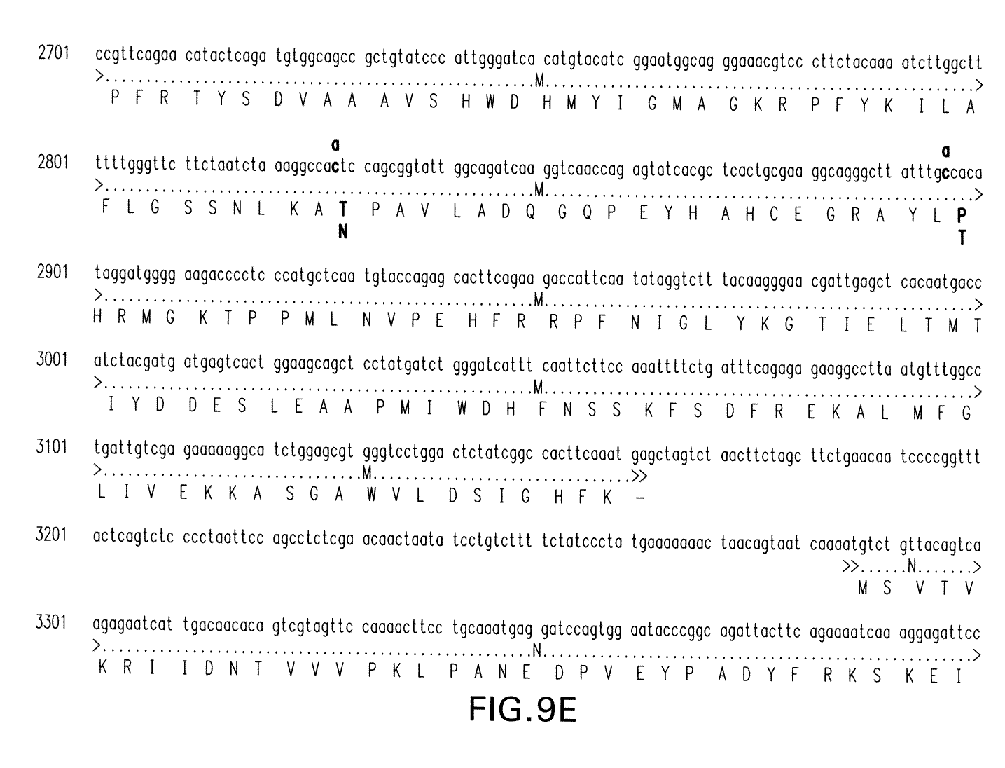

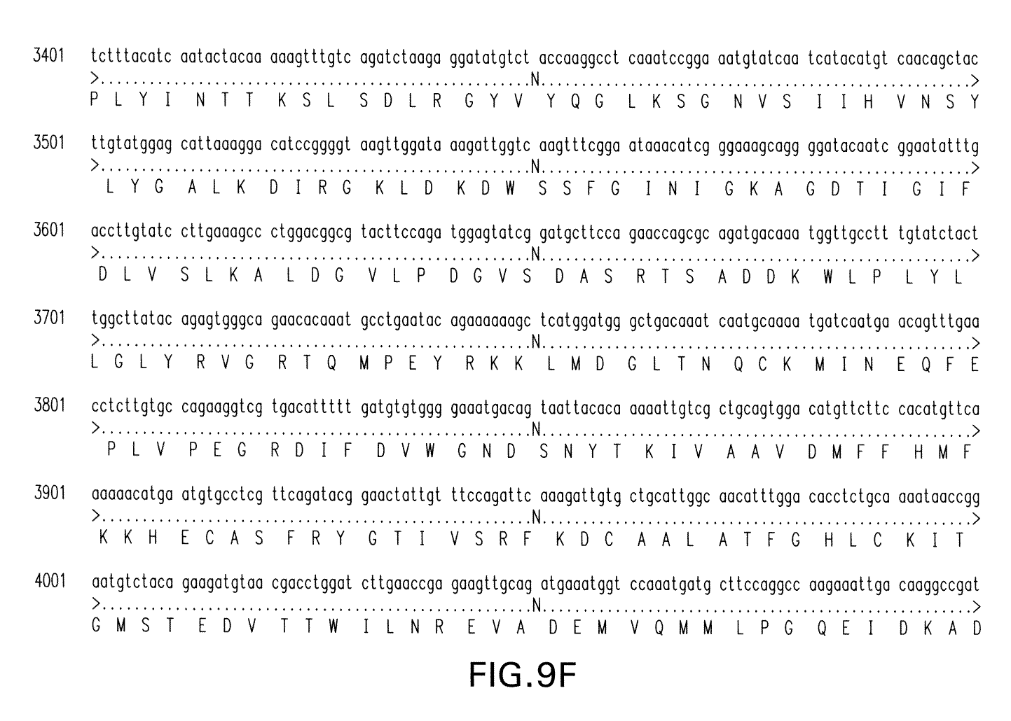

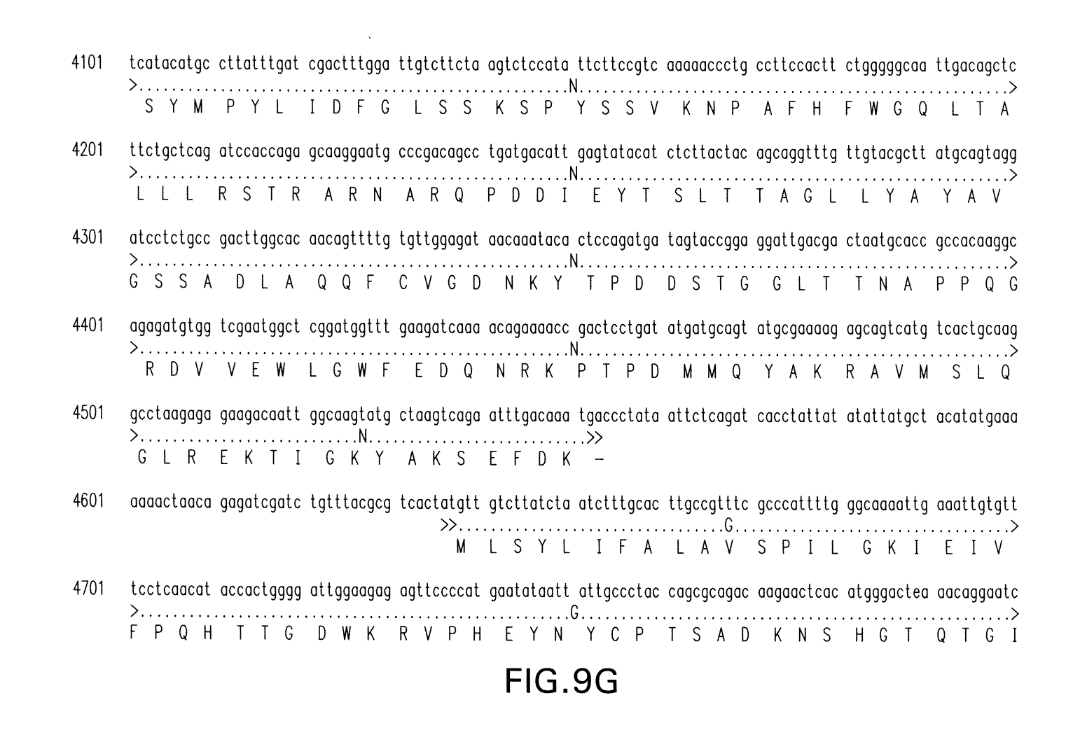

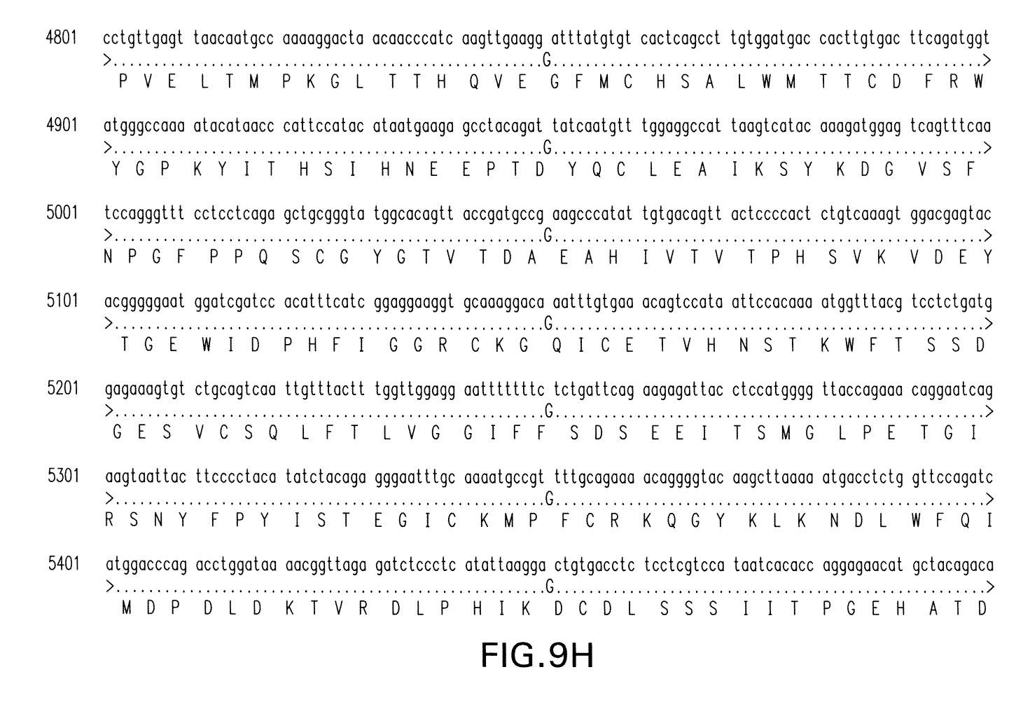

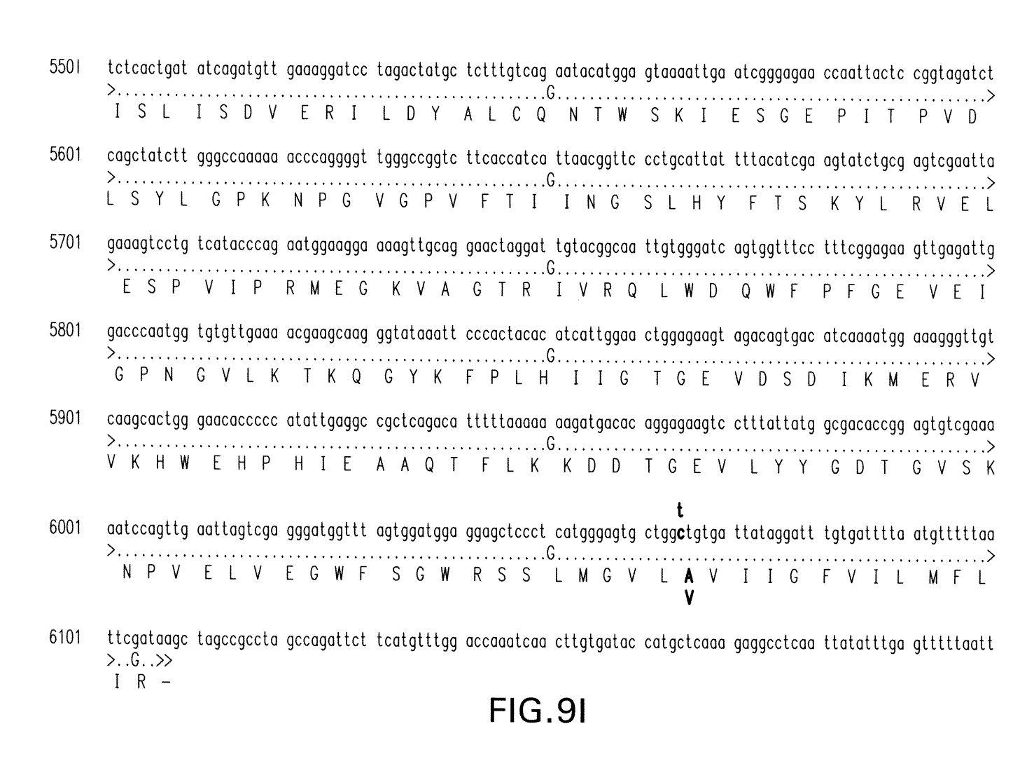

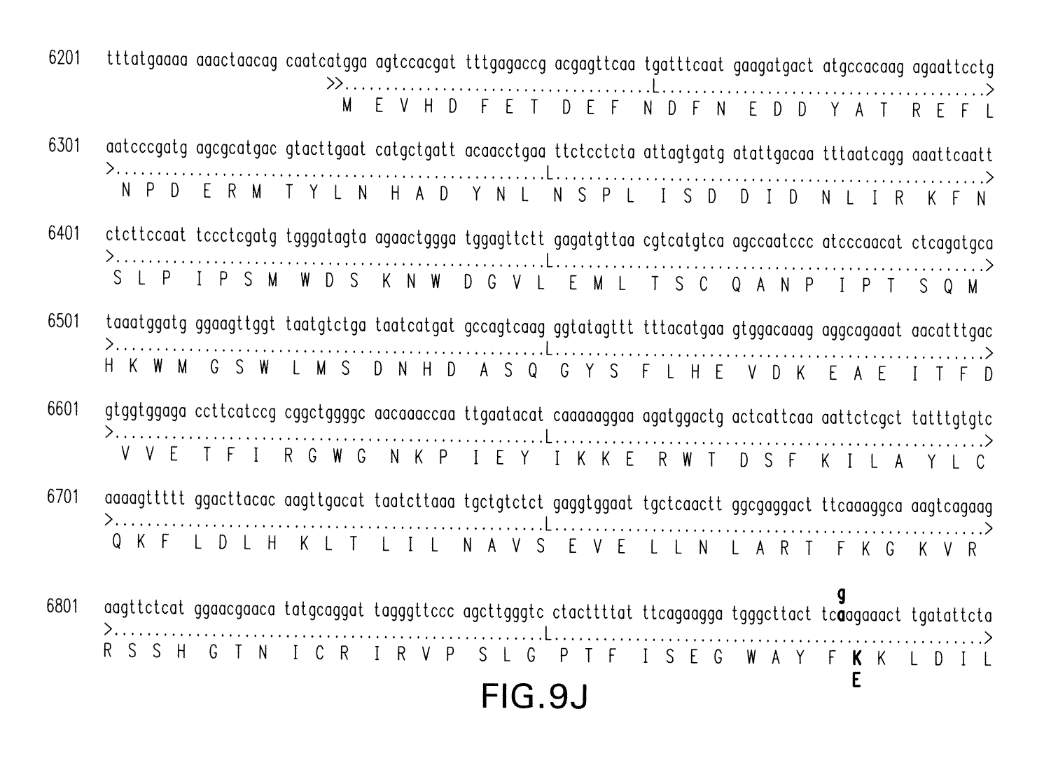

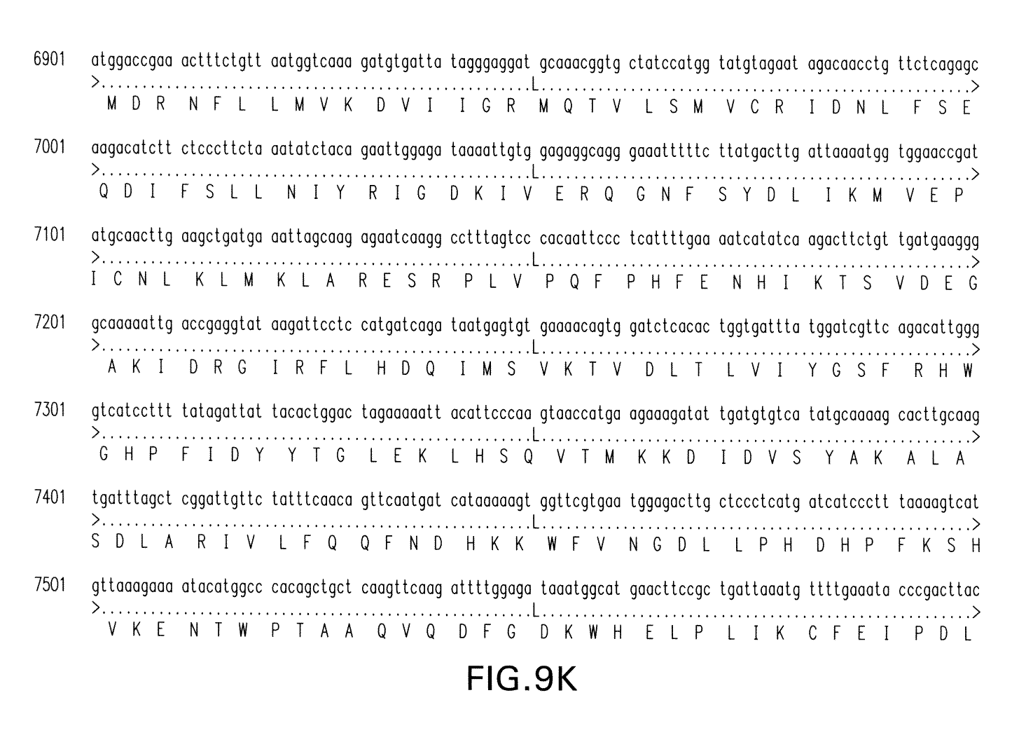

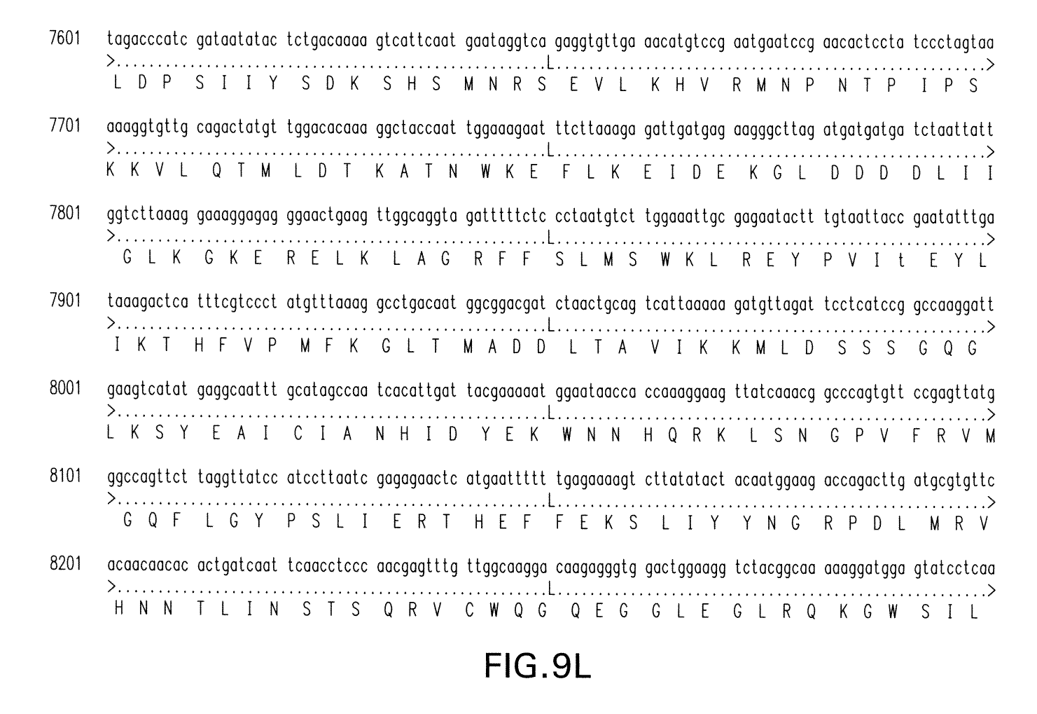

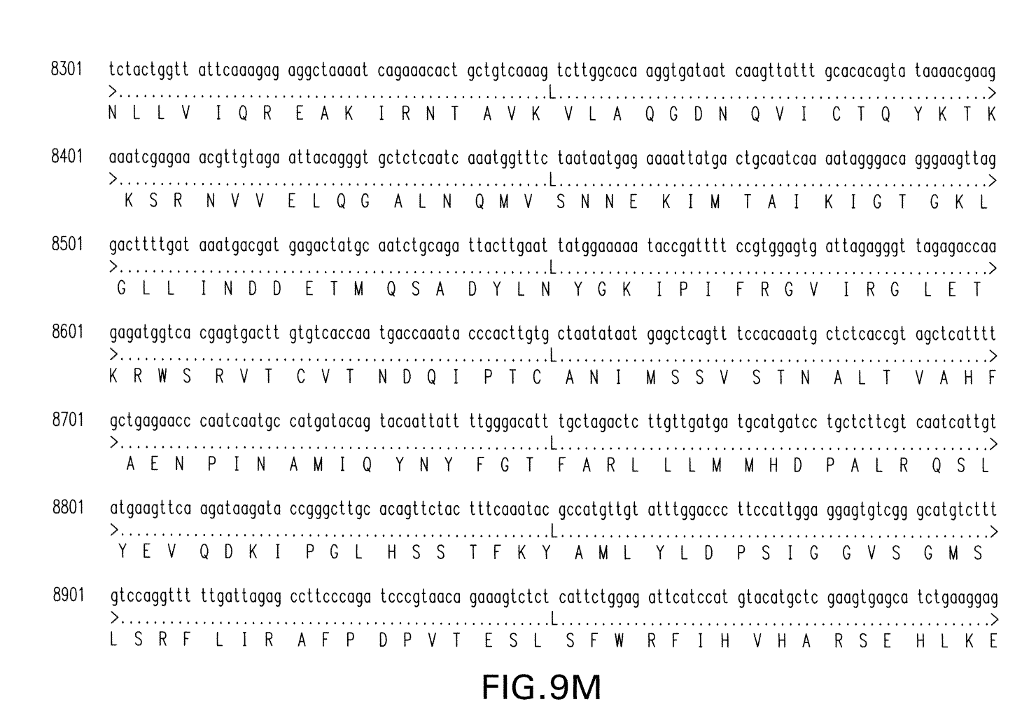

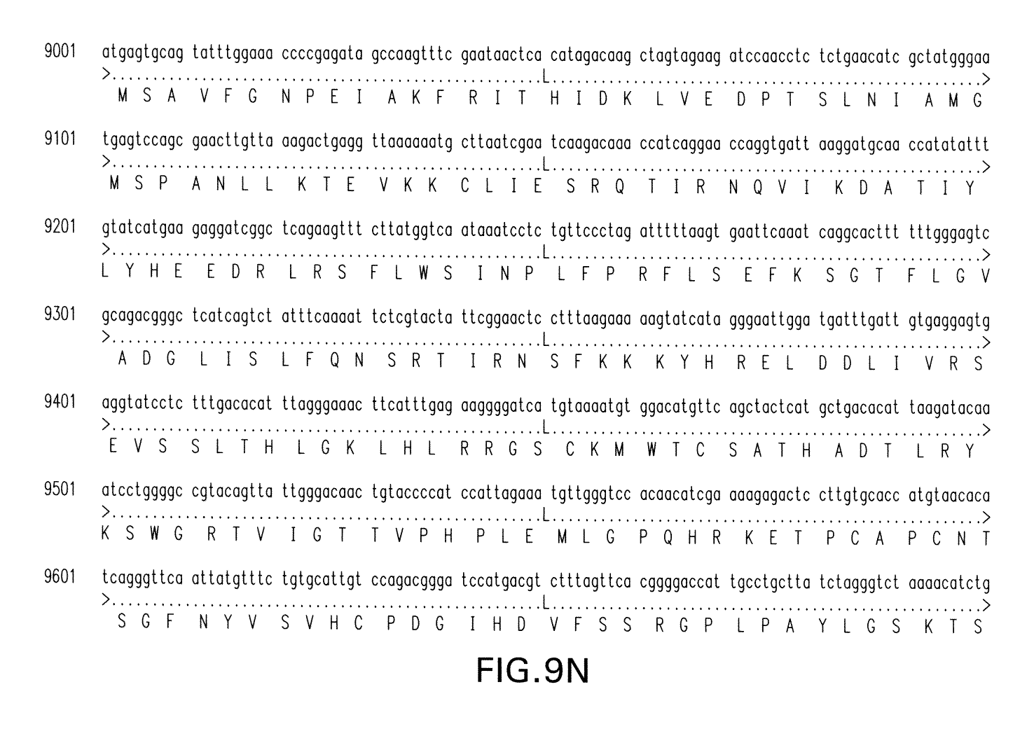

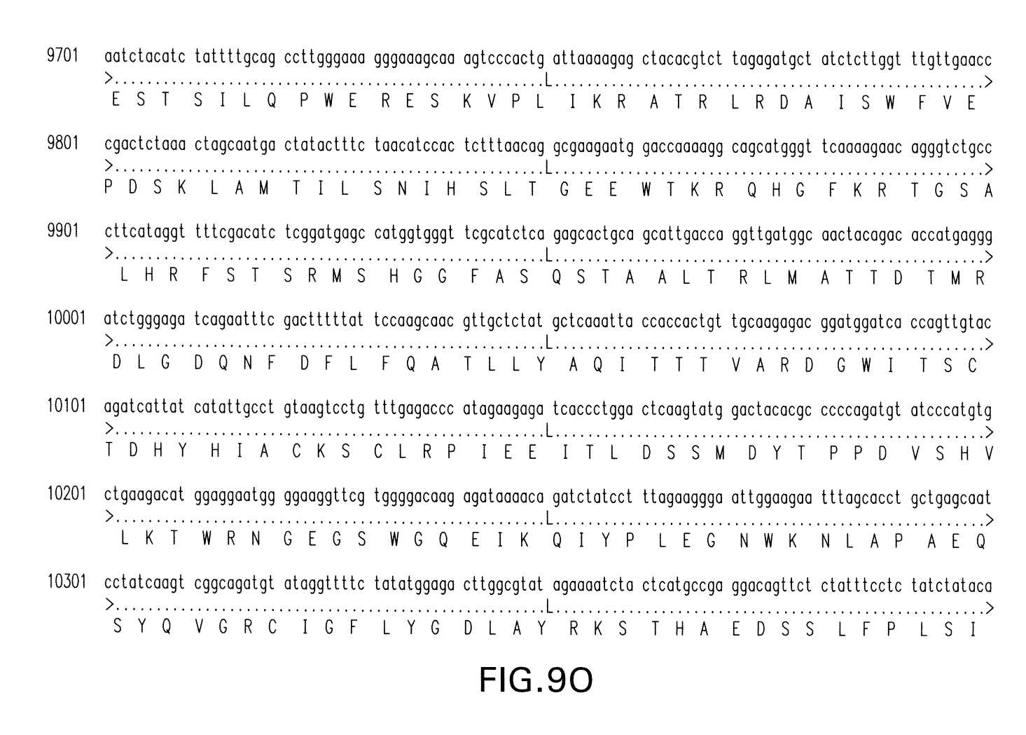

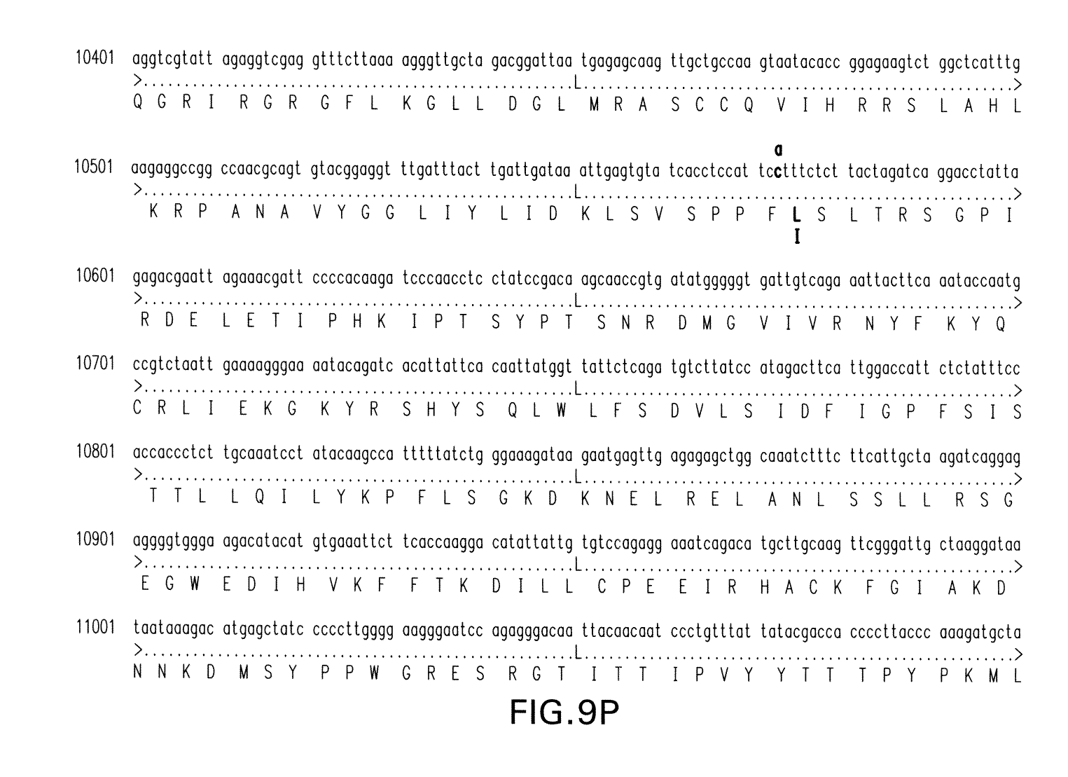

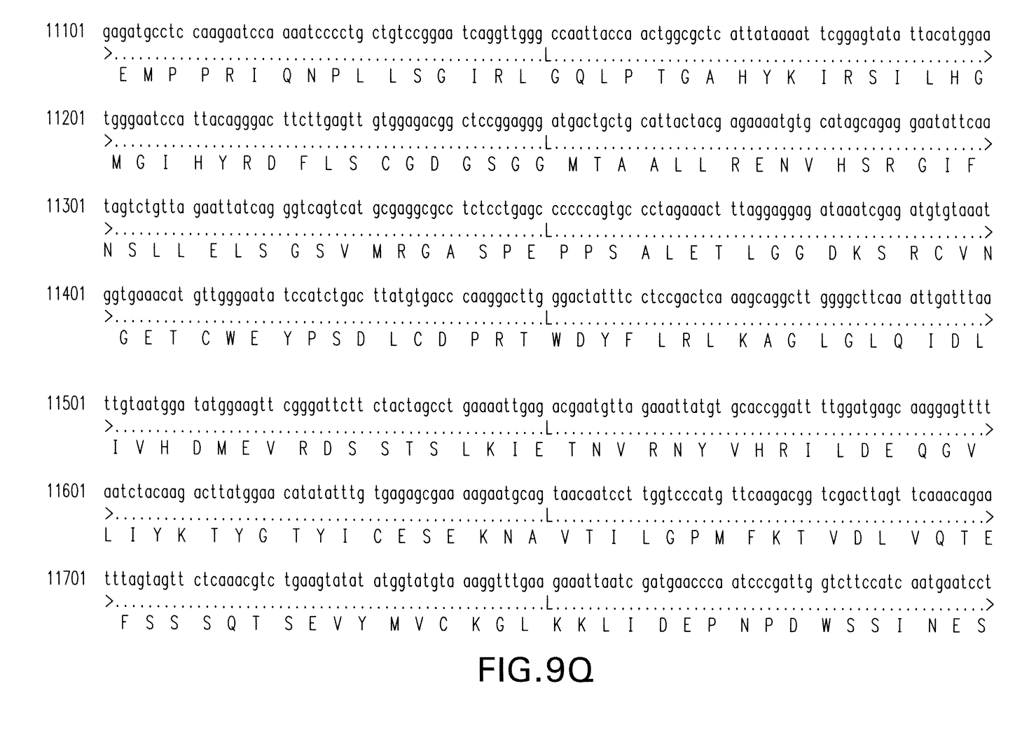

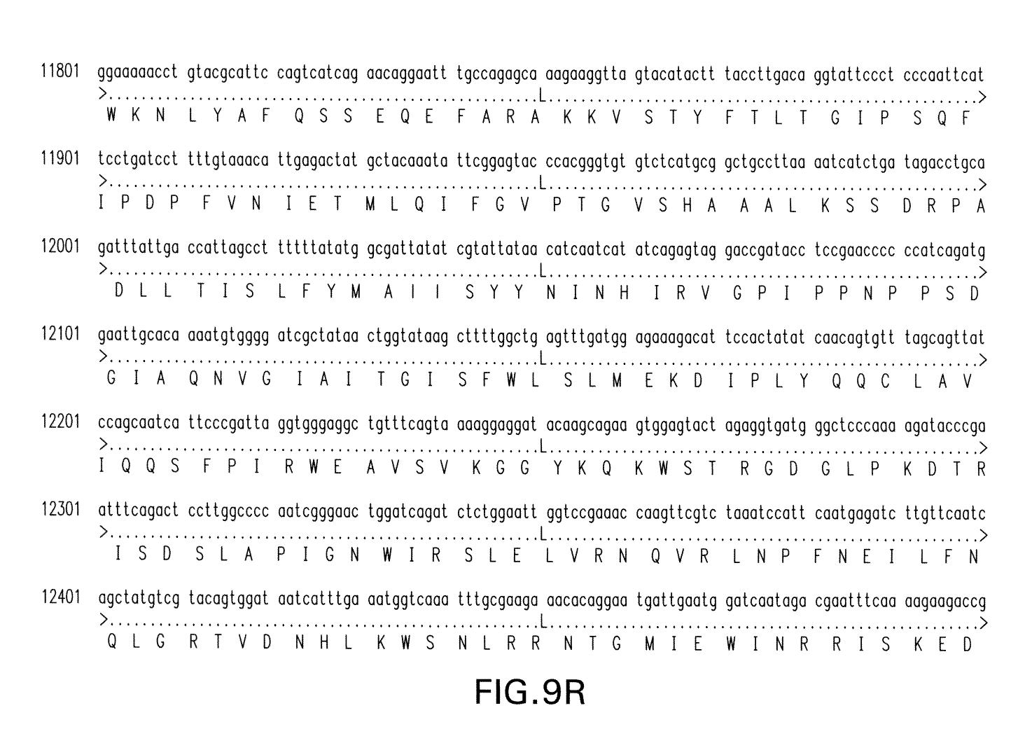

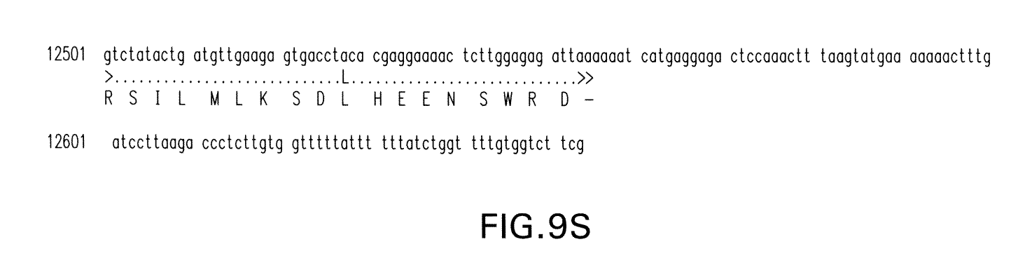

FIG. 1 illustrates the genomic organization of wt VSV and attn VSVN4CT1-gag1. FIG. 2 is an outline of the experimental protocol used to serially passage virus in Vero cells. The viruses at every fifth passage were analyzed by indicated assays. FIG. 8A-8L shows the comparison of the nucleotide (NT) and amino acid (AA) sequences of original (passage 0 or P0) viruses and passage 25 of VSV Indiana serotype. The NT and AA substitutions in the passaged virus are shown in bold. FIG. 9A through 9M shows the comparison of the nucleotide (NT) and amino acid (AA) sequences of original (passage 0 or P0) viruses and passage 25 of VSV New Jersey serotype. The NT and AA substitutions in the passaged virus are shown in bold. These sequences are summarized in Table 5 and in the sequence listing.

The attenuated rVSV_{IN}N4CT1Gag1 was used as the starting material for passaging in Vero stationary culture. Vero cells in T25 flasks were inoculated with the attenuated virus at a multiplicity of infection of 0.01 in DMEM (serum-free, antibiotic-free). The flasks were incubated at 32° C. for 2 to 3 days when the viral cytopathic effect (CPE) was evident in 90-100% of the cell monolayers. The culture medium was harvested, clarified by low speed centrifugation (1500 rpm, 10 min). After addition of 1× Sucrose Phosphate (SP) buffer (final concentration), the clarified virus was flash-frozen in a dry-ice ethanol bath and stored at ≦−60° C. This virus was labeled as passage 1 or P1 of rrVSV_{IN}N4CT1Gag. This virus was similarly used to inoculate fresh T25 flasks of Vero cells at moi of 0.01 to produce P2. This process, called serial passaging, was repeated 25 times to make P1 to P25 viruses. At each passage, the virus was titered by infectivity assay performed on a Vero monolayer.

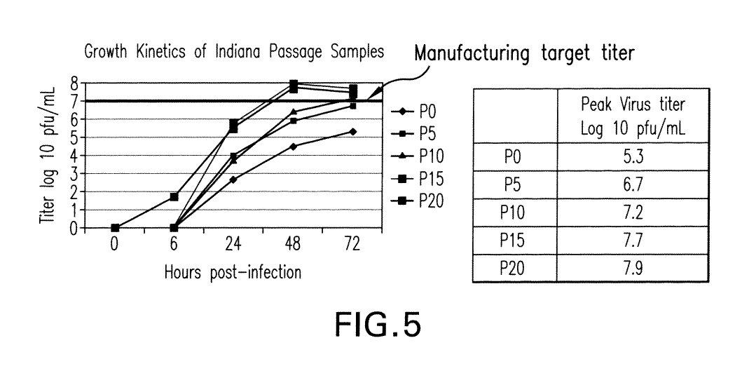

Accrual of amino acid substitutions following serial passages using the VSVin serotype is shown in FIG. 3. Growth kinetics of the passaged VSVin virus at MOI of 0.01 in Vero cells is shown in FIG. 5.

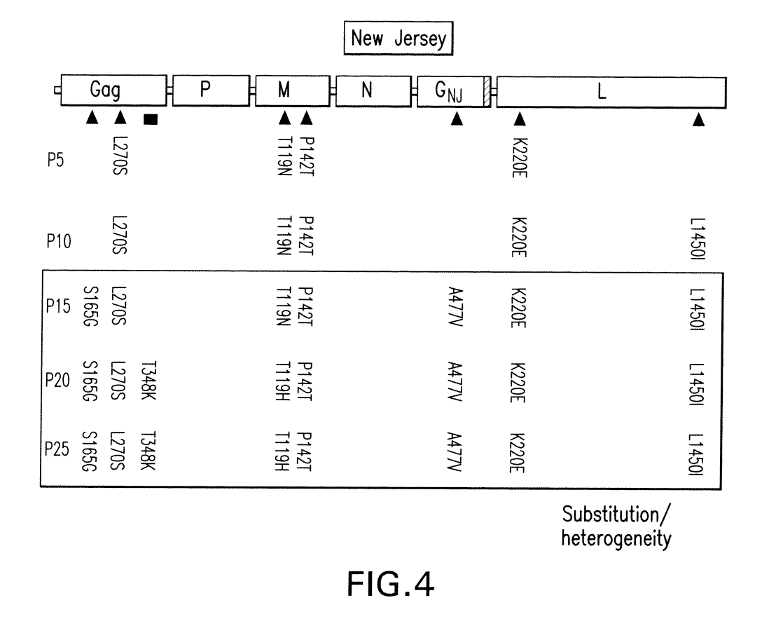

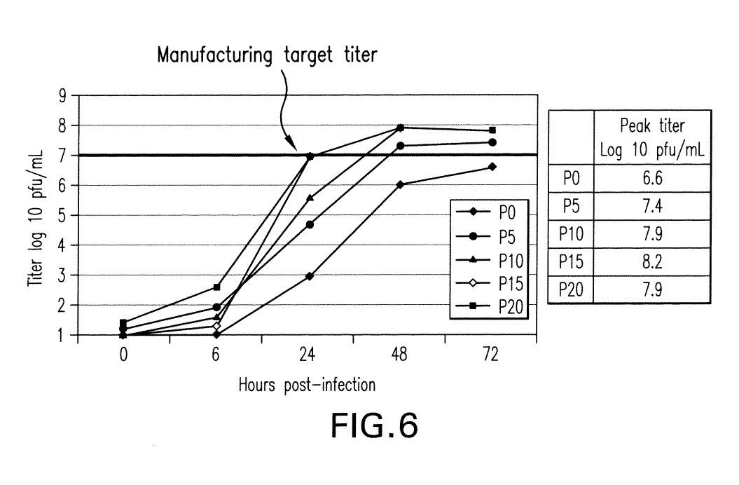

Accrual of amino acid substitutions following serial passages using the VSVnj serotype is shown in FIG. 4. Growth kinetics of the passaged VSVnj virus at MOI of 0.01 in Vero cells is shown in FIG. 6.

### Example 2

### Production of Recombinant VSVN4CT1GAG1

The tissue culture-adapted San Juan strain of the VSV Indian serotype (VSVin) and its corresponding genomic cDNA were provided by Dr. John K. Rose of Yale University, New Haven, Conn. and were used in the derivation of the rVSVN4CT1gag1 recombinants.

A detailed procedure for preparation of rVSVinN4CT1gag1 plasmid DNA has been described earlier (Clarke et al., J Virology, 81, 2056-64, 2007 and Cooper et al., J Virology, 82:207-29, 2008). The analogous NJ serotype glycoprotein vector, rVSVnjN4CT1gag1 was generated by replacing the Gin gene with truncated form of the Gnj gene and has been described in Cooper et al, 2008. FIG. 1 depicts schematically the order of viral genes within viral genomes for the attenuated VSV recombinants derived from wt VSV.

Infectious virus was recovered from genomic cDNA following transfection of Vero cells with the viral genome plasmid containing full-length genome and the five expression plasmids individually encoding the VSV N, P, L, M and G proteins. Expression from these plasmids is under the control of the T7 RNA polymerase promoter. The polymerase was supplied by electroporation of a plasmid encoding the T7 RNA polymerase under control of the human cytomegalovirus immediate early promoter/enhancer region. Rescued rVSV was purified by clonal isolation 3 to 4 times and amplified by passage in Vero cells 3 times. Virus rescue and all subsequent purification and amplification were performed in Vero cells. Fetal bovine serum (FBS) used in rescue of virus was of New Zealand origin. All steps after rescue were performed in serum-free medium (DMEM). All steps after clonal purification were performed in serum-free, antibiotic-free medium (DMEM).

Briefly, approximately 2.0×10⁷ Vero cells from a T150 flask were placed in a tube containing 10 μg of the rVSV_{IN}N4CT1Gag1 full-length viral cDNA plasmid and five support plasmids encoding the VSV N, P, L, M, and G proteins under control of the T7 promoter (10, 4, 1, 1, 2 μg, respectively). A seventh plasmid was added at 50 μg to supply T7 RNA polymerase; this plasmid is controlled by cellular RNA polymerase II. The suspension was subjected to electroporation and the cells were resuspended and transferred to a T150 flask. The flask was incubated at 37° C. for 3 hours; heat shocked at 43° C. for 5 hours and then incubated overnight at 37° C. Medium was replaced the following day and the flask was incubated at 32° C. on the second day. The flask was inspected each business day for signs of CPE. The flasks showing signs of CPE were followed until they achieved 80-100% CPE. At this point, the medium containing rescued virus (rescue supernatant) was harvested, supplemented with 1×SP¹ as a virus stabilizer, flash-frozen and stored at ≦−60° C. All rescue supernatants were screened for Gag expression by Western blot analysis using Gag-specific monoclonal antibodies; the rescued viruses showing Gag expression were subjected to viral genomic sequencing. One or more viral clones of each serotype having correct sequences were chosen for further plaque purification and amplification.

The virus plaquing was performed on Vero cells in six-well plates with agar overlay in DMEM with Gentamicin and without serum. The rescue supernatant containing infectious virus was diluted in DMEM so as to achieve ≦5 plaques per well. A number of well-isolated plaques were picked, suspended in DMEM and subjected to two to three additional rounds of plaque purification. The lead plaque from the fourth cloning was picked and suspended in DMEM and was then used for three successive virus amplifications on Vero cells in 6-well plates, T25 flasks and T150 flasks, respectively. Amplification was performed using DMEM without serum and without antibiotics. The recombinant virus obtained by this processed was termed as low passage virus and was used for further passaging in Vero cells for cell adaptation as described below.

### Example 3

### Experimental Protocol for Serial Passaging

Low passage VSV recombinants (P0) for each serotype were passaged 25 successive times on Vero cell monolayer in T-25 flasks as shown in FIG. 2. The Vero cells grown in serum-free medium were infected with virus at multiple of infection (MOI) of ˜0.01 and incubated in 32° C./5% CO₂ incubator until extensive CPE is visible, usually in 48 to 72 hours post-infection. After each amplification, the virus culture was clarified by centrifugation at low speed and stabilized with 1×SP Sucrose phosphate buffer. The 10×SP contains per liter of potassium phosphate, dibasic, 12.2 gm; potassium phosphate, monobasic, 5.17 gm; sucrose, 746.2 gm). Virus cultures from passages 1 to 25 were titered by plaque assay as described earlier (Clarke et al., J. Virol., 81: 2056-64, 2007. Nucleotide sequencing was performed for every fifth passage.

### Example 4

### Mice IC LD50 Study Results

### Mouse IC LD50 Studies

Young mice are highly sensitive to infection with VSV following intracranial inoculation (IC) causing rapid morbidity and mortality. The mouse IC LD50 neurovirulence animal model has been shown to be highly sensitive and capable of discriminating changes in virulence of VSV recombinants (Clarke et al, J. Virol., 81, 2056-64, 2007). Thus the mice receiving wt VSVin lost weight dramatically and died 2 to 4 days postinoculation with LD50 of 1 to 2 pfu. Viruses containing either CT truncations or gene shuffles, on the other hand, were shown to be more attenuated than the wt VSV with LD50 of 12 to 21 pfu (Clarke et al, J. Virol., 81, 2056-64, 2007). However, a dramatic decrease in virulence was seen when CT1 mutation was combined with gene shuffles (N4 and or gag1) mutations. For example, low passage rVSVinN4CT1gag1 exhibited extremely low level of virulence with LD50 of >10⁷ pfu, the highest dose tested in this animal model. The mice inoculated with this virus initially lost 10 to 20% of their weight post inoculation, but recovered to normal weight in ˜2 to 3 weeks. Similar results were seen with NJ serotype of the virus (Cooper et al., J Virology, 82, 207-29, 2008).

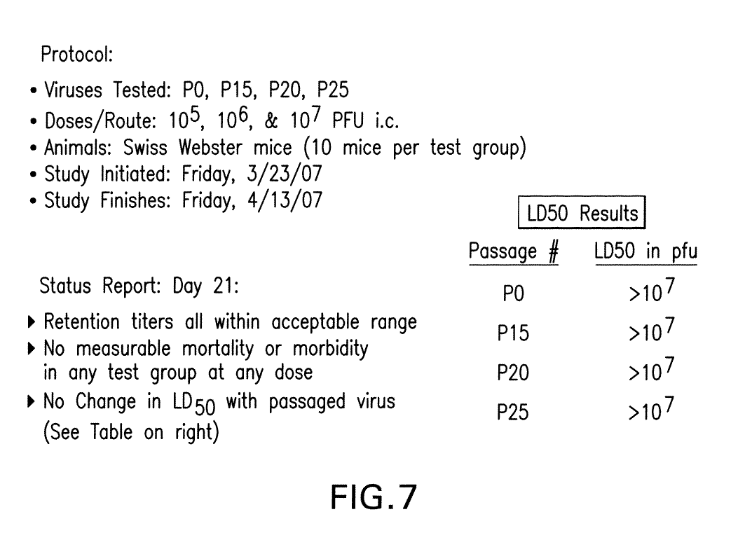

The Vero-adapted VSVN4CT1gag1 of this invention at passage 15 to 25 for each serotypes (IN and NJ) were tested in the murine IC LD50 animal model; no increase in virulence was seen, with LD50 of >10⁷ pfu for all passaged viruses as shown in FIG. 7. These results showed that the adaptive mutations accrued in viruses passaged in cell cultures did not impact on the virulence of the virus. The low virulence, along with their enhanced replication to high titers make the passaged viruses suitable for testing in human clinical trial.

### Method

The experimental details for the mouse IC LD50 studies are given in Clarke et al. (Clarke et al, J. Virol., 81, 2056-64, 2007) and Cooper et al. (Cooper et al., J Virology, 82, 207-29, 2008). Five-week old female Swiss Webster mice were anesthetized and injected IC with 10-fold dilutions of virus in 30 μl volume (10 animals per dilution, with dilutions made to range around the anticipated LD50). Weight and health status were recorded daily for two weeks. Mice becoming either bilaterally paralyzed or showing significant signs of distress or severe illness were sacrificed and recorded as succumbing to VSV disease. The LD50 was determined by the method of Reed and Muench, (Am. J. Hyg. 27, 493-97, 1938).

### SUMMARY

Compared to early passage rVSVN4CT1-gag1 viruses (P0), the serially passaged P15 viruses grew to much higher titers. This facilitated large scale manufacturing of clinical trial material (CTM). In addition, the stabilization of adaptive mutations by P15 provided viruses with manufacturing lot-to-lot consistency during CTM production. Moreover, the safety profile of the passaged viruses remained unchanged as tested by the murine IC LD50 animal model, thereby maintaining suitability for clinical evaluation.

### Example 5

### Scale-Up of Vectored HIV

A 10-L bioreactor (8-L working volume) containing Cytodex I microcarriers at 7.5 grams/L was inoculated with Vero cells at approximately 5×10⁵ cells/mL. The bioreactor was perfused with 0.5 culture volumes per day of Gibco VP-SFM media (with or without phenol red) at 37° C. After 70-90 hours or when the cell density was ≧2×10⁶ cells/mL, the culture was infected at an MOI of 0.001 at 32° C. with either low-passage (≦P5) or high-passage (P15) VSV N4CT1-gag1 virus. The cultures were sampled 2-3 times daily and ultimately harvested at 48-60 hours post-infection.

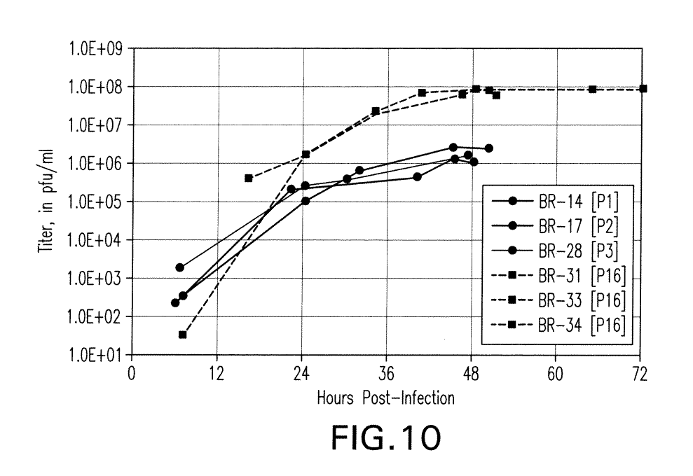

Scaling-up the Indiana serotype to 10-liter bioreactors, both low-passage (≦P5) and high-passage (P15) virus was tested. Bioreactor runs X-BRN10-VSV-14, 17, and 28 were completed using low-passage virus material (solid lines) to infect the cultures. Bioreactor runs X-BRN10-VSV-31, 33 and 34 were completed using high-passage Research Virus Seed (dashed lines). The growth kinetics for each run are shown in FIG. 10. The passage number of the virus is indicated in brackets in the legend of the figure.

For both the high- and low-passage runs, each set of runs is consistent with each other. The high-passage bioreactor runs produced up to two logs higher titers than the low-passage runs. Based upon the growth kinetics of the high-passage material, the harvest time was determined to be approximately 48 hours post-infection.

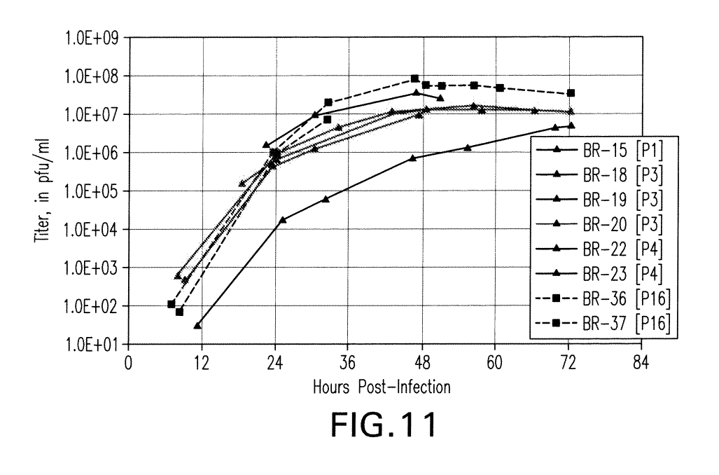

Scaling-up the New Jersey serotype to 10-liter bioreactors, again both low-passage (≦P5) and high-passage (P15) virus was tested. Bioreactor runs X-BRN10-VSV-15, 18, 19, 20, 22 and 23 were completed using low-passage virus material (solid lines) to infect the cultures. Bioreactor runs X-BRN10-VSV-36, and 37 were completed using high-passage Research Virus Seed (dashed lines). The growth kinetics for each run is shown in FIG. 11.

There were fewer differences between low-passage and high-passage runs for the New Jersey construct than were observed for the Indiana construct. Based upon the growth kinetics of the high passage material, the harvest time for VSVnjN4CT1-gag1 was determined to be approximately 48 hours post-infection, as was the case for VSVinN4CT1-gag1. In conclusion, the P15 N4CT1-gag1 resulted in ˜35-fold increase in bioreactor titers and a 45-fold increase in the number of virus particles produced per cell for Indiana and ˜5-fold increase in bioreactor titers and a 7-fold increase in the number of virus particles produced per cell for New Jersey. (See Tables 3 and 4)

TABLE 3
Comparison of VSVinN4CT1-gag1 Bioreactor Runs

| | | Culture | | Time of | | |
|---|---|---|---|---|---|---|
| | Number of | Volume | Cell Density @ | Harvest | Harvest Titer | Virus Particles |
| Run ID | Passages | (liters) | Infection (cells/ml) | (hpi) | (pfu/ml @ 50 hpi) | Per Cell |
| BR-14 | P1 | 8.0 | 2.96 × 10⁶ | 48 | 1.20 × 10⁶ | 0.4 |
| BR-17 | P2 | 8.0 | 3.07 × 10⁶ | 50 | 2.55 × 10⁶ | 0.8 |
| BR-28 | P3 | 7.0 | 2.78 × 10⁶ | 47 | 1.84 × 10⁶ | 0.7 |
| BR-31 | P16 | 8.0 | 2.25 × 10⁶ | 88 | 8.51 × 10⁷ | 37.8 |
| BR-33 | P16 | 8.0 | 2.24 × 10⁶ | 50 | 4.50 × 10⁷ | 20.1 |
| BR-34 | P16 | 8.0 | 1.99 × 10⁶ | 51 | 7.10 × 10⁷ | 35.7 |

TABLE 4
Comparison of VSVnjN4CT1-gag1 Bioreactor Runs

| | | Culture | | Time of | | Virus |
|---|---|---|---|---|---|---|
| | Number of | Volume | Cell Density @ | Harvest | | Particles Per |
| Run ID | Passages | (L) | Infection (cells/mL) | (hpi) | Peak Titer | Cell |
| BR-15 | P1 | 8.0 | 3.52 × 10⁶ | 72 | 5.28 × 10⁶ | 1.5 |
| BR-18 | P3 | 8.0 | 3.06 × 10⁶ | 72 | 1.72 × 10⁷ | 5.6 |
| BR-19 | P3 | 7.0 | 3.36 × 10⁶ | 66 | 1.46 × 10⁷ | 4.3 |
| BR-20 | P3 | 7.0 | 3.65 × 10⁶ | 66 | 7.26 × 10⁶ | 2.0 |
| BR-22 | P4 | 7.0 | 4.27 × 10⁶ | 50.5 | 3.41 × 10⁷ | 8.0 |
| BR-23 | P4 | 7.0 | 2.78 × 10⁶ | 47 | 1.07 × 10⁷ | 3.9 |
| BR-36 | P16 | 8.0 | 2.42 × 10⁶ | 72 | 5.57 × 10⁷ | 23.0 |
| BR-37 | P16 | 8.0 | 2.31 × 10⁶ | 50.3 | 8.36 × 10⁷ | 36.2 |

TABLE 5
Sequence description for VSV Indiana and New Jersey strains
after different passages (See FIGS. 8 and 9)

| SEQ ID NO | DESCRIPTION |
|---|---|
| 1 | VSV Indiana (gag1) DNA: passage number 25 |
| 2 | VSV Indiana (gag1) Protein: passage 0 |
| 3 | VSV Indiana (gag1) Protein: passage number 25 |
| 4 | VSV New Jersey (gag1) DNA: passage number 25 |
| 5 | VSV New Jersey (gag1) Protein: passage 0 |
| 6 | VSV New Jersey (gag1) Protein: passage number 25 |

What is claimed is:

1. An isolated, genetically modified vesicular stomatitis virus (VSV) having at least one amino acid mutation in a region corresponding to at least one of the following positions:
the amino acids at positions 119 or 142 of the M protein;
the amino acids at positions 109, 224, 438, 477, or 481 of the G protein; and
the amino acids at positions 205, 220 or 1450 of the L protein.

2. The genetically modified VSV of claim 1, wherein the nucleic acid encoding the genetically modified VSV further comprises a nucleic acid encoding at least one heterologous antigen, or a fragment thereof.

3. The genetically modified VSV of claim 1, wherein the one heterologous antigen, or a fragment thereof is from a pathogenic microorganism.

4. The genetically modified VSV of claim 3, wherein the pathogenic microorganism from which the nucleic acid encoding the heterologous antigen is obtained is selected from the group consisting of a virus, a bacterium, a protozoan and a fungus.

5. The genetically modified VSV of claim 4, wherein the heterologous antigen is selected from the group consisting of a human immunodeficiency virus (HIV) antigen, an HTLV antigen, an SIV antigen, an RSV antigen, a PIV antigen, an HSV antigen, a CMV antigen, an Epstein-Barr virus antigen, a Varicella-Zoster virus antigen, a mumps virus antigen, a measles virus antigen, an influenza virus antigen, a poliovirus antigen, a rhinovirus antigen, a hepatitis A virus antigen, a hepatitis B virus antigen, a hepatitis C virus antigen, a Norwalk virus antigen, a togavirus antigen, an alphavirus antigen, a rubella virus antigen, a rabies virus antigen, a Marburg virus antigen, an Ebola virus antigen, a papilloma virus antigen, a polyoma virus antigen, a metapneumovirus antigen, a coronavirus antigen, a Vibrio cholerae antigen, a Plasmodium falciparum antigen, a Plasmodium vivax antigen, a Plasmodium ovate antigen, a Plasmodium malariae antigen, a Plasmodium knowlesi antigen, a Streptococcus pneumoniae antigen, Streptococcus pyogenes antigen, a Helicobacter pylori antigen, a Streptococcus agalactiae antigen, a Neisseria meningitidis antigen, a Neisseria gonorrhoeae antigen, a Corynebacterium diphtheriae antigen, a Clostridium tetani antigen, a Bordetella pertussis antigen, a Haemophilus antigen, a Chlamydia antigen and an Escherichia coli antigen.

6. The genetically modified VSV of claim 5, wherein the heterologous antigen comprises an HIV protein.

7. The genetically modified VSV of claim 6, wherein the HIV protein is encoded by a gene selected from the group consisting of gag, env, pol, vif, nef, tat, vpr, rev and vpu.

8. The genetically modified VSV of claim 6, wherein the HIV protein is an HIV gag protein.

9. The genetically modified VSV of claim 8, wherein the HIV gag protein has at least one mutation at position 165, 270, 329, or 348.

10. The genetically modified VSV of claim 1, wherein the mutation comprises a conservative or non-conservative amino acid change.

11. The genetically modified VSV of claim 1, wherein the mutation is at either position 119 or 142 of the M protein or is at both positions 119 and 142 of the M protein.

12. The genetically modified VSV of claim 1, wherein the mutation of the amino acid at position 119 of the M protein is a T→N mutation and the mutation of the amino acid at position 142 of the M protein is a P→T mutation.

13. The genetically modified VSV of claim 1, wherein the mutation of the amino acids at position 109, 224, 438, 477 or 481 of the G protein is a K→N, N→T, S→I, A→V/G L, or V→I mutation, respectively.

14. The genetically modified VSV of claim 1, wherein the mutation of the amino acid at position 205, 220 or 1450 of the L protein is P→L, K→E, or L→I, respectively.

15. The genetically modified VSV of claim 9, wherein the mutation of amino acids at position 165, 270, 329 or 348 of the HIV gag protein is S→G, L→S, D→N or T→K, respectively.

16. The genetically modified VSV of claim 15, wherein the mutation in any one or more of the amino acids results in increased stability of the virus genotype and/or phenotype.

17. The genetically modified VSV of claim 16, wherein the mutation of any one or more of the amino acids further results in increased yield in virus production from a cell infected with said virus.

18. The genetically modified VSV of claim 1, further comprising at least two other mutations in its genome, the mutations selected from the group consisting of a temperature-sensitive mutation, a point mutation, a gene shuffling mutation, a G-stem mutation, a non-cytopathic M gene mutation, an ambisense RNA mutation, a truncated G gene mutation, a G gene insertion mutation and a gene deletion mutation.

19. A method for producing the genetically modified VSV of claim 1, the method comprising serial passaging of a VSV in a continuous mammalian cell line at a low multiplicity of infection (MOI) ranging from about 0.001 to about 0.1 plaque forming units (PFU)/ml for at least 5-15 passages, wherein the virus has a titer of at least 1×10⁶ PFU/ml and at least one or more of the mutations of any one of claims 1 through 14.

20. The method of claim 19, wherein the virus has a titer of at least 1×10⁷ PFU/ml.

21. The method of claim 20, wherein the cell line is a Vero, BHK, or 293 cell line.

22. The method of claim 21, wherein the method results in a 5 to 100 fold higher yield of virus compared to that obtained with a virus strain that has not been passaged for about 5 to 15 times at a low MOI ranging from about 0.001 to about 0.1 plaque forming units (PFU)/cell.

23. The method of claim 22, wherein the genetically modified VSV demonstrates an increase in stability of the virus genotype and/or phenotype.

24. An immunogenic composition comprising any one or more of the genetically modified VSV of claim 15 and a pharmaceutically acceptable carrier.

25. The immunogenic composition of claim 24, further comprising an adjuvant.

26. A method for protecting a mammal against infection with a pathogenic microorganism, the method comprising administering an immunologically effective amount of the genetically modified VSV of claim 15.

27. A method for protecting a mammal against infection with a pathogenic microorganism, the method comprising administering an immunologically effective amount of the immunogenic composition of claim 25.

28. A method for adapting a virus for growth in cell culture comprising
a. infecting the cell culture with the virus at a low multiplicity-of-infection (MOI) ranging from about 0.001 to about 0.1 plaque forming units (PFU) per cell;
b. harvesting the cell culture medium containing the virus;
c. clarifying the cell culture medium;
d. freezing the cell culture medium; and
e. repeating steps a) through d) for about 5 to about 15 times,
wherein the method results in a 5 to 100 fold increase in virus production/yield and an increase in the stability of the virus genotype and phenotype characteristics.

29. The method of claim 28, wherein the virus is an attenuated virus.

30. The method of claim 29, wherein the method allows for maintaining any pre-existing mutation(s) associated with virus attenuation.

31. The method of claim 29, wherein the method allows for maintaining a low neurovirulence profile associated with virus attenuation.

32. The method of claim 28, wherein the method is used for large scale production of an immunogenic composition.

33. The method of claim 32, wherein the method results in a 5 to 100 fold higher yield of virus compared to that obtained with a virus strain that has not been passaged for about 5 to 15 times at a low multiplicity of infection ranging from about 0.001 to about 0.1 plaque forming units per cell.

34. The method of claim 30, wherein the pre-existing mutation(s) associated with virus attenuation is selected from the group consisting of a temperature-sensitive mutation, a point mutation, a gene shuffling mutation, a G-stem mutation, a non-cytopathic M gene mutation, an ambisense RNA mutation, a truncated G gene mutation, a G gene insertion mutation and a gene deletion mutation.

35. The method of claim 29, wherein the attenuated virus is a strain of vesicular stomatitis virus (VSV).

36. The method of claim 30, wherein the VSV has at least one amino acid mutation in a region corresponding to at least one of the following positions:
the amino acids at positions 119 or 142 of the M protein;
the amino acids at positions 109, 224, 438, 477, or 481 of the G protein; and
the amino acids at positions 205, 220 or 1450 of the L protein.

37. The method of claim 36, wherein the mutation comprises a conservative or non-conservative amino acid change.

38. The method of claim 36, wherein the mutation is at either position 119 or 142 of the M protein or is at both positions 119 and 142 of the M protein.

39. The method of claim 38, wherein the mutation of the amino acid at position 119 of the M protein is a T→N mutation and the mutation of the amino acid at position 142 of the M protein is a P→T mutation.

40. The method of claim 36, wherein the mutation of the amino acids at position 109, 224, 438, 477 or 481 of the G protein is a K→N, N→T, S→I, (A→V/G→L), or V→I mutation, respectively.

41. The method of claim 36, wherein the mutation of the amino acid at position 205, 220 or 1450 of the L protein is P→L, K→E, or L→I, respectively.

42. The method of claim 35, wherein the strain of VSV is selected from the Indiana strain or the New Jersey strain or Isfahan serotype or other vesiculoviruses.

43. The method of claim 36, wherein the strain of VSV contains a nucleic acid encoding at least one heterologous antigen.

44. The method of claim 43, wherein the heterologous antigen is obtained from a pathogenic microorganism selected from the group consisting of a virus, a bacterium, a protozoan and a fungus.

45. The method of claim 43, wherein the heterologous antigen is selected from the group consisting of a human immunodeficiency virus (HIV) antigen, an HTLV antigen, an SIV antigen, an RSV antigen, a PIV antigen, an HSV antigen, a CMV antigen, an Epstein-Barr virus antigen, a Varicella-Zoster virus antigen, a mumps virus antigen, a measles virus antigen, an influenza virus antigen, a poliovirus antigen, a rhinovirus antigen, a hepatitis A virus antigen, a hepatitis B virus antigen, a hepatitis C virus antigen, a Norwalk virus antigen, a togavirus antigen, an alphavirus antigen, a rubella virus antigen, a rabies virus antigen, a Marburg virus antigen, an Ebola virus antigen, a papilloma virus antigen, a polyoma virus antigen, a metapneumovirus antigen, a coronavirus antigen, a Vibrio cholerae antigen, a Plasmodium falciparum antigen, a Plasmodium vivax antigen, a Plasmodium ovale antigen, a Plasmodium malariae antigen, a Plasmodium knowlesi antigen, a Streptococcus pneumoniae antigen, Streptococcus pyogenes antigen, a Helicobacter pylori antigen, a Streptococcus agalactiae antigen, a Neisseria meningitidis antigen, a Neisseria gonorrhoeae antigen, a Corynebacterium diphtheriae antigen, a Clostridium tetani antigen, a Bordetella pertussis antigen, a Haemophilus antigen, a Chlamydia antigen, and an Escherichia coli antigen.

46. The method of claim 43, wherein the heterologous antigen comprises an HIV protein.

47. The method of claim 46, wherein the HIV protein is encoded by a gene selected from the group consisting of gag, env, pol, vif, nef, tat, vpr, rev and vpu.

48. The method of claim 46, wherein the HIV protein is an HIV gag protein.

49. The method of claim 48, wherein the HIV gag protein has at least one mutation at position 165, 270, 329 or 348.

50. The method of claim 49, wherein the mutation of the amino acid at position 165, 270, 329 or 348 of the HIV gag protein is S→G, L→S, D→N, or T→K, respectively.


SEQUENCE LISTING (6 sequences)

SEQ ID NO: 1 - DNA, 15732 bp, Vesicular stomatitis virus
acgaagacaaacaaaccattattatcattaaaaggctcaggagaaactttaacagtaatcaaactcgagggatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaagaaccctttagagactatgtagaccggttctataaaactctaagagccgagcaagcttcacaggaggtaaaaaattggatgacagaaaccttgttggtccaaaatgcgaacccaaattgtaagactattttaaaagcattgggaccagcggctacactagaagaaatgatgacagcatgtcagggagtaggaggacccggccataaggcaagagttttggctgaagcaatgagccaagtaacaaattcagctaccataatgatgcagagaggcaattttaggaaccaaagaaagattgttaagtgtttcaattgtggcaaagaagggcacacagccagaaattgcagggcccctaggaaaaagggctgttggaaatgtggaaaggaaggacaccaaatgaaagattgtactgagagacaggctaattttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcagatcactctttggcaacgacccctcgtcacaataatcgctagcatattatgctacatatgaaaaaaactaacagatatcatggataatctcacaaaagttcgtgagtatctcaagtcctattctcgtctggatcaggcggtaggagagatagatgagatcgaagcacaacgagctgaaaagtccaattatgagttgttccaagaggatggagtggaagagcatactaagccctcttattttcaggcagcagatgattctgacacagaatctgaaccagaaattgaagacaatcaaggcttgtatgcaccagatccagaagctgagcaagttgaaggctttatacaggggcctttagatgactatgcagatgaggaagtggatgttgtatttacttcggactggaaacagcctgagcttgaatctgacgagcatggaaagaccttacggttgacatcgccagagggtttaagtggagagcagaaatcccagtggctttcgacgattaaagcagtcgtgcaaagtgccaaatactggaatctggcagagtgcacatttgaagcatcgggagaaggggtcattatgaaggagcgccagataactccggatgtatataaggtcactccagtgatgaacacacatccgtcccaatcagaagcagtatcagatgtttggtctctctcaaagacatccatgactttccaacccaagaaagcaagtcttcagcctctcaccatatccttggatgaattgttctcatctagaggagagttcatctctgtcggaggtgacggacgaatgtctcataaagaggccatcctgctcggcctgagatacaaaaagttgtacaatcaggcgagagtcaaatattctctgtagactatgaaaaaaagtaacagatatcacgatctaagtgttatcccaatccattcatcatgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagatggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacgctcactgcgaaggcagggcttatttgcaacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggactctatcggccacttcaaatgagctagtctaacttctagcttctgaacaatccccggtttactcagtctcccctaattccagcctctcgaacaactaatatcctgtcttttctatccctatgaaaaaaactaacagtaatcaaaatgtctgttacagtcaagagaatcattgacaacacagtcgtagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatgaccctataattctcagatcacctattatatattatgctacatatgaaaaaaactaacagagatcgatctgtttacgcgtcactatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcgagcagacggttggatgtgtcatgcttccaaatgggtcactacttgtgatttccgttggtatggaccgaagtatataacacattccatccgatccttcactccatctgtagaacaatgcaatgaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtacctactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagattttattttttggtgatactgggctatccaaaaatccaatcgagcttgtagaaggttggttcagtagttggaaaagctctattgcctcttttttctttatcatagggttaatcattgtactattcttggttctccgataagctagccgcctagccagattcttcatgtttggaccaaatcaacttgtgataccatgctcaaagaggcctcaattatatttgagtttttaatttttatgaaaaaaactaacagcaatcatggaagtccacgattttgagaccgacgagttcaatgatttcaatgaagatgactatgccacaagagaattcctgaatcccgatgagcgcatgacgtacttgaatcatgctgattacaacctgaattctcctctaattagtgatgatattgacaatttaatcaggaaattcaattctcttccaattccctcgatgtgggatagtaagaactgggatggagttcttgagatgttaacgtcatgtcaagccaatcccatcccaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttctcagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctaatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggagtatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcagatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagttcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttagcagttatccagcaatcattcccgattaggtgggaggctgtttcagtaaaaggaggatacaagcagaagtggagtactagaggtgatgggctcccaaaagatacccgaatttcagactccttggccccaatcgggaactggatcagatctctggaattggtccgaaaccaagttcgtctaaatccattcaatgagatcttgttcaatcagctatgtcgtacagtggataatcatttgaaatggtcaaatttgcgaagaaacacaggaatgattgaatggatcaatagacgaatttcaaaagaagaccggtctatactgatgttgaagagtgacctacacgaggaaaactcttggagagattaaaaaatcatgaggagactccaaactttaagtatgaaaaaaactttgatccttaagaccctcttgtggtttttatttttttatctggttttgtggtcttcgtgggtcggcatggcatctccacctcctcgcggtccgacctgggcatccgaaggaggacgtcgtccactcggatggctaagggaggggcccccgcggggctgctaacaaagcccgaaaggaagctgagttggctgctgccaccgctgagcaataactagcataaccccttggggcctctaaacgggtcttgaggggttttttgctgaaaggaggaactatatccggatcgagacctcgatactagtgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagg

SEQ ID NO: 2 - protein, 4007 aa, Vesicular stomatitis virus
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSYKGRPGNFLQSRPEPTAPPEESFRSGVETTTPPQKQEPIDKELYPLTSLRSLFGNDPSSQMDNLTKVREYLKSYSRLDQAVGEIDEIEAQRAEKSNYELFQEDGVEEHTKPSYFQAADDSDTESEPEIEDNQGLYAPDPEAEQVEGFIQGPLDDYADEEVDVVFTSDWKQPELESDEHGKTLRLTSPEGLSGEQKSQWLSTIKAVVQSAKYWNLAECTFEASGEGVIMKERQITPDVYKVTPVMNTHPSQSEAVSDVWSLSKTSMTFQPKKASLQPLTISLDELFSSRGEFISVGGDGRMSHKEAILLGLRYKKLYNQARVKYSLMSSLKKILGLKGKGKKSKKLGIAPPPYEEDTSMEYAPSAPIDKSYFGVDEMDTYDPNQLRYEKFFFTVKMTVRSNRPFRTYSDVAAAVSHWDHMYIGMAGKRPFYKILAFLGSSNLKATPAVLADQGQPEYHAHCEGRAYLPHRMGKTPPMLNVPEHFRRPFNIGLYKGTIELTMTIYDDESLEAAPMIWDHFNSSKFSDFREKALMFGLIVEKKASGAWVLDSIGHFKMSVTVKRIIDNTVVVPKLPANEDPVEYPADYFRKSKEIPLYINTTKSLSDLRGYVYQGLKSGNVSIIHVNSYLYGALKDIRGKLDKDWSSFGINIGKAGDTIGIFDLVSLKALDGVLPDGVSDASRTSADDKWLPLYLLGLYRVGRTQMPEYRKKLMDGLTNQCKMINEQFEPLVPEGRDIFDVWGNDSNYTKIVAAVDMFFHMFKKHECASFRYGTIVSRFKDCAALATFGHLCKITGMSTEDVTTWILNREVADEMVQMMLPGQEIDKADSYMPYLIDFGLSSKSPYSSVKNPAFHFWGQLTALLLRSTRARNARQPDDIEYTSLTTAGLLYAYAVGSSADLAQQFCVGDNKYTPDDSTGGLTTNAPPQGRDVVEWLGWFEDQNRKPTPDMMQYAKRAVMSLQGLREKTIGKYAKSEFDKKCLLYLAFLFIGVNCKFTIVFPHNQKGNWKNVPSNYHYCPSSSDLNWHNDLIGTALQVKMPKSHKAIRADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRMEVHDFETDEFNDFNEDDYATREFLNPDERMTYLNHADYNLNSPLISDDIDNLIRKFNSLPIPSMWDSKNWDGVLEMLTSCQANPIPTSQMHKWMGSWLMSDNHDASQGYSFLHEVDKEAEITFDVVETFIRGWGNKPIEYIKKERWTDSFKILAYLCQKFLDLHKLTLILNAVSEVELLNLARTFKGKVRRSSHGTNICRIRVPSLGPTFISEGWAYFKKLDILMDRNFLLMVKDVIIGRMQTVLSMVCRIDNLFSEQDIFSLLNIYRIGDKIVERQGNFSYDLIKMVEPICNLKLMKLARESRPLVPQFPHFENHIKTSVDEGAKIDRGIRFLHDQIMSVKTVDLTLVIYGSFRHWGHPFIDYYTGLEKLHSQVTMKKDIDVSYAKALASDLARIVLFQQFNDHKKWFVNGDLLPHDHPFKSHVKENTWPTAAQVQDFGDKWHELPLIKCFEIPDLLDPSIIYSDKSHSMNRSEVLKHVRMNPNTPIPSKKVLQTMLDTKATNWKEFLKEIDEKGLDDDDLIIGLKGKERELKLAGRFFSLMSWKLREYFVITEYLIKTHFVPMFKGLTMADDLTAVIKKMLDSSSGQGLKSYEAICIANHIDYEKWNNHQRKLSNGPVFRVMGQFLGYPSLIERTHEFFEKSLIYYNGRPDLMRVHNNTLINSTSQRVCWQGQEGGLEGLRQKGWSILNLLVIQREAKIRNTAVKVLAQGDNQVICTQYKTKKSRNVVELQGALNQMVSNNEKIMTAIKIGTGKLGLLINDDETMQSADYLNYGKIPIFRGVIRGLETKRWSRVTCVTNDQIPTCANIMSSVSTNALTVAHFAENPINAMIQYNYFGTFARLLLMMHDPALRQSLYEVQDKIPGLHSSTFKYAMLYLDPSIGGVSGMSLSRFLIRAFPDPVTESLSFWRFIHVHARSEHLKEMSAVFGNPEIAKFRITHIDKLVEDPTSLNIAMGMSPANLLKTEVKKCLIESRQTIRNQVIKDATIYLYHEEDRLRSFLWSINPLFPRFLSEFKSGTFLGVADGLISLFQNSRTIRNSFKKKYHRELDDLIVRSEVSSLTHLGKLHLRRGSCKMWTCSATHADTLRYKSWGRTVIGTTVPHPLEMLGPQHRKETPCAPCNTSGFNYVSVHCPDGIHDVFSSRGPLPAYLGSKTSESTSILQPWERESKVPLIKRATRLRDAISWFVEPDSKLAMTILSNIHSLTGEEWTKRQHGFKRTGSALHRFSTSRMSHGGFASQSTAALTRLMATTDTMRDLGDQNFDFLFQATLLYAQITTTVARDGWITSCTDHYHIACKSCLRPIEEITLDSSMDYTPPDVSHVLKTWRNGEGSWGQEIKQIYPLEGNWKNLAPAEQSYQVGRCIGFLYGDLAYRKSTHAEDSSLFPLSIQGRIRGRGFLKGLLDGLMRASCCQVIHRRSLAHLKRPANAVYGGLIYLIDKLSVSPPFLSLTRSGPIRDELETIPHKIPTSYPTSNRDMGVIVRNYFKYQCRLIEKGKYRSHYSQLWLFSDVLSIDFIGPFSISTTLLQILYKPFLSGKDKNELRELANLSSLLRSGEGWEDIHVKFFTKDILLCPEEIRHACKFGIAKDNNKDMSYPPWGRESRGTITTIPVYYTTTPYPKMLEMPPRIQNPLLSGIRLGQLPTGAHYKIRSILHGMGIHYRDFLSCGDGSGGMTAALLRENVHSRGIFNSLLELSGSVMRGASPEPPSALETLGGDKSRCVNGETCWEYPSDLCDPRTWDYFLRLKAGLGLQIDLIVMDMEVRDSSTSLKIETNVRNYVHRILDEQGVLIYKTYGTYICESEKNAVTILGPMFKTVDLVQTEFSSSQTSEVYMVCKGLKKLIDEPNPDWSSINESWKNLYAFQSSEQEFARAKKVSTYFTLTGIPSQFIPDPFVNIETMLQIFGVPTGVSHAAALKSSDRPADLLTISLFYMAIISYYNINHIRVGPIPPNPPSDGIAQNVGIAITGISFWLSLMEKDIPLYQQCLAVIQQSFPIRWEAVSVKGGYKQKWSTRGDGLPKDTRISDSLAPIGNWIRSLELVRNQVRLNPFNEILFNQLCRTVDNHLKWSNLRRNTGMIEWINRRISKEDRSILMLKSDLHEENSWRD

SEQ ID NO: 3 - protein, 4007 aa, Vesicular stomatitis virus
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPNCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSYKGRPGNFLQSRPEPTAPPEESFRSGVETTTPPQKQEPIDKELYPLTSLRSLFGNDPSSQMDNLTKVREYLKSYSRLDQAVGEIDEIEAQRAEKSNYELFQEDGVEEHTKPSYFQAADDSDTESEPEIEDNQGLYAPDPEAEQVEGFIQGPLDDYADEEVDVVFTSDWKQPELESDEHGKTLRLTSPEGLSGEQKSQWLSTIKAVVQSAKYWNLAECTFEASGEGVIMKERQITPDVYKVTPVMNTHPSQSEAVSDVWSLSKTSMTFQPKKASLQPLTISLDELFSSRGEFISVGGDGRMSHKEAILLGLRYKKLYNQARVKYSLMSSLKKILGLKGKGKKSKKLGIAPPPYEEDTSMEYAPSAPIDKSYFGVDEMDTYDPNQLRYEKFFFTVKMTVRSNRPFRTYSDVAAAVSHWDHMYIGMAGKRPFYKILAFLGSSNLKATPAVLADQGQPEYHAHCEGRAYLQHRMGKTPPMLNVPEHFRRPFNIGLYKGTIELTMTIYDDESLEAAPMIWDHFNSSKFSDFREKALMFGLIVEKKASGAWVLDSIGHFKMSVTVKRIIDNTVVVPKLPANEDPVEYPADYFRKSKEIPLYINTTKSLSDLRGYVYQGLKSGNVSIIHVNSYLYGALKDIRGKLDKDWSSFGINIGKAGDTIGIFDLVSLKALDGVLPDGVSDASRTSADDKWLPLYLLGLYRVGRTQMPEYRKKLMDGLTNQCKMINEQFEPLVPEGRDIFDVWGNDSNYTKIVAAVDMFFHMFKKHECASFRYGTIVSRFKDCAALATFGHLCKITGMSTEDVTTWILNREVADEMVQMMLPGQEIDKADSYMPYLIDFGLSSKSPYSSVKNPAFHFWGQLTALLLRSTRARNARQPDDIEYTSLTTAGLLYAYAVGSSADLAQQFCVGDNKYTPDDSTGGLTTNAPPQGRDVVEWLGWFEDQNRKPTPDMMQYAKRAVMSLQGLREKTIGKYAKSEFDKKCLLYLAFLFIGVNCKFTIVFPHNQKGNWKNVPSNYHYCPSSSDLNWHNDLIGTALQVKMPKSHKAIRADGWMCHASKWVTTCDFRWYGPKYITHSIRSFTPSVEQCNESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSTYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDEILFFGDTGLSKNPIELVEGWFSSWKSSIASFFFIIGLIIVLFLILRMEVHDFETDEFNDFNEDDYATREFLNPDERMTYLNHADYNLNSPLISDDIDNLIRKFNSLPIPSMWDSKNWDGVLEMLTSCQANPIPTSQMHKWMGSWLMSDNHDASQGYSFLHEVDKEAEITFDVVETFIRGWGNKPIEYIKKERWTDSFKILAYLCQKFLDLHKLTLILNAVSEVELLNLARTFKGKVRRSSHGTNICRIRVLSLGPTFISEGWAYFKKLDILMDRNFLLMVKDVIIGRMQTVLSMVCRIDNLFSEQDIFSLLNIYRIGDKIVERQGNFSYDLIKMVEPICNLKLMKLARESRPLVPQFPHFENHIKTSVDEGAKIDRGIRFLHDQIMSVKTVDLTLVIYGSFRHWGHPFIDYYTGLEKLHSQVTMKKDIDVSYAKALASDLARIVLFQQFNDHKKWFVNGDLLPHDHPFKSHVKENTWPTAAQVQDFGDKWHELPLIKCFEIPDLLDPSIIYSDKSHSMNRSEVLKHVRMNPNTPIPSKKVLQTMLDTKATNWKEFLKEIDEKGLDDDDLIIGLKGKERELKLAGRFFSLMSWKLREYFVITEYLIKTHFVPMFKGLTMADDLTAVIKKMLDSSSGQGLKSYEAICIANHIDYEKWNNHQRKLSNGPVFRVMGQFLGYPSLIERTHEFFEKSLIYYNGRPDLMRVHNNTLINSTSQRVCWQGQEGGLEGLRQKGWSILNLLVIQREAKIRNTAVKVLAQGDNQVICTQYKTKKSRNVVELQGALNQMVSNNEKIMTAIKIGTGKLGLLINDDETMQSADYLNYGKIPIFRGVIRGLETKRWSRVTCVTNDQIPTCANIMSSVSTNALTVAHFAENPINAMIQYNYFGTFARLLLMMHDPALRQSLYEVQDKIPGLHSSTFKYAMLYLDPSIGGVSGMSLSRFLIRAFPDPVTESLSFWRFIHVHARSEHLKEMSAVFGNPEIAKFRITHIDKLVEDPTSLNIAMGMSPANLLKTEVKKCLIESRQTIRNQVIKDATIYLYHEEDRLRSFLWSINPLFPRFLSEFKSGTFLGVADGLISLFQNSRTIRNSFKKKYHRELDDLIVRSEVSSLTHLGKLHLRRGSCKMWTCSATHADTLRYKSWGRTVIGTTVPHPLEMLGPQHRKETPCAPCNTSGFNYVSVHCPDGIHDVFSSRGPLPAYLGSKTSESTSILQPWERESKVPLIKRATRLRDAISWFVEPDSKLAMTILSNIHSLTGEEWTKRQHGFKRTGSALHRFSTSRMSHGGFASQSTAALTRLMATTDTMRDLGDQNFDFLFQATLLYAQITTTVARDGWITSCTDHYHIACKSCLRPIEEITLDSSMDYTPPDVSHVLKTWRNGEGSWGQEIKQIYPLEGNWKNLAPAEQSYQVGRCIGFLYGDLAYRKSTHAEDSSLFPLSIQGRIRGRGFLKGLLDGLMRASCCQVIHRRSLAHLKRPANAVYGGLIYLIDKLSVSPPFLSLTRSGPIRDELETIPHKIPTSYPTSNRDMGVIVRNYFKYQCRLIEKGKYRSHYSQLWLFSDVLSIDFIGPFSISTTLLQILYKPFLSGKDKNELRELANLSSLLRSGEGWEDIHVKFFTKDILLCPEEIRHACKFGIAKDNNKDMSYPPWGRESRGTITTIPVYYTTTPYPKMLEMPPRIQNPLLSGIRLGQLPTGAHYKIRSILHGMGIHYRDFLSCGDGSGGMTAALLRENVHSRGIFNSLLELSGSVMRGASPEPPSALETLGGDKSRCVNGETCWEYPSDLCDPRTWDYFLRLKAGLGLQIDLIVMDMEVRDSSTSLKIETNVRNYVHRILDEQGVLIYKTYGTYICESEKNAVTILGPMFKTVDLVQTEFSSSQTSEVYMVCKGLKKLIDEPNPDWSSINESWKNLYAFQSSEQEFARAKKVSTYFTLTGIPSQFIPDPFVNIETMLQIFGVPTGVSHAAALKSSDRPADLLTISLFYMAIISYYNINHIRVGPIPPNPPSDGIAQNVGIAITGISFWLSLMEKDIPLYQQCLAVIQQSFPIRWEAVSVKGGYKQKWSTRGDGLPKDTRISDSLAPIGNWIRSLELVRNQVRLNPFNEILFNQLCRTVDNHLKWSNLRRNTGMIEWINRRISKEDRSILMLKSDLHEENSWRD

SEQ ID NO: 4 - DNA, 15752 bp, Vesicular stomatitis virus
acgaagacaaacaaaccattattatcattaaaaggctcaggagaaactttaacagtaatcaaactcgagggatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcggcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggatcaaataaaatagtaagaatgtatagccctaccagcattctggacataagacaaggaccaaaagaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttggctgaagcaatgagccaagtaacaaattcagctaccataatgatgcagagaggcaattttaggaaccaaagaaagattgttaagtgtttcaattgtggcaaagaagggcacacagccagaaattgcagggcccctaggaaaaagggctgttggaaatgtggaaaggaaggacaccaaatgaaagattgtactgagagacaggctaattttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcagatcactctttggcaacgacccctcgtcacaataatcgctagcatattatgctacatatgaaaaaaactaacagatatcatggataatctcacaaaagttcgtgagtatctcaagtcctattctcgtctggatcaggcggtaggagagatagatgagatcgaagcacaacgagctgaaaagtccaattatgagttgttccaagaggatggagtggaagagcatactaagccctcttattttcaggcagcagatgattctgacacagaatctgaaccagaaattgaagacaatcaaggcttgtatgcaccagatccagaagctgagcaagttgaaggctttatacaggggcctttagatgactatgcagatgaggaagtggatgttgtatttacttcggactggaaacagcctgagcttgaatctgacgagcatggaaagaccttacggttgacatcgccagagggtttaagtggagagcagaaatcccagtggctttcgacgattaaagcagtcgtgcaaagtgccaaatactggaatctggcagagtgcacatttgaagcatcgggagaaggggtcattatgaaggagcgccagataactccggatgtatataaggtcactccagtgatgaacacacatccgtcccaatcagaagcagtatcagatgtttggtctctctcaaagacatccatgactttccaacccaagaaagcaagtcttcagcctctcaccatatccttggatgaattgttctcatctagaggagagttcatctctgtcggaggtgacggacgaatgtctcataaagaggccatcctgctcggcctgagatacaaaaagttgtacaatcaggcgagagtcaaatattctctgtagactatgaaaaaaagtaacagatatcacgatctaagtgttatcccaatccattcatcatgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagatggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccaatccagcggtattggcagatcaaggtcaaccagagtatcacgctcactgcgaaggcagggcttatttgacacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggactctatcggccacttcaaatgagctagtctaacttctagcttctgaacaatccccggtttactcagtctcccctaattccagcctctcgaacaactaatatcctgtcttttctatccctatgaaaaaaactaacagtaatcaaaatgtctgttacagtcaagagaatcattgacaacacagtcgtagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatgaccctataattctcagatcacctattatatattatgttacatatgaaaaaaactaacagagatcgatctgtttacgcgtcactatgttgtcttatctaatctttgcacttgccgtttcgcccattttgggcaaaattgaaattgtgtttcctcaacataccactggggattggaagagagttccccatgaatataattattgccctaccagcgcagacaagaactcacatgggactcaaacaggaatccctgttgagttaacaatgccaaaaggactaacaacccatcaagttgaaggatttatgtgtcactcagccttgtggatgaccacttgtgacttcagatggtatgggccaaaatacataacccattccatacataatgaagagcctacagattatcaatgtttggaggccattaagtcatacaaagatggagtcagtttcaatccagggtttcctcctcagagctgcgggtatggcacagttaccgatgccgaagcccatattgtgacagttactccccactctgtcaaagtggacgagtacacgggggaatggatcgatccacatttcatcggaggaaggtgcaaaggacaaatttgtgaaacagtccataattccacaaaatggtttacgtcctctgatggagaaagtgtctgcagtcaattgtttactttggttggaggaatttttttctctgattcagaagagattacctccatggggttaccagaaacaggaatcagaagtaattacttcccctacatatctacagagggaatttgcaaaatgccgttttgcagaaaacaggggtacaagcttaaaaatgacctctggttccagatcatggacccagacctggataaaacggttagagatctccctcatattaaggactgtgacctctcctcgtccataatcacaccaggagaacatgctacagacatctcactgatatcagatgttgaaaggatcctagactatgctctttgtcagaatacatggagtaaaattgaatcgggagaaccaattactccggtagatctcagctatcttgggccaaaaaacccaggggttgggccggtcttcaccatcattaacggttccctgcattattttacatcgaagtatctgcgagtcgaattagaaagtcctgtcatacccagaatggaaggaaaagttgcaggaactaggattgtacggcaattgtgggatcagtggtttcctttcggagaagttgagattggacccaatggtgtgttgaaaacgaagcaagggtataaattcccactacacatcattggaactggagaagtagacagtgacatcaaaatggaaagggttgtcaagcactgggaacacccccatattgaggccgctcagacatttttaaaaaaagatgacacaggagaagtcctttattatggcgacaccggagtgtcgaaaaatccagttgaattagtcgagggatggtttagtggatggaggagctccctcatgggagtgctggttgtgattataggatttgtgattttaatgtttttaattcgataagctagccgcctagccagattcttcatgtttggaccaaatcaacttgtgataccatgctcaaagaggcctcaattatatttgagtttttaatttttatgaaaaaaactaacagcaatcatggaagtccacgattttgagaccgacgagttcaatgatttcaatgaagatgactatgccacaagagaattcctgaatcccgatgagcgcatgacgtacttgaatcatgctgattacaacctgaattctcctctaattagtgatgatattgacaatttaatcaggaaattcaattctcttccaattccctcgatgtgggatagtaagaactgggatggagttcttgagatgttaacgtcatgtcaagccaatcccatcccaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcgagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggagtatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcagatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcatttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagttcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttagcagttatccagcaatcattcccgattaggtgggaggctgtttcagtaaaaggaggatacaagcagaagtggagtactagaggtgatgggctcccaaaagatacccgaatttcagactccttggccccaatcgggaactggatcagatctctggaattggtccgaaaccaagttcgtctaaatccattcaatgagatcttgttcaatcagctatgtcgtacagtggataatcatttgaaatggtcaaatttgcgaagaaacacaggaatgattgaatggatcaatagacgaatttcaaaagaagaccggtctatactgatgttgaagagtgacctacacgaggaaaactcttggagagattaaaaaatcatgaggagactccaaactttaagtatgaaaaaaactttgatccttaagaccctcttgtggtttttattttttatctggttttgtggtcttcgtgggtcggcatggcatctccacctcctcgcggtccgacctgggcatccgaaggaggacgtcgtccactcggatggctaagggaggggcccccgcggggctgctaacaaagcccgaaaggaagctgagttggctgctgccaccgctgagcaataactagcataaccccttggggcctctaaacgggtcttgaggggttttttgctgaaaggaggaactatatccggatcgagacctcgatactagtgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagg

SEQ ID NO: 5 - protein, 4015 aa, Vesicular stomatitis virus
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSYKGRPGNFLQSRPEPTAPPEESFRSGVETTTPPQKQEPIDKELYPLTSLRSLFGNDPSSQMDNLTKVREYLKSYSRLDQAVGEIDEIEAQRAEKSNYELFQEDGVEEHTKPSYFQAADDSDTESEPEIEDNQGLYAPDPEAEQVEGFIQGPLDDYADEEVDVVFTSDWKQPELESDEHGKTLRLTSPEGLSGEQKSQWLSTIKAVVQSAKYWNLAECTFEASGEGVIMKERQITPDVYKVTPVMNTHPSQSEAVSDVWSLSKTSMTFQPKKASLQPLTISLDELFSSRGEFISVGGDGRMSHKEAILLGLRYKKLYNQARVKYSLMSSLKKILGLKGKGKKSKKLGIAPPPYEEDTSMEYAPSAPIDKSYFGVDEMDTYDPNQLRYEKFFFTVKMTVRSNRPFRTYSDVAAAVSHWDHMYIGMAGKRPFYKILAFLGSSNLKATPAVLADQGQPEYHAHCEGRAYLPHRMGKTPPMLNVPEHFRRPFNIGLYKGTIELTMTIYDDESLEAAPMIWDHFNSSKFSDFREKALMFGLIVEKKASGAWVLDSIGHFKMSVTVKRIIDNTVVVPKLPANEDPVEYPADYFRKSKEIPLYINTTKSLSDLRGYVYQGLKSGNVSIIHVNSYLYGALKDIRGKLDKDWSSFGINIGKAGDTIGIFDLVSLKALDGVLPDGVSDASRTSADDKWLPLYLLGLYRVGRTQMPEYRKKLMDGLTNQCKMINEQFEPLVPEGRDIFDVWGNDSNYTKIVAAVDMFFHMFKKHECASFRYGTIVSRFKDCAALATFGHLCKITGMSTEDVTTWILNREVADEMVQMMLPGQEIDKADSYMPYLIDFGLSSKSPYSSVKNPAFHFWGQLTALLLRSTRARNARQPDDIEYTSLTTAGLLYAYAVGSSADLAQQFCVGDNKYTPDDSTGGLTTNAPPQGRDVVEWLGWFEDQNRKPTPDMMQYAKRAVMSLQGLREKTIGKYAKSEFDKMLSYLIFALAVSPILGKIEIVFPQHTTGDWKRVPHEYNYCPTSADKNSHGTQTGIPVELTMPKGLTTHQVEGFMCHSALWMTTCDFRWYGPKYITHSIHNEEPTDYQCLEAIKSYKDGVSFNPGFPPQSCGYGTVTDAEAHIVTVTPHSVKVDEYTGEWIDPHFIGGRCKGQICETVHNSTKWFTSSDGESVCSQLFTLVGGIFFSDSEEITSMGLPETGIRSNYFPYISTEGICKMPFCRKQGYKLKNDLWFQIMDPDLDKTVRDLPHIKDCDLSSSIITPGEHATDISLISDVERILDYALCQNTWSKIESGEPITPVDLSYLGPKNPGVGPVFTIINGSLHYFTSKYLRVELESPVIPRMEGKVAGTRIVRQLWDQWFPFGEVEIGPNGVLKTKQGYKFPLHIIGTGEVDSDIKMERVVKHWEHPHIEAAQTFLKKDDTGEVLYYGDTGVSKNPVELVEGWFSGWRSSLMGVLAVIIGFVILMFLIRMEVHDFETDEFNDFNEDDYATREFLNPDERMTYLNHADYNLNSPLISDDIDNLIRKFNSLPIPSMWDSKNWDGVLEMLTSCQANPIPTSQMHKWMGSWLMSDNHDASQGYSFLHEVDKEAEITFDVVETFIRGWGNKPIEYIKKERWTDSFKILAYLCQKFLDLHKLTLILNAVSEVELLNLARTFKGKVRRSSHGTNICRIRVPSLGPTFISEGWAYFKKLDILMDRNFLLMVKDVIIGRMQTVLSMVCRIDNLFSEQDIFSLLNIYRIGDKIVERQGNFSYDLIKMVEPICNLKLMKLARESRPLVPQFPHFENHIKTSVDEGAKIDRGIRFLHDQIMSVKTVDLTLVIYGSFRHWGHPFIDYYTGLEKLHSQVTMKKDIDVSYAKALASDLARIVLFQQFNDHKKWFVNGDLLPHDHPFKSHVKENTWPTAAQVQDFGDKWHELPLIKCFEIPDLLDPSIIYSDKSHSMNRSEVLKHVRMNPNTPIPSKKVLQTMLDTKATNWKEFLKEIDEKGLDDDDLIIGLKGKERELKLAGRFFSLMSWKLREYFVITEYLIKTHFVPMFKGLTMADDLTAVIKKMLDSSSGQGLKSYEAICIANHIDYEKWNNHQRKLSNGPVFRVMGQFLGYPSLIERTHEFFEKSLIYYNGRPDLMRVHNNTLINSTSQRVCWQGQEGGLEGLRQKGWSILNLLVIQREAKIRNTAVKVLAQGDNQVICTQYKTKKSRNVVELQGALNQMVSNNEKIMTAIKIGTGKLGLLINDDETMQSADYLNYGKIPIFRGVIRGLETKRWSRVTCVTNDQIPTCANIMSSVSTNALTVAHFAENPINAMIQYNYFGTFARLLLMMHDPALRQSLYEVQDKIPGLHSSTFKYAMLYLDPSIGGVSGMSLSRFLIRAFPDPVTESLSFWRFIHVHARSEHLKEMSAVFGNPEIAKFRITHIDKLVEDPTSLNIAMGMSPANLLKTEVKKCLIESRQTIRNQVIKDATIYLYHEEDRLRSFLWSINPLFPRFLSEFKSGTFLGVADGLISLFQNSRTIRNSFKKKYHRELDDLIVRSEVSSLTHLGKLHLRRGSCKMWTCSATHADTLRYKSWGRTVIGTTVPHPLEMLGPQHRKETPCAPCNTSGFNYVSVHCPDGIHDVFSSRGPLPAYLGSKTSESTSILQPWERESKVPLIKRATRLRDAISWFVEPDSKLAMTILSNIHSLTGEEWTKRQHGFKRTGSALHRFSTSRMSHGGFASQSTAALTRLMATTDTMRDLGDQNFDFLFQATLLYAQITTTVARDGWITSCTDHYHIACKSCLRPIEEITLDSSMDYTPPDVSHVLKTWRNGEGSWGQEIKQIYPLEGNWKNLAPAEQSYQVGRCIGFLYGDLAYRKSTHAEDSSLFPLSIQGRIRGRGFLKGLLDGLMRASCCQVIHRRSLAHLKRPANAVYGGLIYLIDKLSVSPPFLSLTRSGPIRDELETIPHKIPTSYPTSNRDMGVIVRNYFKYQCRLIEKGKYRSHYSQLWLFSDVLSIDFIGPFSISTTLLQILYKPFLSGKDKNELRELANLSSLLRSGEGWEDIHVKFFTKDILLCPEEIRHACKFGIAKDNNKDMSYPPWGRESRGTITTIPVYYTTTPYPKMLEMPPRIQNPLLSGIRLGQLPTGAHYKIRSILHGMGIHYRDFLSCGDGSGGMTAALLRENVHSRGIFNSLLELSGSVMRGASPEPPSALETLGGDKSRCVNGETCWEYPSDLCDPRTWDYFLRLKAGLGLQIDLIVMDMEVRDSSTSLKIETNVRNYVHRILDEQGVLIYKTYGTYICESEKNAVTILGPMFKTVDLVQTEFSSSQTSEVYMVCKGLKKLIDEPNPDWSSINESWKNLYAFQSSEQEFARAKKVSTYFTLTGIPSQFIPDPFVNIETMLQIFGVPTGVSHAAALKSSDRPADLLTISLFYMAIISYYNINHIRVGPIPPNPPSDGIAQNVGIAITGISFWLSLMEKDIPLYQQCLAVIQQSFPIRWEAVSVKGGYKQKWSTRGDGLPKDTRISDSLAPIGNWIRSLELVRNQVRLNPFNEILFNQLCRTVDNHLKWSNLRRNTGMIEWINRRISKEDRSILMLKSDLHEENSWRD

SEQ ID NO: 6 - protein, 4015 aa, Vesicular stomatitis virus
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFGPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGSNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSYKGRPGNFLQSRPEPTAPPEESFRSGVETTTPPQKQEPIDKELYPLTSLRSLFGNDPSSQMDNLTKVREYLKSYSRLDQAVGEIDEIEAQRAEKSNYELFQEDGVEEHTKPSYFQAADDSDTESEPEIEDNQGLYAPDPEAEQVEGFIQGPLDDYADEEVDVVFTSDWKQPELESDEHGKTLRLTSPEGLSGEQKSQWLSTIKAVVQSAKYWNLAECTFEASGEGVIMKERQITPDVYKVTPVMNTHPSQSEAVSDVWSLSKTSMTFQPKKASLQPLTISLDELFSSRGEFISVGGDGRMSHKEAILLGLRYKKLYNQARVKYSLMSSLKKILGLKGKGKKSKKLGIAPPPYEEDTSMEYAPSAPIDKSYFGVDEMDTYDPNQLRYEKFFFTVKMTVRSNRPFRTYSDVAAAVSHWDHMYIGMAGKRPFYKILAFLGSSNLKANPAVLADQGQPEYHAHCEGRAYLTHRMGKTPPMLNVPEHFRRPFNIGLYKGTIELTMTIYDDESLEAAPMIWDHFNSSKFSDFREKALMFGLIVEKKASGAWVLDSIGHFKMSVTVKRIIDNTVVVPKLPANEDPVEYPADYFRKSKEIPLYINTTKSLSDLRGYVYQGLKSGNVSIIHVNSYLYGALKDIRGKLDKDWSSFGINIGKAGDTIGIFDLVSLKALDGVLPDGVSDASRTSADDKWLPLYLLGLYRVGRTQMPEYRKKLMDGLTNQCKMINEQFEPLVPEGRDIFDVWGNDSNYTKIVAAVDMFFHMFKKHECASFRYGTIVSRFKDCAALATFGHLCKITGMSTEDVTTWILNREVADEMVQMMLPGQEIDKADSYMPYLIDFGLSSKSPYSSVKNPAFHFWGQLTALLLRSTRARNARQPDDIEYTSLTTAGLLYAYAVGSSADLAQQFCVGDNKYTPDDSTGGLTTNAPPQGRDVVEWLGWFEDQNRKPTPDMMQYAKRAVMSLQGLREKTIGKYAKSEFDKMLSYLIFALAVSPILGKIEIVFPQHTTGDWKRVPHEYNYCPTSADKNSHGTQTGIPVELTMPKGLTTHQVEGFMCHSALWMTTCDFRWYGPKYITHSIHNEEPTDYQCLEAIKSYKDGVSFNPGFPPQSCGYGTVTDAEAHIVTVTPHSVKVDEYTGEWIDPHFIGGRCKGQICETVHNSTKWFTSSDGESVCSQLFTLVGGIFFSDSEEITSMGLPETGIRSNYFPYISTEGICKMPFCRKQGYKLKNDLWFQIMDPDLDKTVRDLPHIKDCDLSSSIITPGEHATDISLISDVERILDYALCQNTWSKIESGEPITPVDLSYLGPKNPGVGPVFTIINGSLHYFTSKYLRVELESPVIPRMEGKVAGTRIVRQLWDQWFPFGEVEIGPNGVLKTKQGYKFPLHIIGTGEVDSDIKMERVVKHWEHPHIEAAQTFLKKDDTGEVLYYGDTGVSKNPVELVEGWFSGWRSSLMGVLVVIIGFVILMFLIRMEVHDFETDEFNDFNEDDYATREFLNPDERMTYLNHADYNLNSPLISDDIDNLIRKFNSLPIPSMWDSKNWDGVLEMLTSCQANPIPTSQMHKWMGSWLMSDNHDASQGYSFLHEVDKEAEITFDVVETFIRGWGNKPIEYIKKERWTDSFKILAYLCQKFLDLHKLTLILNAVSEVELLNLARTFKGKVRRSSHGTNICRIRVPSLGPTFISEGWAYFEKLDILMDRNFLLMVKDVIIGRMQTVLSMVCRIDNLFSEQDIFSLLNIYRIGDKIVERQGNFSYDLIKMVEPICNLKLMKLARESRPLVPQFPHFENHIKTSVDEGAKIDRGIRFLHDQIMSVKTVDLTLVIYGSFRHWGHPFIDYYTGLEKLHSQVTMKKDIDVSYAKALASDLARIVLFQQFNDHKKWFVNGDLLPHDHPFKSHVKENTWPTAAQVQDFGDKWHELPLIKCFEIPDLLDPSIIYSDKSHSMNRSEVLKHVRMNPNTPIPSKKVLQTMLDTKATNWKEFLKEIDEKGLDDDDLIIGLKGKERELKLAGRFFSLMSWKLREYFVITEYLIKTHFVPMFKGLTMADDLTAVIKKMLDSSSGQGLKSYEAICIANHIDYEKWNNHQRKLSNGPVFRVMGQFLGYPSLIERTHEFFEKSLIYYNGRPDLMRVHNNTLINSTSQRVCWQGQEGGLEGLRQKGWSILNLLVIQREAKIRNTAVKVLAQGDNQVICTQYKTKKSRNVVELQGALNQMVSNNEKIMTAIKIGTGKLGLLINDDETMQSADYLNYGKIPIFRGVIRGLETKRWSRVTCVTNDQIPTCANIMSSVSTNALTVAHFAENPINAMIQYNYFGTFARLLLMMHDPALRQSLYEVQDKIPGLHSSTFKYAMLYLDPSIGGVSGMSLSRFLIRAFPDPVTESLSFWRFIHVHARSEHLKEMSAVFGNPEIAKFRITHIDKLVEDPTSLNIAMGMSPANLLKTEVKKCLIESRQTIRNQVIKDATIYLYHEEDRLRSFLWSINPLFPRFLSEFKSGTFLGVADGLISLFQNSRTIRNSFKKKYHRELDDLIVRSEVSSLTHLGKLHLRRGSCKMWTCSATHADTLRYKSWGRTVIGTTVPHPLEMLGPQHRKETPCAPCNTSGFNYVSVHCPDGIHDVFSSRGPLPAYLGSKTSESTSILQPWERESKVPLIKRATRLRDAISWFVEPDSKLAMTILSNIHSLTGEEWTKRQHGFKRTGSALHRFSTSRMSHGGFASQSTAALTRLMATTDTMRDLGDQNFDFLFQATLLYAQITTTVARDGWITSCTDHYHIACKSCLRPIEEITLDSSMDYTPPDVSHVLKTWRNGEGSWGQEIKQIYPLEGNWKNLAPAEQSYQVGRCIGFLYGDLAYRKSTHAEDSSLFPLSIQGRIRGRGFLKGLLDGLMRASCCQVIHRRSLAHLKRPANAVYGGLIYLIDKLSVSPPFISLTRSGPIRDELETIPHKIPTSYPTSNRDMGVIVRNYFKYQCRLIEKGKYRSHYSQLWLFSDVLSIDFIGPFSISTTLLQILYKPFLSGKDKNELRELANLSSLLRSGEGWEDIHVKFFTKDILLCPEEIRHACKFGIAKDNNKDMSYPPWGRESRGTITTIPVYYTTTPYPKMLEMPPRIQNPLLSGIRLGQLPTGAHYKIRSILHGMGIHYRDFLSCGDGSGGMTAALLRENVHSRGIFNSLLELSGSVMRGASPEPPSALETLGGDKSRCVNGETCWEYPSDLCDPRTWDYFLRLKAGLGLQIDLIVMDMEVRDSSTSLKIETNVRNYVHRILDEQGVLIYKTYGTYICESEKNAVTILGPMFKTVDLVQTEFSSSQTSEVYMVCKGLKKLIDEPNPDWSSINESWKNLYAFQSSEQEFARAKKVSTYFTLTGIPSQFIPDPFVNIETMLQIFGVPTGVSHAAALKSSDRPADLLTISLFYMAIISYYNINHIRVGPIPPNPPSDGIAQNVGIAITGISFWLSLMEKDIPLYQQCLAVIQQSFPIRWEAVSVKGGYKQKWSTRGDGLPKDTRISDSLAPIGNWIRSLELVRNQVRLNPFNEILFNQLCRTVDNHLKWSNLRRNTGMIEWINRRISKEDRSILMLKSDLHEENSWRD